(19) United States
(12) Patent Application Publication
(10) Pub. No.: US 2025/0270328 A1
(43) Pub. Date: Aug. 28, 2025

(54) KLRB1 BINDING AGENTS AND METHODS OF USE THEREOF

(71) Applicant: The Brigham and Women's Hospital, Inc., Boston, MA (US)

(72) Inventor: Steven A. Greenberg, Boston, MA (US)

(21) Appl. No.: 18/866,615

(22) PCT Filed: May 18, 2023
(86) PCT No.: PCT/US2023/022735

§ 371 Date: Nov. 18, 2024

Related U.S. Application Data
(60) Provisional application No. 63/419,218, filed on Oct. 25, 2022.
(60) Provisional application No. 63/343,434, filed on May 18, 2022.

(51) Int. Cl. C07K 16/28; A61K 39/00

(52) U.S. Cl. CPC C07K 16/2851; A61K 2039/505; C07K 2317/24; C07K 2317/41; C07K 2317/732; C07K 2317/92

(57) ABSTRACT

KLRB1 binding agents (in particular anti-KLRB1 antibodies and antigen binding portion thereof) and compositions thereof, as well as therapeutic methods of using the agents, e.g., for depleting cells or inhibiting cells or activating cells (in particular, Th17, Th17.1, ex-Th17, Tc17, MAIT, INKT, peTh2, ILC2, ILC3, NK cells, and/or neoplastic T or NK cells in vivo), for the treatment of autoimmune disease, allergic diseases, transplant rejection, hematologic malignancies, and cancer.

### CLAIM OF PRIORITY

This application claims the benefit of U.S. Provisional Application Ser. No. 63/419,218, filed on Oct. 25, 2022 and U.S. Provisional Application Ser. No. 63/343,434, filed on May 18, 2022. The entire contents of the foregoing are incorporated herein by reference.

### STATEMENT REGARDING FEDERALLY SPONSORED RESEARCH

This invention was made with government support under HL119145 awarded by the National Institutes of Health. The government has certain rights in the invention.

### TECHNICAL FIELD

This disclosure generally relates to KLRB1 binding agents, in particular anti-KLRB1-antibodies as well as therapeutic methods of using the agents for autoimmune disease, allergic diseases, transplant rejection, hematologic malignancies, and cancer.

### BACKGROUND

The expression of killer cell lectin-like receptor subfamily B, member 1 (KLRB1; also known as CD161) defines a unique population of immune cells implicated in a variety of autoimmune and allergic diseases. For example, T-helper IL-17 secreting (Th17) cells express KLRB1 (Maggi et al. 2010). Th17 cells, and the cytokine IL-17 they produce, are implicated in psoriasis, psoriatic arthritis, ankylosing spondylitis, and inflammatory bowel disease (Yang et al. 2014), among other autoimmune diseases. Monoclonal antibody therapeutics targeting IL-17, or the upstream cytokine IL-23 that stimulates IL-17 producing cells, are FDA approved and marketed for use in psoriasis (e.g., secukinumab and ixekizumab) and ankylosing spondylitis (e.g., secukinumab). However, therapeutic developments based upon directly targeting these molecules remain limited and there remains a need for new therapeutics that reduce the activity of these cytokines for various indications including autoimmune disease.

### SUMMARY OF THE DISCLOSURE

The present invention provides a solution to the problem set forth above by providing antibodies and antigen-binding fragments thereof that bind to cells that express KLRB1 on their cell surface and target those bound cells for depletion. Those targeted cells directly produce or cause other cells to produce the cytokines described above. By depleting those cells, cytokine production is reduced providing a therapeutic benefit.

Described herein are KLRB1-binding antibodies (and antigen-binding fragments thereof) that have numerous uses, including therapeutic and diagnostic uses. For example, the antibodies and antigen-binding fragments thereof can be used for treating, and in some cases preventing, various diseases associated with KLRB1 expressing cells (i.e., by depleting the KLRB1 expressing cells), e.g., for treating, and in some cases preventing (i.e., reducing the risk of developing), autoimmune diseases including psoriasis, psoriatic arthritis, ankylosing spondylitis, palmoplantar pustulosis, hidradenitis suppurativa, and inflammatory bowel disease; allergic diseases including asthma and atopic dermatitis; transplant rejection; hematologic malignancies, and cancer. Antigen-binding fragments (also referred to herein as antibody fragments) include those described herein, e.g., Fab, Fab′, F(ab′)₂, Fv, and single chain antibodies (e.g., scFv).

Provided herein are antibodies or antigen binding portions thereof that specifically bind to killer cell lectin-like receptor subfamily B, member 1, (KLRB1; optionally SEQ ID NO: 1 or SEQ ID NO: 2), wherein the antibody or antigen binding portion thereof comprises or consists of at least one of (a) a heavy chain variable region (VH) comprising a VH complementarity determining region (CDR) 1 comprising a sequence that is at least 95% identical to a VH CDR1 amino acid sequence set forth in one of Table 1 to 53, preferably Table 1, 30, 31, or 32; a VH CDR2 comprising a sequence that is at least 95% identical to a VH CDR2 amino acid sequence set forth in one of Table 1 to 53, preferably Table 1, 30, 31, 32, or 45 to 53 and a VH CDR3 comprising a sequence that is at least 95% identical to a VH CDR3 amino acid sequence set forth in one of Table 1 to 53, preferably Table 1, 30, 31, 32, or 45 to 53; and (b) a light chain variable region (VL) comprising a VL CDR1 comprising a sequence that is at least 95% identical to a VL CDR1 amino acid sequence set forth in one of Table 1 to 53, preferably Table 1, 30, 31, 32, or 45 to 53, a VL CDR2 comprising a sequence that is at least 95% identical to a VL CDR2 amino acid sequence set forth in one of Table 1 to 53, preferably Table 1, 30, 31, 32, or 45 to 53, and a VL CDR3 comprising a sequence that is at least 95% identical to a VL CDR3 amino acid sequence set forth in one of Table 1 to 53, preferably Table 1, 30, 31, 32, or 45 to 53.

In some embodiments, the VH comprises or consists of an VH amino acid sequence set forth in Table 1, 30, 31, 32, or 45 to 53. In some embodiments, the VL comprises or consists of an VL amino acid sequence set forth in Table 1, 30, 31, 32, or 45 to 53. In some embodiments, the VH comprises or consists of the VH amino acid sequence set forth in Table 1, and the VL comprises or consists of the VH amino acid sequence set forth in Table 1. In some embodiments, the VH comprises or consists of the VH amino acid sequence set forth in Table 30, and the VL comprises or consists of the VH amino acid sequence set forth in Table 30. In some embodiments, the VH comprises or consists of the VH amino acid sequence set forth in Table 31, and the VL comprises or consists of the VH amino acid sequence set forth in Table 31. In some embodiments, the VH comprises or consists of the VH amino acid sequence set forth in Table 32, and the VL comprises or consists of the VH amino acid sequence set forth in Table 32. In some embodiments, the VH comprises or consists of the VH amino acid sequence set forth in Table 45, and the VL comprises or consists of the VH amino acid sequence set forth in Table 45. In some embodiments, the VH comprises or consists of the VH amino acid sequence set forth in Table 46, and the VL comprises or consists of the VH amino acid sequence set forth in Table 46. In some embodiments, the VH comprises or consists of the VH amino acid sequence set forth in Table 47, and the VL comprises or consists of the VH amino acid sequence set forth in Table 47. In some embodiments, the VH comprises or consists of the VH amino acid sequence set forth in Table 48, and the VL comprises or consists of the VH amino acid sequence set forth in Table 48. In some embodiments, the VH comprises or consists of the VH amino acid sequence set forth in Table 49, and the VL comprises or consists of the VH amino acid sequence set forth in Table 49. In some embodiments, the VH comprises or consists of the VH amino acid sequence set forth in Table 50, and the VL comprises or consists of the VH amino acid sequence set forth in Table 50. In some embodiments, the VH comprises or consists of the VH amino acid sequence set forth in Table 51, and the VL comprises or consists of the VH amino acid sequence set forth in Table 51. In some embodiments, the VH comprises or consists of the VH amino acid sequence set forth in Table 52, and the VL comprises or consists of the VH amino acid sequence set forth in Table 52. In some embodiments, the VH comprises or consists of the VH amino acid sequence set forth in Table 53, and the VL comprises or consists of the VH amino acid sequence set forth in Table 53. In some embodiments, the VH comprises or consists of an amino acid sequence having at least 95% sequence identity to the VH amino acid sequence set forth in Table 1, and the VL comprises or consists of an amino acid sequence having at least 95% sequence identity to the VL amino acid sequence set forth in Table 1. In some embodiments, the VH comprises or consists of an amino acid sequence having at least 95% sequence identity to the VH amino acid sequence set forth in one of Tables 30, 31, 32, or 45 to 53, and the VL comprises or consists of an amino acid sequence having at least 95% sequence identity to the amino acid sequence set forth in one of Tables 30, 31, 32, or 45 to 53, preferably wherein the VH and VL are from the same Table.

In some embodiments, the antibodies or antigen binding portions thereof comprise a constant region heavy chain and light chain, wherein the constant region heavy chain and/or light chain comprises or consists of an amino acid sequence set forth in one of Table 58.

Additionally, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 1 and a variable region light chain consisting of the LC amino acid sequence set forth in Table 1, and optionally a constant region, optionally wherein the antibody or antigen binding portion thereof comprises a sequence that is at least 95% identical to a sequence set forth in Table 57.

Also provided herein are antibodies or antigen binding portions thereof that specifically bind to human killer cell lectin-like receptor subfamily B, member 1, (KLRB1; SEQ ID NO:1), comprising or consisting of a variable region heavy chain consisting of the HC amino acid sequence set forth in Table 30 and a variable region light chain consisting of the LC amino acid sequence set forth in Table 30, and optionally a constant region, optionally wherein the antibody or antigen binding portion thereof comprises a sequence that is at least 95% identical to a sequence set forth in Table 57.

Additionally, provided herein are antibodies or antigen binding portions thereof that specifically bind to human killer cell lectin-like receptor subfamily B, member 1, (KLRB1; SEQ ID NO: 1), comprising or consisting of a variable region heavy chain consisting of the HC amino acid sequence set forth in Table 31, and a variable region light chain consisting of the LC amino acid sequence set forth in Table 31, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 32, and a variable region light chain consisting of the LC amino acid sequence set forth in Table 32, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 45, and a variable region light chain consisting of the LC amino acid sequence set forth in Table 45, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 46, and a variable region light chain consisting of the LC amino acid sequence set forth in Table 46, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 47, and a variable region light chain consisting of the LC amino acid sequence set forth in Table 47, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 48, and a variable region light chain consisting of the LC amino acid sequence set forth in Table 48, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 49, and a variable region light chain consisting of the LC amino acid sequence set forth in Table 49, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 50, and a variable region light chain consisting of the LC amino acid sequence set forth in Table 50, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 51, and a variable region light chain consisting of the LC amino acid sequence set forth in Table 51, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; SEQ ID NO: 1), comprising or consisting of a variable region heavy chain consisting of the HC amino acid sequence set forth in Table 52, and a variable region light chain consisting of the LC amino acid sequence set forth in Table 52, and optionally a constant region, optionally wherein the antibody or antigen binding portion thereof comprises a sequence that is at least 95% identical to a sequence set forth in Table 57.

Further, provided herein are antibodies or antigen binding portions thereof that specifically bind to human killer cell lectin-like receptor subfamily B, member 1, (KLRB1; e.g., SEQ ID NO:1 or 2), comprising or consisting of a variable region heavy chain consisting of the HC amino acid sequence set forth in Table 53, and a variable region light chain consisting of the LC amino acid sequence set forth in Table 53, and optionally a constant region, optionally wherein the antibody or antigen binding portion thereof comprises a sequence that is at least 95% identical to a sequence set forth in Table 57.

In some embodiments, the constant region comprises or consists of a sequence as set forth in Table 58.

In some embodiments, the antibodies comprise or consist of a heavy chain variable and/or light chain variable sequence that is at least 95% identical to a sequence set forth in Table 56.

In some embodiments, the antibodies comprise or consist of a complete heavy chain and/or light chain sequence that is at least 95% identical to a sequence set forth in Table 57.

Also provided herein are antibodies or antigen binding portions thereof that specifically bind to human KLRB1, comprising CDRs from different tables herein, or heavy/light chain pairs from different tables herein. Preferably, the antibodies or antigen-binding portions thereof bind to KLRB1 expressing cells and target them for depletion, e.g., via presence of an Fc domain for ADCC- or CDC-dependent depletion or conjugation to a cytotoxic agent.

In some embodiments, the antibodies or antigen binding portions thereof comprise a heavy chain constant region hinge region and Fc domain.

In some embodiments, the antibody or antigen binding portion thereof is an antibody that comprises a heavy chain constant region comprising an amino acid sequence having at least 80%, 90%, 95%, or 97% sequence identity to a heavy chain constant region amino acid sequence set forth in Table 58.

In some embodiments, the antibody or antigen binding portion thereof is a monoclonal antibody.

In some embodiments, the antibody or antigen binding portion thereof is a chimeric, humanized, or human antibody, and/or comprises one or more mutations (e.g., in a CDR) that remove Asn (N)-glycosylation sites or remove Cys, Asp, Met, Trp or Lys.

In some embodiments, the antibody or antigen binding portion thereof is an immunoglobulin G (IgG) subtype IgG1 antibody, an IgG2 antibody, or an IgG4 antibody.

In some embodiments, the antibody or antigen binding portion thereof is an antibody that comprises an Fc region, preferably human IgG1, that binds to Fc gamma receptors (FcγRs) and induces antibody dependent cell-mediated cytotoxicity (ADCC) to deplete cells expressing KLRB1, or that binds to C1q and induce complement dependent cytotoxicity (CDC).

In some embodiments, the antibody or antigen binding portion thereof is an antigen-binding fragments (also referred to herein as antibody fragments) including those described herein, e.g., Fab, Fab′, F(ab′)₂, Fv, and single chain antibodies (e.g., scFv).

In some embodiments, the antibody or antigen binding portion thereof is attached to a second molecule, e.g., a cytotoxic agent or moiety, or a detectable substance or molecule that allows the antibody to be used for diagnosis and/or detection. In some embodiments, the antibody or antigen binding portion thereof is conjugated to a cytotoxic agent or moiety to deplete or promote the depletion of cells expressing KLRB1.

In some embodiments, the antibody or antigen binding portion thereof comprises an Fc region that is afucosylated.

Also provided herein are polynucleotides comprising a nucleic acid sequence encoding an antibody or antigen binding portion thereof as described herein. In some embodiments, the nucleic acid sequence is operably linked to a promoter.

Additionally provided are vectors comprising the polynucleotides described herein, as well as host cells comprising the polynucleotides or the vectors, and optionally expressing an antibody or antigen binding portion thereof as described herein. Also provided herein are methods for making the antibodies or antigen binding portions thereof as described herein. The methods can include culturing the host cells under conditions sufficient to express the antibody or antigen binding portion thereof and isolating the antibody or antigen binding portion thereof. In some embodiments, the methods include formulating the antibody as a pharmaceutical composition.

Further, provided herein are pharmaceutical compositions that comprise an antibody or antigen binding portion thereof of as described herein (e.g., comprises or consists of the antibody or antigen binding portion thereof as an active agent), and a pharmaceutically acceptable carrier or diluent.

In some embodiments, the antibody or antigen binding portion thereof is not clone B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7).

Also provided herein are methods for treating one or more of an autoimmune disease, an allergic disease, a transplant rejection, and a hematologic malignancy in a subject in need thereof, the method comprising administering to the subject an antibody that binds KLRB1, preferably an antibody or antigen binding portion thereof, polynucleotide, vector, pharmaceutical composition, or host cell that expresses the antibody or antigen binding portion thereof, as described herein. Also provided are an antibody that binds KLRB1, preferably an antibody or antigen binding portion thereof or pharmaceutical composition as described herein, for use in a method of treating one or more of an autoimmune disease, an allergic disease, a transplant rejection, and a hematologic malignancy in a subject in need thereof. Also provided are polynucleotide, vector, or host cell that expresses the antibody or antigen binding portion thereof, e.g., for use in cell or gene therapy. In some embodiments, the autoimmune disease is autoimmune hepatitis, rheumatoid arthritis, Sjogren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, or hidradenitis suppurativa. In some embodiments, the allergic disease is asthma, allergic eosinophilic asthma, allergy, atopic dermatitis, nasal polyposis, eosinophilic gastrointestinal disorder, or hypereosinophilic syndrome. In some embodiments, the transplant rejection can be a rejection of a kidney, lung, heart, liver, limb, skin, or multi-organ transplant. In some embodiments, the hematological malignancy is a leukemia, e.g., T cell leukemia, NK cell leukemia, T cell prolymphocytic leukemia (T-PLL), or large granular lymphocytic leukemia (LGLL). In some embodiments, the hematological malignancy is a lymphoma, e.g., hepatosplenic T cell lymphoma, NK/T cell lymphoma, mycosis fungoides, Sezary syndrome, peripheral T cell lymphoma, angioimmunoblastic T cell lymphoma (AITL), or peripheral T cell lymphoma not otherwise specified (PTCL-NOS). In some embodiments, the hematological malignancy expresses KLRB1.

In some embodiments, the antibody or antigen binding portion thereof binds to and depletes Th17, Th17.1, ex-Th17, Tc17, mucosal-associated invariant T cells (MAIT), invariant NK-T cells (iNKT), innate lymphoid cells types 2 and 3 (ILC2, and ILC3), pathogenic effector Th2 (peTh2) cells, and/or NK cells expressing KLRB1. In some embodiments, the antibody or antigen binding portion thereof is conjugated to a cytotoxic agent or moiety.

In some embodiments, the antibody or antigen binding portion thereof used in the methods of treatment described herein is, or is not, clone B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7).

In some embodiments of each of the aforementioned aspects and embodiments, as well as other aspects and embodiments described herein, the subject is human.

Without wishing to be being bound by theory, the inventors believe that the antibodies disclosed herein will demonstrate superiority to previously described antibodies, e.g., commercially available antibodies, in one or more of the following activities:
increased binding potency (lower binding EC₅₀ to cell expressed KLRB1 or to soluble KLRB1 extracellular domain);
increased ADCC mediated depletion potency (lower EC₅₀ for depletion of CHO-KLRB1+ cells or lower EC₅₀ for reporter cell line ADCC assay);
increased binding affinity kinetics (lower KD or lower K_{off});
increased blocking potency (lower IC₅₀ for inhibition of CLEC2D binding to cell expressed KLRB1); and/or
increased augmentation potency (lower EC₅₀ for augmenting (increasing) CLEC2D binding to cell expressed KLRB1).

In some embodiments, the antibodies disclosed herein have a higher production yield, lower immunogenicity (due to the presence of humanized variable regions and/or human Fc sequences), and/or improved biophysical parameters (e.g., higher melting temperature, greater freeze-thaw stability, lower isomerization, reduced or absence of deamidation, and/or less susceptibility to oxidation) when compared to previously described antibodies, e.g., commercially available antibodies.

Where aspects or embodiments of the disclosure are described in terms of a Markush group or other grouping of alternatives, the present disclosure encompasses not only the entire group listed as a whole, but also each member of the group individually and all possible subgroups of the main group, and also the main group absent one or more of the group members. The present disclosure also envisages the explicit exclusion of one or more of any of the group members in the claimed disclosure.

These and other advantages of the present technology will be apparent when reference is made to the accompanying drawings and the following description.

### BRIEF DESCRIPTION OF THE DRAWINGS

FIG. 1 shows KLRB1 expression marks Th17, Th17.1, ex-Th17, Tc17, iNKTs, IL2, ILC3, peTh2, and a subset of NK cells.

FIG. 2 shows body-wide KLRB1 expression profiling data indicating that KLRB1 has no significant expression on any cell type other than immune cells

FIG. 3 shows KLRB1 is overexpressed in rheumatoid arthritis synovium compared to osteoarthritis and normal synovium.

FIG. 4 shows KLRB1 is overexpressed in rheumatoid arthritis synovium compared to osteoarthritis and crystal-induced arthritis synovium.

FIG. 5 shows KLRB1 is overexpressed in advanced and moderate Sjögren's syndrome salivary gland compared to normal.

FIG. 6 shows KLRB1 is overexpressed in Sjögren's syndrome parotid gland compared to normal.

FIG. 7 shows KLRB1 is overexpressed in muscle tissue from patients with inclusion body myositis.

FIG. 8 shows KLRB1 is overexpressed in skin tissue from patients with discoid lupus.

FIG. 9 shows KLRB1 is overexpressed in skin tissue from patients with psoriasis.

FIG. 10 shows KLRB1 is overexpressed in lung tissue from patients with idiopathic pulmonary fibrosis.

FIG. 11 shows KLRB1 is overexpressed in pancreas tissue from patients with diabetes.

FIG. 12 shows KLRB1 is overexpressed in scalp tissue from patients with alopecia universalis.

FIG. 13 shows KLRB1 is overexpressed in liver tissue from patients with primary biliary cholangitis.

FIG. 14 shows KLRB1 is overexpressed in brain tissue from patients with multiple sclerosis.

FIG. 15 shows KLRB1 is overexpressed in colon tissue from patients with lymphocytic colitis.

FIG. 16 shows KLRB1 is overexpressed in kidney tissue from patients with renal transplants.

FIG. 17 shows KLRB1 is overexpressed in bronchoalveolar lavage fluid from patients with lung transplants.

FIG. 18 shows KLRB1 is overexpressed in skin tissue from patients with atopic dermatitis.

FIG. 19 shows KLRB1 is overexpressed in skin tissue from patients with palmoplantar pustulosis.

FIG. 20 shows KLRB1 is overexpressed in skin tissue from patients with hidradenitis suppurativa.

FIG. 21 shows KLRB1 is overexpressed in airway brushings from patients with asthma.

FIGS. 22A-D shows KLRB1 is expressed in various T and NK cell lymphomas and leukemias. FIG. 22A shows increased expression of KLRB1 in tumor cells from 4/4 patients with hepatosplenic T cell lymphoma (HSTCL), 7/19 patients with NK/T cell lymphoma (NKTCL), and 2/2 patients with mycosis fungoides. FIG. 22B shows expression of KLRB1 in various forms of peripheral T cell lymphoma (PTCL) including angioimmunoblastic T cell lymphoma (AITL), anaplastic large cell lymphoma (ALCL; both ALK-positive and ALK-negative), NK/T cell lymphoma (NKTCL), peripheral T cell lymphoma not otherwise specified (PTCL-NOS), and T cell prolymphocytic leukemia (T-PLL). FIG. 22C shows KLRB1 increased expression in spleen tumor cells compared to healthy spleen and from tumor cells compared to healthy CD4 T cells in 4 patients each with HSTCL. FIG. 22D shows increased expression of KLRB1 an HSTCL cell line (DERL-2) and from aggressive NK cell leukemia (ANKL) cell lines (KHYG-1 and NKL).

FIG. 23 shows binding of murine antibody 10A3D6 against human KLRB1 by ELISA. Human KLRB1 was coated on ELISA plates and incubated with 10A3D6. Secondary anti-mouse IgG was used to detect 10A3D6 bound to KLRB1 on the ELISA plate.

FIGS. 24A-B shows binding of humanized antibodies 10A3D6hum2.2 and 10A3D6hum2.2-afuc (afucosylated 10A3D6hum2.2) to CHO-K1 cells expressing human KLRB1 (CHO-hum-KLRB1) (FIG. 24A) and to CHO-K1 cells expressing cynomolgus KLRB1 (CHO-cyno-KLRB1) (FIG. 24B). Controls used were human IgG and PBS (negative controls). Binding was detected by FACS. Secondary anti-human IgG was used to detect antibodies bound to CHO-hum-KLRB1 or CHO-cyno-KLRB1.

FIGS. 25A-B shows an antibody dependent cell-mediated cytotoxicity (ADCC) assay using luminescence with increasing concentrations of Rituxan (positive control), test antibody, and human IgG1 antibody (negative control) using target cells+a reporter cell line GS-J2 that expresses CD16A (GS-J2/CD16A; Genscript, Inc.) FIG. 25A shows cell luminescence of 10A3D6-hIgGlk (a mouse/human chimeric KLRB1 binding antibody with 10A3D6 variable regions and human IgG1-kappa constant regions) and FIG. 25B 10A3D6-hIgGlk compared to a negative control antibody 52G9B12B5-hIgGlkEN (a mouse/human chimeric KLRB1-binding antibody with 52G9B12B5 variable regions and human IgG1-kappa mutant L234A/L235A/G237A mutation).

FIGS. 26A-B show an antibody dependent cell-mediated cytotoxicity (ADCC) assay measuring target cell lysis of CHO-K1 target cells expressing human KLRB1 (CHO-hum-KLRB1) incubated with human peripheral blood mononuclear cells (PBMCs) and the indicated antibodies 10A3D6-hIgGlk (a mouse/human chimeric antibody with 10A3D6 variable regions and human IgG1-kappa constant regions) and human IgG1 (negative control) in an ADCC cell lysis assay. The assay uses LDH release and detection of formazan salt (Genscript, SC1544). FIG. 26A shows % target cell lysis at various effector to target (E:T) ratios as indicated for each test antibody and control antibody and FIG. 26B shows % target cell lysis for each test antibody at the various concentrations indicated.

FIG. 27 shows an antibody dependent cell-mediated cytotoxicity (ADCC) assay measuring target cell death of CHO-K1 target cells expressing human KLRB1 (CHO-hum-KLRB1) incubated with human natural killer (NK) cells and the indicated antibodies 10A3D6hum2.2, 10A3D6hum2.2-afucosylated, and a negative control antibody 52G9B12B5-hIgG1kEN (a mouse/human chimeric KLRB1 binding antibody with 52G9B12B5 variable regions and human IgG1-kappa mutant L234A/L235A/G237A mutation). The assay uses flow cytometry to detect dead CHO-hum-KLRB1 cells.

FIGS. 28A-G show binding in terms of mean fluorescence intensity of the indicated antibodies (10A3D6hum1.2; 10A3D6hum1.5; and 10A3D6hum2.2; and human IgG1 control) to the various immune cells in whole blood taken from 3 different donors, i.e., CD3+ cells (FIG. 28A), CD4+ T cells (FIG. 28B), CD8+ T cells (FIG. 28C), CD3-CD56+ cells (FIG. 28D), CD14++ monocytes (FIG. 28E), CD14+neutrophils (FIG. 28F), and CD19+ cells (FIG. 28G).

FIGS. 29A-B show competition data between the 103AD6hum1.2 antibody and commercially available antibodies against cells expressing KLRB1. CHO-K1 cells expressing human KLRB1 (CHO-hum-KLRB1) (FIG. 29A) and CHO-K1 cells expressing cynomolgus KLRB1 (CHO-cyno-KLRB1) (FIG. 29B) were incubated with fluorescently labelled antibody 10A3D6hum1.2 and then with increasing concentrations of commercial antibodies HP-3G10, DX12, 191B8, and negative control mouse IgG and human IgG. The graphs in FIGS. 29A-B show mean fluorescent intensity (MFI) (1000s) over the indicated concentration range (nM).

FIGS. 30A-C show differential scanning calorimetry (DSC) graphs indicating Cp (kcal/mol/K) over the range of temperatures indicated, for 10A3D6hum1.2 (FIG. 30A), 10A3D6hum1.5 (FIG. 30B), and 10A3D6hum2.2 (FIG. 30C).

FIG. 31 shows depletion of KLRB1+ T cells in cynomolgus monkey administered 10A3D6hum2.2-afuc (afucosylated 10A3D6hum2.2) at 3 mg/kg and 10 mg/kg.

FIGS. 32A-C show depletion of KLRB1+ T cells in cynomolgus monkey by 10A3D6hum2.2-afuc (afucosylated 10A3D6hum2.2) at 0.1 mg/kg, 0.3 mg/kg, 1 mg/kg, and 3 mg/kg. These studies showed near complete depletion of the KLRB1+CD8+ T cell population at 8 hours post-dose, Day 1, 3, 7, and 35 (FIG. 32A and FIG. 32B). Gene expression of KLRB1 transcript by quantitative PCR (qPCR) similarly showed depletion of KLRB1 transcript (FIG. 32C).

FIG. 33 shows pharmacokinetic parameters of 10A3D6hum2.2-afuc in cynomolgus monkey.

FIG. 34 shows reduction in Th17 pathway gene expression by RNAseq (heatmap representation).

FIG. 35 shows reduction in Th17 pathway gene expression by RNAseq (graphical representation).

FIG. 36 shows reduction in peTh2 pathway gene expression by RNAseq (heatmap representation).

FIG. 37 shows reduction in peTh2 pathway gene expression by RNAseq (graphical representation).

FIG. 38 shows reduction in Th1 and CD8 effector pathway gene expression by RNAseq (heatmap representation).

FIG. 39 shows reduction in Th1 and CD8 effector pathway gene expression by RNAseq (graphical representation).

### DETAILED DESCRIPTION

KLRB1 expression marks a unique set of immune system cells implicated in a variety of autoimmune diseases. These include Th17, Th17.1, ex-Th17, Tc17, MAIT, iNKT, ILC2, ILC3, peTh2, and/or NK cells.

KLRB1 is also expressed by neoplastic cells present in a number of T and NK cell malignancies. These include various peripheral T cell and NK cell lymphomas and leukemias.

The KLRB1-binding antibodies described herein can be used to preferentially target Th17, Th17.1, ex-Th17, Tc17, MAIT, INKT, ILC2, ILC3, peTh2, NK cells, and/or neoplastic T or NK cells for depletion. The population of KLRB1 expressing immune cells more abundantly expresses IL-17 than the population of total CD4 or CD8 T cells, and are more specific to Th17 and Tc17 T cells than CD4 or CD8, respectively. Thus described herein are methods for treating a subject by administering to a subject in need thereof an effective amount of a killer cell lectin-like receptor B1 (KLRB1) binding agent with cell depleting activity, thereby depleting Th17, Th17.1, ex-Th17, Tc17, MAIT, INKT, ILC2, ILC3, peTh2, NK cells, and/or neoplastic T or NK cells in vivo. These methods can be used to treat an autoimmune disease, allergic disease, transplant rejection, or hematologic malignancies, e.g., as described herein.

Additionally, in some embodiments, because modulating KLRB1 has inhibitory or stimulatory effects on T and NK cells, thus described herein are methods for treating a subject by administering to a subject in need thereof an effective amount of a killer cell lectin-like receptor B1 (KLRB1) binding agent with KLRB1 receptor blocking activity (in which blocking of binding of LLT1 to KLRB1 is achieved), e.g., an antibody as described herein with effector null Fc mutations (e.g., as described in Liu et al., Antibodies (Basel). 2020 December; 9 (4): 64), thereby activating or inhibiting Th17, Th17.1, ex-Th17, Tc17, MAIT, INKT, ILC2, ILC3, peTh2, and/or in vivo. These methods can be used to activate T and/or NK cells to enhance their activity against tumor cells to be used to treat cancer.

In some embodiments, the antibodies have antibody dependent cellular cytotoxicity (ADCC) effector activity or complement dependent cytotoxicity (CDC) effector activity. Administering to a subject in need thereof an effective amount of an anti-KLRB1 antibody that has ADCC or CDC effector function, or that is linked to a cytotoxic agent, can be used to eliminate or reduce the number of Th17, Th17.1, ex-Th17, Tc17, MAIT, INKT, ILC2, ILC3, peTh2, NK cells, and/or neoplastic T or NK cells. In some embodiments, the disclosure provides a killer cell lectin-like receptor B1 (KLRB1) binding agent as described herein that has ADCC or CDC activity or is conjugated to a cytotoxic agent. In various aspects, the disclosure provides an mRNA or cDNA encoding the binding agent. In various aspects, the disclosure provides a pharmaceutical composition comprising an effective amount of the binding agent.

Various features of the disclosure, including KLRB1 and its ligands, anti-KLRB1 antibodies or antigen-binding portions thereof, pharmaceutical compositions, treatment and administration, and illustrative examples are discussed, in turn, below. The following sections contain definitions of terms used in this disclosure. Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

### Killer Cell Lectin-Like Receptor B1 (KLRB1)

Killer cell lectin-like receptor B1 (KLRB1), also known as CD161, is a type II transmembrane protein. It is a receptor modulating the activity of T and NK cells and can act as either a stimulatory (Fergusson et al. 2014) or inhibitory (Aldemir et al. 2005, Mathewson et al. 2021) receptor for different types of T and NK cells. It is present on the surface of a variety of immune cells. The ligand for KLRB1 is LLT1 (also called CLEC2D) (Aldemir et al. 2005).

KLRB1 expression among T cells is limited to those with an ability to respond to IL-12 and IL-18 (Fergusson et al. 2014). Among CD4+ T helper cells, KLRB1 expression uniquely marks Th17, Th17.1, and ex-Th17 cells (cells that no longer produce IL-17 but produce IFN-γ). KLRB1 expression distinguishes Th1 IFNG producing cells (which are KLRB1-negative) from ex-Th17 IFNG producing cells (which are KLRB1+) (Basdeo et al. 2017). Among CD8+ T cytotoxic cells, KLRB1 expression uniquely marks Tc17 cells. Thus, KLRB1 marks T cells with capacity for IL-17 production or, in the case of ex-Th17 cells, interferon-gamma (IFNG) production. This cytokine production is undesired in the case of autoimmune disease.

Described herein are antibodies and antigen-binding fragments thereof that bind to KLRB1, preferably human KLRB1. An exemplary sequence of human KLRB1 is provided as SEQ ID NO:1:

MDQQAIYAELNLPTDSGPESSSPSSLPRDVCQGSPWHQFALKLSCAGIIL
LVLVVTGLSVSVTSLIQKSSIEKCSVDIQQSRNKTTERPGLLNCPIYWQQ
LREKCLLFSHTVNPWNNSLADCSTKESSLLLIRDKDELIHTQNLIRDKAI
LFWIGLNFSLSEKNWKWINGSFLNSNDLEIRGDAKENSCISISQTSVYSE
YCSTEIRWICQKELTPVRNKVYPDS.

In some embodiments, the KLRB1 is Cynomolgus KLRB1; an exemplary sequence is provided as SEQ ID NO:2:

MDQQMMYAELTLPKDSGPESSSPSSLPRDVCQGSPWHQFALKLSCAGIIL
LVLVVTGLSLSVASLLQKPSIGKCSVDIQQNRTKTTERPDLLNCPIYWQQ
VQEKCLLFSHTVNPWNNSLADCSTKESSLLLIQDKDELTRTQNLIHDKAI
SFWIGLNFSLSEKNWKWINGSFLSSNDLKITGDAKENSCVYISQTSVYSE
YCSTEMKWICQKELTLVRNKVSPDSWL.

Preferably, the antibodies bind to cells that express KLRB1 on their surface (e.g., Th17, Th17.1, ex-Th17, Tc17, MAIT, iNKT, ILC2, ILC3, peTh2, NK cells, and/or neoplastic T or NK cells) and target such cells for depletion, e.g., via ADCC or CDC (e.g., for full length antibodies comprising an Fc region) or a cytotoxic agent or moiety (e.g., for antigen-binding fragments lacking an ADCC- or CDC-competent Fc region).

### KLRB1-Binding Antibodies

Described herein are antibodies and antigen binding fragments thereof that bind to KLRB1. The term “antibody” refers to an immunoglobulin molecule or immunologically active portion thereof, i.e., an antigen-binding portion.

The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody. The term “monoclonal antibody” encompasses intact and full-length monoclonal antibodies as well as antibody fragments (e.g., Fab, Fab′, F(ab′)₂, Fv, and single chain antibodies (e.g., scFv)), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region residues of the human heavy chain or light chain variable regions are replaced by corresponding residues from nonhuman antibody. Furthermore, humanized antibodies can comprise residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

“Antibody fragments” or “antigen-binding fragments” can include a portion of an intact antibody, preferably the antigen binding or variable region of the intact antibody. Examples of antibody fragments include Fab, Fab′, F(ab′)₂, and Fv fragments; diabodies; linear antibodies; single-chain antibody molecules (e.g., scFv); and multispecific antibodies formed from antibody fragments. In some embodiments, the antigen-binding fragments is attached to a second molecule, e.g., a cytotoxic agent or moiety, e.g., that promotes depletion of KLRB1+ cells to which the antigen-binding fragments bind, or a detectable substance or molecule that allows the antibody to be used for diagnosis and/or detection.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (CH1) of the heavy chain. Fab fragments differ from Fab′ fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgB1, IgG2, IgG3, IgG4, IgA, and IgA2. “Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding.

In various embodiments, the antibody or antigen binding fragment thereof comprises a human or humanized antibody. Humanized forms of non-human (e.g., murine) antibodies are chimeric immunoglobulins, immunoglobulin chains or fragments thereof (such as Fv, scFv, Fab, Fab′, F(ab′)2 or other antigen-binding subsequences of antibodies) that contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

The KLRB1 antibodies described herein can be affinity matured, for example using selection and/or mutagenesis methods known in the art. In general, an “affinity matured” antibody is one with one or more alterations in one or more hyper variable regions thereof which result in an improvement in the affinity of the antibody for antigen, compared to a parent antibody which does not possess those alteration(s). In one embodiment, an affinity matured antibody has nanomolar or even picomolar affinities for the target antigen. Preferred affinity matured antibodies have an affinity that is five times, more preferably 10 times, even more preferably 20 or 30 times greater than the starting antibody (generally murine, humanized or human) from which the matured antibody is prepared.

An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. The term “specifically binds” as used herein refers to a KLRB1 agent (e.g., an anti-KLRB1 antibody) that interacts more frequently, more rapidly, with greater duration, with greater affinity, or with some combination of the above to a particular antigen, epitope, protein, or target molecule than with alternative substances. A binding agent (e.g. antibody) that specifically binds an antigen can be identified, for example, by immunoassays, ELISAs, Surface Plasmon Resonance (SPR) assays (e.g., Biacore), or other techniques known to those of skill in the art. As such, described herein are functional equivalents to the specific anti-KLRB1 antibodies described. In some cases, the KLRB1 antibody may be cross reactive with various similar KLRB1 proteins (e.g., with highest affinity for one, such as human KLRB1, and lower affinity for others, such as mouse KLRB1). A binding agent that specifically binds an antigen binds the target antigen with a higher affinity than its affinity for a different antigen. The different antigen can be a related antigen. In some embodiments, a binding agent that specifically binds an antigen binds the target antigen with an affinity that is at least 20 times greater than its affinity for a different antigen, e.g., at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology, e.g., in a Biacore system or other system known to those of skill in the art.

The terms “identical” or “percent identity” in the context of two or more polypeptides (e.g., two anti-KLRB1 antibodies), refer to two or more sequences or subsequences that are the same or have a specified percentage of amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof.

The percent identity between two sequences is a function of the number of identical positions shared by the sequences (i.e., % homology= # of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm. Such homology is well-represented in the art via local alignment tools and/or algorithms, and may include pairwise alignment, multiple sequence alignment methods, structural alignment methods, and/or phylogenetic analysis methods. Where sequences differ in conservative substitutions, the percent sequence identity may be, but not necessarily is, adjusted upwards to correct for the conservative nature of the substitution. Means for making this adjustment are well known to those of skill in the art. Typically, but not necessarily, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1.

In some embodiments, two polypeptides (e.g., antibodies or antibody domains (e.g., VL, CL, VH, CH1, CH2, CH3 domains) thereof) of the disclosure are substantially identical, meaning they have at least 70%, at least 75%, at least 80%, at least 85%, at least 90%, and in some embodiments at least 95%, 96%, 97%, 98%, 99% amino acid residue identity, when compared and aligned for maximum correspondence, as measured using a sequence comparison algorithm or by visual inspection. In some embodiments, percent identity exists over a region of the sequences that is at least about 10, at least about 20, at least about 20-40, at least about 40-60 amino acid residues, at least about 60-80 nucleotides or amino acid residues in length or any integral value there between. In some embodiments, percent identity exists over a longer region than 60-80 amino acid residues, such as at least about 80-100 amino acid residues, and in some embodiments the sequences are substantially identical over the full length of the sequences being compared, for example, an amino acid sequence.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

The terms “conservative sequence modifications” or “conservative substitutions” as used herein may refer to amino acid modifications to a target epitope or antibodies and antigen-binding portions thereof of the disclosure that does not significantly affect or alter the binding characteristics of the anti-KLRB1 antibodies. “Conservative substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Methods of identifying amino acid conservative substitutions that do not eliminate binding are well-known in the art.

In various embodiments, the antibody is a blocking or antagonist binding agent. “Blocking” or “antagonist” means the agent (e.g., antibody or binding fragment thereof) is one that inhibits or reduces biological activity of the antigen it binds. Certain blocking agents or antagonist agents substantially or completely inhibit the biological activity of the antigen. For example, a KLRB1 binding agent as described herein with effector null Fc mutations can block KLRB1 signaling (e.g., thereby disrupting KLRB1 signaling and modulating Th17, Th17.1, ex-Th17, Tc17, MAIT, iNKT, ILC2, ILC3, peTh2, NK cells, and/or neoplastic T or NK cells cells).

In some embodiments, the KLRB1 binding agent is an antibody that comprises: a. a full length antibody that binds KLRB1 and comprises an Fc domain that can bind Fcgamma receptors with effector function to trigger antibody-dependent cell-mediated cytotoxicity (ADCC); b. an antibody that binds KLRB1 and comprises an Fc domain that can bind complement protein 1q (C1q) with effector function to trigger complement dependent cytotoxicity (CDC); c. an antibody conjugate that binds KLRB1 and comprises a cytotoxic agent, e.g., antibody-drug conjugate (ADC); or d. a multispecific antibody (e.g., a bispecific or trispecific antibody), wherein the antibody binds to KLRB1 and another antigen.

In some embodiments, the KLRB1 antibodies described herein bind to the extracellular domain of human KLRB1; in some embodiments, the antibody cross-reacts with (binds to both of) the extracellular domains of human and cynomolgus KLRB1. In some embodiments, the antibody binds to an epitope of the extracellular domain of KLRB1, wherein the epitope is at least 90% identical in human and cynomolgus. Preferably, the antibodies or antigen-binding fragments thereof comprise or are conjugated to an Fc region, a cytotoxic agents, or a second antigen-binding domain that cause depletion of KLRB1-expressing cells.

In some embodiments, the antibody binds to KLRB1 and is not a mouse antibody.

In some embodiments, the antibody described herein is not B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7).

In some embodiments, a KLRB1-binding agent is an antibody, e.g., a full length antibody comprising an Fc domain including at least one heavy chain. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the antibody is an antibody fragment comprising an antigen-binding site. In some embodiments, the antibody is a scFv. In some embodiments, the antibody is a disulfide-linked scFv. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is isolated. In some embodiments, the antibody is substantially pure. In some embodiments, a KLRB1-binding agent is a polyclonal antibody. Polyclonal antibodies can be prepared by any method known to those of skill in the art. In some embodiments, polyclonal antibodies are produced by immunizing an animal (e.g., a rabbit, rat, mouse, goat, donkey) with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KLH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion. After a period of time, polyclonal antibodies are recovered from the immunized animal (e.g., from blood or ascites). In some embodiments, the polyclonal antibodies are purified from serum or ascites according to standard methods in the art including, but not limited to, affinity chromatography, ion-exchange chromatography, gel electrophoresis, and/or dialysis.

In some embodiments, a KLRB1-binding agent is a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are substituted for constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and affinity of a monoclonal antibody.

In some embodiments, a KLRB1-binding agent is a humanized antibody. Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some embodiments, a KLRB1-binding agent is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, a KLRB1-binding agent is a scFv antibody. ScFvs are molecules that comprise a variable heavy chain region and a variable light chain region linked to form a single polypeptide. ScFvs can be produced using recombinant technologies known in the art. In some embodiments, a scFv comprises a polypeptide linker between the heavy chain variable region and the light chain variable region. In some embodiments, the scFv comprises an orientation of (from N- to C-terminus) (i) heavy chain variable region, (ii) linker, and (iii) light chain variable region. In some embodiments, the scFv comprises an orientation (from N- to C-terminus) of (i) light chain variable region, (ii) linker, and (iii) heavy chain variable region. In some embodiments, the scFv is a disulfide-linked scFv (dsscFv), which is a scFv comprising an engineered disulfide bond between the light chain variable region and heavy chain variable region of the scFv. In some embodiments, the scFv (e.g., dsscFv) is attached (either directly or indirectly) to a half-life extending moiety such as, e.g., an Fc molecule, a CH3 domain of an immunoglobulin (e.g., CH3 of IgG1), polyethylene glycol (PEG) or a PEG mimetic, XTEN, serum albumin (e.g., human serum albumin), polysicalic acid, N-(2-hydroxypropyl) methacrylamide, or dextran, or is modified by, e.g., hyperglycosylation, to extend the half-life of the scFv (e.g., dsscFv). In some embodiments, the scFv is attached to a second molecule, e.g., a cytotoxic agent or moiety, or a detectable substance or molecule that allows the antibody to be used for diagnosis and/or detection.

A variety of suitable linkers are known to those of skill in the art and are not limited by any specific sequences disclosed herein. In some embodiments, the polypeptide linker is comprised of naturally, or non-naturally, occurring amino acids. In some embodiments, the linker comprises amino acids that allow for flexibility. In some embodiments, the linker comprises amino acids that allow for suitable solubility. In some embodiments, the linker comprises glycine amino acids. In some embodiments, the linker comprises glycine and serine amino acids. In certain embodiments, the linker comprises one or more sets of glycine/serine repeats. In some embodiments, the polypeptide linker is selected from the group consisting of: (GGGGS) n wherein n=1-4 (SEQ ID NO:85), GGGGS (SEQ ID NO:86), GGGGSGGGGS (SEQ ID NO:87), GGGGSGGGGSGGGGS (SEQ ID NO:88), GGGGSGGGGSGGGGSGGGGS (SEQ ID NO:89), and (GGGGA) n wherein n=1-4 (SEQ ID NO: 90). In some embodiments, the linker comprises GGGGSGGGGSGGGGS (SEQ ID NO: 91).

In some embodiments, a KLRB1-binding agent is a Fv. A Fv comprises a heavy chain variable region and a light chain variable region. In some embodiments, the Fv is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the Fv. In some embodiments, the Fv is attached to a second molecule, e.g., a cytotoxic agent or moiety, or a detectable substance or molecule that allows the antibody to be used for diagnosis and/or detection.

In some embodiments, a KLRB1-binding agent is a Fab. A Fab is one of the molecules that result from digestion of an immunoglobulin antibody with papain. Fabs are monovalent molecules that comprise a light chain, a heavy chain variable region, a CHI region, and, optionally, a heavy chain constant region hinge region or a portion thereof. Fabs can be produced using recombinant technologies known in the art. In some embodiments, a Fab comprises a polypeptide linker between the light chain constant region and the heavy chain variable region. In some embodiments, a Fab comprises a polypeptide linker between the heavy chain constant region and the light chain variable region. A variety of suitable linkers are known to those of skill in the art and are not limited by any specific sequences disclosed herein. In certain embodiments, the linker is a linker described herein. In some embodiments, the Fab is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysialic acid, N-(2-hydroxypropyl) methacrylamide, or dextran, or is modified, e.g., by hyperglycosylation, to extend the half-life of the Fab. In some embodiments, the Fab is attached to a second molecule, e.g., a cytotoxic agent or moiety, or a detectable substance or molecule that allows the antibody to be used for diagnosis and/or detection.

In some embodiments, a Fab comprises a disulfide bond formed between the heavy chain variable region and the light chain variable region. In some embodiments, a Fab comprises a disulfide bond that increases stability of the Fab molecule. In some embodiments, a Fab comprises a disulfide bond that increases thermostability of the Fab molecule.

In some embodiments, a KLRB1-binding agent is a F(ab′)₂. A F(ab′)₂ is one of the molecules that results from digestion of an immunoglobulin antibody with pepsin. A F(ab′)₂ is a divalent molecule that comprises a first light chain in association with a first polypeptide comprising a first heavy chain variable region, a first CH1, and a first hinge region, and a second light chain in association with a second polypeptide comprising a second heavy chain variable region, a second CH1, and a second hinge region, wherein the first hinge region is linked to the second hinge region via at least one disulfide bond. F(ab′)₂s can be produced using recombinant technologies known in the art. In some embodiments, the F(ab′)₂ is attached (either directly or indirectly) to a half-life extending moiety such as, e.g., a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the F(ab′)₂. In some embodiments, the F(ab′)₂ is attached to a second molecule, e.g., a cytotoxic agent or moiety, or a detectable substance or molecule that allows the antibody to be used for diagnosis and/or detection.

In some embodiments, a F(ab′)₂ comprises a disulfide bond formed between the heavy chain variable region and the light chain variable region. In some embodiments, a F(ab′)₂ comprises a disulfide bond that increases stability of the F(ab′)₂ molecule. In some embodiments, a F(ab′)₂ comprises a disulfide bond that increases thermostability of the F(ab′)₂ molecule.

In some embodiments, a KLRB1-binding agent is a F(ab′). A F(ab′) is a molecule that results from treatment of a F(ab′)₂ with beta-mercaptoethanol. A F(ab′) is a monovalent molecule that comprises a light chain in association with a polypeptide comprising a heavy chain variable region, a CH1, and a hinge region. In some embodiments, the F(ab′) is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the F(ab′). In some embodiments, the F(ab′) is attached to a second molecule, e.g., a cytotoxic agent or moiety, or a detectable substance or molecule that allows the antibody to be used for diagnosis and/or detection.

In some embodiments, a F(ab′) comprises a disulfide bond formed between the heavy chain variable region and the light chain variable region. In some embodiments, a F(ab′) comprises a disulfide bond that increases stability of the F(ab′) molecule. In some embodiments, a F(ab′) comprises a disulfide bond that increases thermostability of the F(ab′) molecule.

In some embodiments, a KLRB1-binding agent is a bispecific antibody. Bispecific antibodies are capable of recognizing and binding at least two different antigens or epitopes. The different epitopes can either be within the same molecule (e.g., two epitopes on KLRB1) or on different molecules (e.g., one epitope on KLRB1 and one epitope on a different target). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., tissue) in a subject (e.g., a human). In some embodiments, a bispecific antibody has the ability to concentrate the actions of two agents to a common target (e.g., a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. In some embodiments, the bispecific antibodies comprise variant hinge regions incapable of forming disulfide linkages between identical heavy chains (e.g., reduce homodimer formation). In some embodiments, the bispecific antibodies comprise heavy chains with changes in amino acids that result in altered electrostatic interactions. In some embodiments, the bispecific antibodies comprise heavy chains with changes in amino acids that result in altered hydrophobic/hydrophilic interactions.

Bispecific antibodies can be intact antibodies or antibody fragments comprising antigen-binding sites.

KLRB1-binding agents with more than two valencies are also contemplated. In some embodiments, trispecific or tetraspecific antibodies are generated.

In some embodiments, a KLRB1-binding agent is an anti-KLRB1 antibody that comprises one, two, three, four, five, and/or six CDRs of any one of the antibodies described herein. In some embodiments, an anti-KLRB1 antibody comprises (i) one, two, and/or three heavy chain CDRs from antibody 10A3D6, e.g., as shown in Table 1, and/or (ii) one, two, and/or three light chain CDRs from antibody 10A3D6, e.g., as shown in Table 1. In some embodiments, an anti-KLRB1 antibody comprises (i) three heavy chain CDRs from antibody 10A3D6, e.g., as shown in Table 1 (i.e., a CDR1, a CDR2, and a CDR3), and (ii) three light chain CDRs from antibody 10A3D6, e.g., as shown in Table 1. In some embodiments, an anti-KLRB1 antibody comprises (i) one, two, and/or three heavy chain CDRs from antibody 10A3D6C109S, e.g., as shown in Table 2, and/or (ii) one, two, and/or three light chain CDRs from antibody 10A3D6C109S, e.g., as shown in Table 2. In some embodiments, an anti-KLRB1 antibody comprises (i) three heavy chain CDRs from antibody 10A3D6C109S, e.g., as shown in Table 2 (i.e., a CDR1, a CDR2, and a CDR3), and (ii) three light chain CDRs from antibody 10A3D6C109S, e.g., as shown in Table 2. In some embodiments, an anti-KLRB1 antibody comprises (i) one, two, and/or three heavy chain CDRs from antibody 10A3D6C109F, e.g., as shown in Table 3, and/or (ii) one, two, and/or three light chain CDRs from antibody 10A3D6C109F, e.g., as shown in Table 3. In some embodiments, an anti-KLRB1 antibody comprises (i) three heavy chain CDRs from antibody 10A3D6C109F, e.g., as shown in Table 3 (i.e., a CDR1, a CDR2, and a CDR3), and (ii) three light chain CDRs from antibody 10A3D6C109F, e.g., as shown in Table 3. In some embodiments, an anti-KLRB1 antibody comprises (i) one, two, and/or three heavy chain CDRs from antibody 10A3D6C109V, e.g., as shown in Table 4, and/or (ii) one, two, and/or three light chain CDRs from antibody 10A3D6humC109V, e.g., as shown in Table 4. In some embodiments, an anti-KLRB1 antibody comprises (i) three heavy chain CDRs from antibody 10A3D6humC109V, e.g., as shown in Table 4 (i.e., a CDR1, a CDR2, and a CDR3), and (ii) three light chain CDRs from antibody 10A3D6humC109V, e.g., as shown in Table 4.

In some embodiments, a KLRB1-binding agent is a humanized anti-KLRB1 antibody that comprises (i) one, two, and/or three heavy chain CDRs, and/or (ii) one, two, and/or three light chain CDRs from any one of Tables 1-53 or 5-53. In some embodiments, a KLRB1-binding agent is a humanized anti-KLRB1 antibody that comprises (i) three heavy chain CDRs (i.e., a CDR1, a CDR2, and a CDR3) and (ii) three light chain CDRs from any one of Tables 1-53 or 5-53, i.e., wherein the heavy chain CDRs and light chain CDRs are from the same table. In some embodiments, the heavy chains and light chains are from different tables, or the CDRs include CDRs from different tables.

In some embodiments, an anti-KLRB1 antibody comprises (i) one, two, and/or three heavy chain CDRs from antibody 10A3D6hum1.2, e.g., as shown in Table 30, and/or (ii) one, two, and/or three light chain CDRs from antibody 10A3D6hum1.2, e.g., as shown in Table 30. In some embodiments, an anti-KLRB1 antibody comprises (i) three heavy chain CDRs from antibody 10A3D6hum1.2, e.g., as shown in Table 30 (i.e., a CDR1, a CDR2, and a CDR3), and (ii) three light chain CDRs from antibody 10A3D6hum1.2, e.g., as shown in Table 30.

In some embodiments, an anti-KLRB1 antibody comprises (i) one, two, and/or three heavy chain CDRs from antibody 10A3D6hum1.5, e.g., as shown in Table 31, and/or (ii) one, two, and/or three light chain CDRs from antibody 10A3D6hum1.5, e.g., as shown in Table 31. In some embodiments, an anti-KLRB1 antibody comprises (i) three heavy chain CDRs from antibody 10A3D6hum1.5, e.g., as shown in Table 31 (i.e., a CDR1, a CDR2, and a CDR3), and (ii) three light chain CDRs from antibody 10A3D6hum1.5, e.g., as shown in Table 31.

In some embodiments, an anti-KLRB1 antibody comprises (i) one, two, and/or three heavy chain CDRs from antibody 10A3D6hum2.2, e.g., as shown in Table 32, and/or (ii) one, two, and/or three light chain CDRs from antibody 10A3D6hum2.2, e.g., as shown in Table 32. In some embodiments, an anti-KLRB1 antibody comprises (i) three heavy chain CDRs from antibody 10A3D6hum2.2, e.g., as shown in Table 32 (i.e., a CDR1, a CDR2, and a CDR3), and (ii) three light chain CDRs from antibody 10A3D6hum2.2, e.g., as shown in Table 32.

In some embodiments, an anti-KLRB1 antibody comprises (i) one, two, and/or three heavy chain CDRs from antibody 10A3D6hum8.1, e.g., as shown in Table 45, and/or (ii) one, two, and/or three light chain CDRs from antibody 10A3D6hum8.1, e.g., as shown in Table 45. In some embodiments, an anti-KLRB1 antibody comprises (i) three heavy chain CDRs from antibody 10A3D6hum8.1, e.g., as shown in Table 45 (i.e., a CDR1, a CDR2, and a CDR3), and (ii) three light chain CDRs from antibody 10A3D6hum8.1, e.g., as shown in Table 45.

In some embodiments, an anti-KLRB1 antibody comprises (i) one, two, and/or three heavy chain CDRs from antibody 10A3D6hum8.2, e.g., as shown in Table 46, and/or (ii) one, two, and/or three light chain CDRs from antibody 10A3D6hum8.2, e.g., as shown in Table 46. In some embodiments, an anti-KLRB1 antibody comprises (i) three heavy chain CDRs from antibody 10A3D6hum8.2, e.g., as shown in Table 46 (i.e., a CDR1, a CDR2, and a CDR3), and (ii) three light chain CDRs from antibody 10A3D6hum8.2, e.g., as shown in Table 46.

In some embodiments, an anti-KLRB1 antibody comprises (i) one, two, and/or three heavy chain CDRs from antibody 10A3D6hum8.3, e.g., as shown in Table 47, and/or (ii) one, two, and/or three light chain CDRs from antibody 10A3D6hum8.3, e.g., as shown in Table 47. In some embodiments, an anti-KLRB1 antibody comprises (i) three heavy chain CDRs from antibody 10A3D6hum8.3, e.g., as shown in Table 47 (i.e., a CDR1, a CDR2, and a CDR3), and (ii) three light chain CDRs from antibody 10A3D6hum8.3, e.g., as shown in Table 47.

In some embodiments, an anti-KLRB1 antibody comprises (i) one, two, and/or three heavy chain CDRs from antibody 10A3D6hum8.4, e.g., as shown in Table 48, and/or (ii) one, two, and/or three light chain CDRs from antibody 10A3D6hum8.4, e.g., as shown in Table 48. In some embodiments, an anti-KLRB1 antibody comprises (i) three heavy chain CDRs from antibody 10A3D6hum8.4, e.g., as shown in Table 48 (i.e., a CDR1, a CDR2, and a CDR3), and (ii) three light chain CDRs from antibody 10A3D6hum8.4, e.g., as shown in Table 48.

In some embodiments, an anti-KLRB1 antibody comprises (i) one, two, and/or three heavy chain CDRs from antibody 10A3D6hum8.5, e.g., as shown in Table 49, and/or (ii) one, two, and/or three light chain CDRs from antibody 10A3D6hum8.5, e.g., as shown in Table 49. In some embodiments, an anti-KLRB1 antibody comprises (i) three heavy chain CDRs from antibody 10A3D6hum8.5, e.g., as shown in Table 49 (i.e., a CDR1, a CDR2, and a CDR3), and (ii) three light chain CDRs from antibody 10A3D6hum8.5, e.g., as shown in Table 49.

In some embodiments, an anti-KLRB1 antibody comprises (i) one, two, and/or three heavy chain CDRs from antibody 10A3D6hum8.6, e.g., as shown in Table 50, and/or (ii) one, two, and/or three light chain CDRs from antibody 10A3D6hum8.6, e.g., as shown in Table 50. In some embodiments, an anti-KLRB1 antibody comprises (i) three heavy chain CDRs from antibody 10A3D6hum8.6, e.g., as shown in Table 50 (i.e., a CDR1, a CDR2, and a CDR3), and (ii) three light chain CDRs from antibody 10A3D6hum8.6, e.g., as shown in Table 50.

In some embodiments, an anti-KLRB1 antibody comprises (i) one, two, and/or three heavy chain CDRs from antibody 10A3D6hum8.7, e.g., as shown in Table 51, and/or (ii) one, two, and/or three light chain CDRs from antibody 10A3D6hum8.7, e.g., as shown in Table 51. In some embodiments, an anti-KLRB1 antibody comprises (i) three heavy chain CDRs from antibody 10A3D6hum8.7, e.g., as shown in Table 51 (i.e., a CDR1, a CDR2, and a CDR3), and (ii) three light chain CDRs from antibody 10A3D6hum8.7, e.g., as shown in Table 51.

In some embodiments, an anti-KLRB1 antibody comprises (i) one, two, and/or three heavy chain CDRs from antibody 10A3D6hum8.8, e.g., as shown in Table 52, and/or (ii) one, two, and/or three light chain CDRs from antibody 10A3D6hum8.8, e.g., as shown in Table 52. In some embodiments, an anti-KLRB1 antibody comprises (i) three heavy chain CDRs from antibody 10A3D6hum8.8, e.g., as shown in Table 52 (i.e., a CDR1, a CDR2, and a CDR3), and (ii) three light chain CDRs from antibody 10A3D6hum8.8, e.g., as shown in Table 52.

In some embodiments, an anti-KLRB1 antibody comprises (i) one, two, and/or three heavy chain CDRs from antibody 10A3D6hum8.9, e.g., as shown in Table 53, and/or (ii) one, two, and/or three light chain CDRs from antibody 10A3D6hum8.9, e.g., as shown in Table 53. In some embodiments, an anti-KLRB1 antibody comprises (i) three heavy chain CDRs from antibody 10A3D6hum8.9, e.g., as shown in Table 53 (i.e., a CDR1, a CDR2, and a CDR3), and (ii) three light chain CDRs from antibody 10A3D6hum8.9, e.g., as shown in Table 53.

TABLE 1
Antibody 10A3D6 Sequences

| 10A3D6 | Kabat | Chothia | AbM | Contact | IMGT |
|---|---|---|---|---|---|
| Heavy | GYTMN | GYSFTGY | GYSFTGYTMN | TGYTMN | GYSFTGYT |
| Chain | (SEQ ID NO: 3) | (SEQ ID NO: 9) | (SEQ ID NO: 11) | (SEQ ID NO: 13) | (SEQ ID NO: 19) |
| CDR1 | | | | | |
| Heavy | LINPNTGGTYYNQK | NPNTGG | LINPNTGGTY | WIGLINPNTGGTY | INPNTGGT |
| Chain | FKD | (SEQ ID NO: 10) | (SEQ ID NO: 12) | (SEQ ID NO: 14) | (SEQ ID NO: 20) |
| CDR2 | (SEQ ID NO: 4) | | | | |
| Heavy | LGDNYRGYFDC | LGDNYRGYFDC | LGDNYRGYFDC | ARLGDNYRGYFD | ARLGDNYRGYFDC |
| Chain | (SEQ ID NO: 5) | (SEQ ID NO: 5) | (SEQ ID NO: 5) | (SEQ ID NO: 15) | (SEQ ID NO: 21) |
| CDR3 | | | | | |
| Light | KASQDVGTAVV | KASQDVGTAVV | KASQDVGTAVV | GTAVVWY | QDVGTA |
| Chain | (SEQ ID NO: 6) | (SEQ ID NO: 6) | (SEQ ID NO: 6) | (SEQ ID NO: 16) | (SEQ ID NO: 22) |
| CDR1 | | | | | |
| Light | WASIRHT | WASIRHT | WASIRHT | VLIDWASIRH | WA |
| Chain | (SEQ ID NO: 7) | (SEQ ID NO: 7) | (SEQ ID NO: 7) | (SEQ ID NO: 17) | |
| CDR2 | | | | | |
| Light | QQYSTYLYT | QQYSTYLYT | QQYSTYLYT | QQYSTYLY | QQYSTYLYT |
| Chain | (SEQ ID NO: 8) | (SEQ ID NO: 8) | (SEQ ID NO: 8) | (SEQ ID NO: 18) | (SEQ ID NO: 8) |
| CDR3 | | | | | |

10A3D6 Heavy chain variable region (SEQ ID NO: 48)
EVQLQQSGPELVKPGASMKISCKASGYSFTGYTMNWVKQSHGKNLEWIGLINPNTGGTYYNQKFKDKATLTVDK
SSSTAYMELLSLTSEDSAVYYCARLGDNYRGYFDCWGQGTTLTVSS
10A3D6 Light chain variable region (SEQ ID NO: 49)
DIVMTQSHKFMSTSVGDRVNITCKASQDVGTAVVWYQQTPGQSPKVLIDWASIRHTGVPDRFTGSGSGTDFTLT
ISSVQSEDLADYFCQQYSTYLYTFGGGTKLEIK

TABLE 2
Antibody 10A3D6C109S Sequences

| 10A3D6C109S | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPNTGGT | NPNTGG | LINPNTGGTY | WIGLINPNTGGT | INPNTGGT |
| CDR2 | YYNQKFKD | (SEQ ID | (SEQ ID | Y (SEQ ID | (SEQ ID |
| | (SEQ ID | NO: 10) | NO: 12) | NO: 14) | NO: 20) |
| | NO: 4) | | | | |
| Heavy Chain | LGDNYRGYFDS | LGDNYRGYFD | LGDNYRGYFD | ARLGDNYRGYF | ARLGDNYRGYF |
| CDR3 | (SEQ ID | S (SEQ ID | S (SEQ ID | D (SEQ ID | DS (SEQ ID |
| | NO: 23) | NO: 23) | NO: 23) | NO: 15) | NO: 24) |
| Light Chain | KASQDVGTAVV | KASQDVGTAV | KASQDVGTAV | GTAVVWY | QDVGTA |
| CDR1 | (SEQ ID | V (SEQ ID | V (SEQ ID | (SEQ ID | (SEQ ID |
| | NO: 6) | NO: 6) | NO: 6) | NO: 16) | NO: 22) |
| Light Chain | WASIRHT | WASIRHT | WASIRHT | VLIDWASIRH | WA |
| CDR2 | (SEQ ID | (SEQ ID | (SEQ ID | (SEQ ID | |
| | NO: 7) | NO: 7) | NO: 7) | NO: 17) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6C109S Heavy chain variable region (SEQ ID NO: 50)
EVQLQQSGPELVKPGASMKISCKASGYSFTGYTMNWVKQSHGKNLEWIGLINPNTGGTYYNQKFKDKAT
LTVDKSSSTAYMELLSLTSEDSAVYYCARLGDNYRGYFDSWGQGTTLTVSS
10A3D6C109S Light chain variable region (SEQ ID NO: 49)
DIVMTQSHKFMSTSVGDRVNITCKASQDVGTAVVWYQQTPGQSPKVLIDWASIRHTGVPDRFTGSGSGT
DFTLTISSVQSEDLAD
YFCQQYSTYLYTFGGGTKLEIK

TABLE 3
Antibody 10A3D6C109F Sequences

| 10A3D6C109F | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID | (SEQ ID |
| | | NO: 9) | NO: 11) | NO: 13) | NO: 19) |
| Heavy Chain | LINPNTGGTYYNQ | NPNTGG | LINPNTGGTY | WIGLINPNTGG | INPNTGGT |
| CDR2 | KFKD | (SEQ ID | (SEQ ID | TY (SEQ ID | (SEQ ID |
| | (SEQ ID NO: 4) | NO: 10) | NO: 12) | NO: 14) | NO: 20) |
| Heavy Chain | LGDNYRGYFDF | LGDNYRGYFDF | LGDNYRGYFDF | ARLGDNYRGYFD | ARLGDNYRGYF |
| CDR3 | (SEQ ID NO: 25) | (SEQ ID | (SEQ ID | (SEQ ID | DF (SEQ ID |
| | | NO: 25) | NO: 25) | NO: 15) | NO: 26) |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID | (SEQ ID |
| | | NO: 6) | NO: 6) | NO: 16) | NO: 22) |
| Light Chain | WASIRHT | WASIRHT | WASIRHT | VLIDWASIRH | WA |
| CDR2 | (SEQ ID NO: 7) | (SEQ ID | (SEQ ID | (SEQ ID | |
| | | NO: 7) | NO: 7) | NO: 17) | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID | (SEQ ID |
| | | NO: 8) | NO: 8) | NO: 18) | NO: 8) |

10A3D6C109F Heavy chain variable region (SEQ ID NO: 51)
EVQLQQSGPELVKPGASMKISCKASGYSFTGYTMNWVKQSHGKNLEWIGLINPNTGGTYYNQKFKDKATLTVDK
SSSTAYMELLSLTSEDSAVYYCARLGDNYRGYFDFWGQGTTLTVSS
10A3D6C109F Light chain variable region (SEQ ID NO: 49)
DIVMTQSHKFMSTSVGDRVNITCKASQDVGTAVVWYQQTPGQSPKVLIDWASIRHTGVPDRFTGSGSGTDFTLT
ISSVQSEDLADYFCQQYSTYLYTFGGGTKLEIK

TABLE 4
Antibody 10A3D6C109V Sequences

| 10A3D6C109V | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID | (SEQ ID |
| | | NO: 9) | NO: 11) | NO: 13) | NO: 19) |
| Heavy Chain | LINPNTGGTYYNQKFK | NPNTGG | LINPNTGGTY | WIGLINPNTGG | INPNTGGT |
| CDR2 | D (SEQ ID NO: 4) | (SEQ ID | (SEQ ID | TY (SEQ ID | (SEQ ID |
| | | NO: 10) | NO: 12) | NO: 14) | NO: 20) |
| Heavy Chain | LGDNYRGYFDV | LGDNYRGYFDV | LGDNYRGYFDV | ARLGDNYRGYF | ARLGDNYRGYF |
| CDR3 | (SEQ ID NO: 27) | (SEQ ID | (SEQ ID | D (SEQ ID | DV (SEQ ID |
| | | NO: 27) | NO: 27) | NO: 15) | NO: 28) |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID | (SEQ ID |
| | | NO: 6) | NO: 6) | NO: 16) | NO: 22) |
| Light Chain | WASIRHT | WASIRHT | WASIRHT | VLIDWASIRH | WA |
| CDR2 | (SEQ ID NO: 7) | (SEQ ID | (SEQ ID | (SEQ ID | |
| | | NO: 7) | NO: 7) | NO: 17) | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID | (SEQ ID |
| | | NO: 8) | NO: 8) | NO: 18) | NO: 8) |

10A3D6C109V Heavy chain variable region (SEQ ID NO: 52)
EVQLQQSGPELVKPGASMKISCKASGYSFTGYTMNWVKQSHGKNLEWIGLINPNTGGTYYNQKFKDKATLTVDK
SSSTAYMELLSLTSEDSAVYYCARLGDNYRGYFDVWGQGTTLTVSS
10A3D6C109V Light chain variable region (SEQ ID NO: 49)
DIVMTQSHKFMSTSVGDRVNITCKASQDVGTAVVWYQQTPGQSPKVLIDWASIRHTGVPDRFTGSGSGTDFTLT
ISSVQSEDLADYFCQQYSTYLYTFGGGTKLEIK

TABLE 5
Antibody 10A3D6C109Chum1.1 Sequences

| 10A3D6C109Chum1.1 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID NO: 13) | (SEQ ID NO: 19) |
| | | NO: 9) | NO: 11) | | |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID | (SEQ ID | (SEQ ID NO: 29) | (SEQ ID NO: 20) |
| | NO: 4) | NO: 10) | NO: 12) | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID | (SEQ ID | (SEQ ID NO: 15) | (SEQ ID NO: 21) |
| | | NO: 5) | NO: 5) | | |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID NO: 16) | (SEQ ID NO: 22) |
| | | NO: 6) | NO: 6) | | |
| Light Chain | WASIRHT | WASIRHT | WASIRHT | LLIYWASIRH | WA |
| CDR2 | (SEQ ID NO: 7) | (SEQ ID | (SEQ ID | (SEQ ID NO: 76) | |
| | | NO: 7) | NO: 7) | | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID NO: 18) | (SEQ ID NO: 8) |
| | | NO: 8) | NO: 8) | | |

10A3D6C109Chum1.1 Heavy chain variable region (SEQ ID NO: 53)
QVQLVQSGAEVKKPGASVKVSCKASGYSFTGYTMNWVRQAPGQGLEWMGLINPNTGGTYYNQKFKDRVTMTRD
TSISTAYMELSRLRSDDTAVYYCARLGDNYRGYFDCWGQGTTVTVSS
10A3D6C109Chum1.1 Light chain variable region (SEQ ID NO: 54)
DIQLTQSPSFLSASVGDRVTITCKASQDVGTAVVWYQQKPGKAPKLLIYWASIRHTGVPSRFSGSGSGTEFTLT
ISSLQPEDFATYYCQQYSTYLYTFGGGTKLEIK

TABLE 6
Antibody 10A3D6C109Chum1.2 Sequences

| 10A3D6C109Chum1.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID NO: 13) | (SEQ ID NO: 19) |
| | | NO: 9) | NO: 11) | | |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID | (SEQ ID | (SEQ ID NO: 29) | (SEQ ID NO: 20) |
| | NO: 4) | NO: 10) | NO: 12) | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID | (SEQ ID | (SEQ ID NO: 15) | (SEQ ID NO: 21) |
| | | NO: 5) | NO: 5) | | |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID NO: 16) | (SEQ ID NO: 22) |
| | | NO: 6) | NO: 6) | | |
| Light Chain | WASIRHT | WASIRHT | WASIRHT | LLIDWASIRH | WA |
| CDR2 | (SEQ ID NO: 7) | (SEQ ID | (SEQ ID | (SEQ ID NO: 30) | |
| | | NO: 7) | NO: 7) | | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID NO: 18) | (SEQ ID NO: 8) |
| | | NO: 8) | NO: 8) | | |

10A3D6C109Chum1.2 Heavy chain variable region (SEQ ID NO: 53)
QVQLVQSGAEVKKPGASVKVSCKASGYSFTGYTMNWVRQAPGQGLEWMGLINPNTGGTYYNQKFKDRVTMTRDT
SISTAYMELSRLRSDDTAVYYCARLGDNYRGYFDCWGQGTTVTVSS
10A3D6C109Chum1.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLT
ISSLQPEDFADYFCQQYSTYLYTFGGGTKLEIK

TABLE 7
Antibody 10A3D6C109Chum1.3 Sequences

| 10A3D6C109Chum1.3 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID | (SEQ ID |
| | | NO: 9) | NO: 11) | NO: 13) | NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID | (SEQ ID | (SEQ ID | (SEQ ID |
| | NO: 4) | NO: 10) | NO: 12) | NO: 29) | NO: 20) |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID | (SEQ ID | (SEQ ID | (SEQ ID |
| | | NO: 5) | NO: 5) | NO: 15) | NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID | (SEQ ID |
| | | NO: 6) | NO: 6) | NO: 16) | NO: 22) |
| Light Chain | WASIRHT | WASIRHT | WASIRHT | LLIDWASIRH | WA |
| CDR2 | (SEQ ID NO: 7) | (SEQ ID | (SEQ ID | (SEQ ID | |
| | | NO: 7) | NO: 7) | NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID | (SEQ ID |
| | | NO: 8) | NO: 8) | NO: 18) | NO: 8) |

10A3D6C109Chum1.3 Heavy chain variable region (SEQ ID NO: 53)
QVQLVQSGAEVKKPGASVKVSCKASGYSFTGYTMNWVRQAPGQGLEWMGLINPNTGGTYYNQKFKDRVTMTRDTS
ISTAYMELSRLRSDDTAVYYCARLGDNYRGYFDCWGQGTTVTVSS
10A3D6C109Chum1.3 Light chain variable region (SEQ ID NO: 56)
DIQLTQSPSFLSASVGDRVTITCKASQDVGTAVVWYQQTPGKAPKLLIDWASIRHTGVPSRFSGSGSGTEFTLTI
SSLQPEDFADYFCQQYSTYLYTFGGGTKLEIK

TABLE 8
Antibody 10A3D6C109Chum1.4 Sequences

| 10A3D6C109Chum1.4 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID NO: 13) | (SEQ ID NO: 19) |
| | | NO: 9) | NO: 11) | | |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID | (SEQ ID | (SEQ ID NO: 29) | (SEQ ID NO: 20) |
| | NO: 4) | NO: 10) | NO: 12) | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID | (SEQ ID | (SEQ ID NO: 15) | (SEQ ID NO: 21) |
| | | NO: 5) | NO: 5) | | |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID NO: 16) | (SEQ ID NO: 22) |
| | | NO: 6) | NO: 6) | | |
| Light Chain | WASIRHT | WASIRHT | WASIRHT | LLIDWASIRH | WA |
| CDR2 | (SEQ ID NO: 7) | (SEQ ID | (SEQ ID | (SEQ ID NO: 30) | |
| | | NO: 7) | NO: 7) | | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID NO: 18) | (SEQ ID NO: 8) |
| | | NO: 8) | NO: 8) | | |

10A3D6C109Chum1.4 Heavy chain variable region (SEQ ID NO: 53)
QVQLVQSGAEVKKPGASVKVSCKASGYSFTGYTMNWVRQAPGQGLEWMGLINPNTGGTYYNQKFKDRVTMTRDT
SISTAYMELSRLRSDDTAVYYCARLGDNYRGYFDCWGQGTTVTVSS
10A3D6C109Chum1.4 Light chain variable region (SEQ ID NO: 57)
DIQLTQSPSFLSASVGDRVTITCKASQDVGTAVVWYQQTPGKAPKLLIDWASIRHTGVPDRFSGSGSGTEFTLT
ISSLQPEDFADYFCQQYSTYLYTFGGGTKLEIK

TABLE 9
Antibody 10A3D6C109Chum1.5 Sequences

| 10A3D6C109Chum1.5 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum1.5 Heavy chain variable region (SEQ ID NO: 53)
QVQLVQSGAEVKKPGASVKVSCKASGYSFTGYTMNWVRQAPGQGLEWMGLINPNTGGTYYNQKFKDRVTMTRDT
SISTAYMELSRLRSDDTAVYYCARLGDNYRGYFDCWGQGTTVTVSS
10A3D6C109Chum1.5 Light chain variable region (SEQ ID NO: 58)
EIVMTQSPATLSVSPGERATLSCKASQDVGTAVVWYQQKPGQAPRLLIDWASIRHTGIPARFSGSGSGTEFTLT
ISSLQSEDFAVYYCQQYSTYLYTFGGGTKVEIK

TABLE 10
Antibody 10A3D6C109Chum2.1 Sequences

| 10A3D6C109Chum2.1 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIYWASIRH | WA |
| CDR2 | (SEQ ID NO: 7) | (SEQ ID NO: 7) | (SEQ ID NO: 7) | (SEQ ID NO: 76) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum2.1 Heavy chain variable region (SEQ ID NO: 59)
QVQLVQSGAEVKKPGASVKVSCKASGYSFTGYTMNWVRQAPGQNLEWIGLINPNTGGTYYNQKFKDRVTLTVDT
SISTAYMELSRLRSDDTAVYYCARLGDNYRGYFDCWGQGTTVTVSS
10A3D6C109Chum2.1 Light chain variable region (SEQ ID NO: 54)
DIQLTQSPSFLSASVGDRVTITCKASQDVGTAVVWYQQKPGKAPKLLIYWASIRHTGVPSRFSGSGSGTEFTLT
ISSLQPEDFATYYCQQYSTYLYTFGGGTKLEIK

TABLE 11
Antibody 10A3D6C109Chum2.2 Sequences

| 10A3D6C109Chum2.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID NO: 13) | (SEQ ID NO: 19) |
| | | NO: 9) | NO: 11) | | |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID | (SEQ ID | (SEQ ID NO: 29) | (SEQ ID NO: 20) |
| | NO: 4) | NO: 10) | NO: 12) | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID | (SEQ ID | (SEQ ID NO: 15) | (SEQ ID NO: 21) |
| | | NO: 5) | NO: 5) | | |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID NO: 16) | (SEQ ID NO: 22) |
| | | NO: 6) | NO: 6) | | |
| Light Chain | WASIRHT | WASIRHT | WASIRHT | LLIDWASIRH | WA |
| CDR2 | (SEQ ID NO: 7) | (SEQ ID | (SEQ ID | (SEQ ID NO: 30) | |
| | | NO: 7) | NO: 7) | | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID NO: 18) | (SEQ ID NO: 8) |
| | | NO: 8) | NO: 8) | | |

10A3D6C109Chum2.2 Heavy chain variable region (SEQ ID NO: 59)
QVQLVQSGAEVKKPGASVKVSCKASGYSFTGYTMNWVRQAPGQNLEWIGLINPNTGGTYYNQKFKDRVTLTVDTS
ISTAYMELSRLRSDDTAVYYCARLGDNYRGYFDCWGQGTTVTVSS
10A3D6C109Chum2.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTI
SSLQPEDFADYFCQQYSTYLYTFGGGTKLEIK

TABLE 12
Antibody 10A3D6C109Chum2.3 Sequences

| 10A3D6C109Chum2.3 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID | (SEQ ID | (SEQ ID NO: 13) | (SEQ ID NO: 19) |
| | | NO: 9) | NO: 11) | | |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID | (SEQ ID | (SEQ ID NO: 29) | (SEQ ID NO: 20) |
| | NO: 4) | NO: 10) | NO: 12) | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID | (SEQ ID | (SEQ ID NO: 15) | (SEQ ID NO: 21) |
| | | NO: 5) | NO: 5) | | |
| Light Chain | KASQDVGTAVV | KASQDVGTAVV | KASQDVGTAVV | GTAVVWY | QDVGTA |
| CDR1 | (SEQ ID NO: 6) | (SEQ ID | (SEQ ID | (SEQ ID NO: 16) | (SEQ ID NO: 22) |
| | | NO: 6) | NO: 6) | | |
| Light Chain | WASIRHT | WASIRHT | WASIRHT | LLIDWASIRH | WA |
| CDR2 | (SEQ ID NO: 7) | (SEQ ID | (SEQ ID | (SEQ ID NO: 30) | |
| | | NO: 7) | NO: 7) | | |
| Light Chain | QQYSTYLYT | QQYSTYLYT | QQYSTYLYT | QQYSTYLY | QQYSTYLYT |
| CDR3 | (SEQ ID NO: 8) | (SEQ ID | (SEQ ID | (SEQ ID NO: 18) | (SEQ ID NO: 8) |
| | | NO: 8) | NO: 8) | | |

10A3D6C109Chum2.3 Heavy chain variable region (SEQ ID NO: 59)
QVQLVQSGAEVKKPGASVKVSCKASGYSFTGYTMNWVRQAPGQNLEWIGLINPNTGGTYYNQKFKDRVTLTVDTS
ISTAYMELSRLRSDDTAVYYCARLGDNYRGYFDCWGQGTTVTVSS
10A3D6C109Chum2.3 Light chain variable region (SEQ ID NO: 56)
DIQLTQSPSFLSASVGDRVTITCKASQDVGTAVVWYQQTPGKAPKLLIDWASIRHTGVPSRFSGSGSGTEFTLTI
SSLQPEDFADYFCQQYSTYLYTFGGGTKLEIK

TABLE 13
Antibody 10A3D6C109Chum2.4 Sequences

| 10A3D6C109C | | | | | |
|---|---|---|---|---|---|
| hum2.4 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum2.4 Heavy chain variable region (SEQ ID NO: 59)
QVQLVQSGAEVKKPGASVKVSCKASGYSFTGYTMNWVRQAPGQNLEWIGLINPNTGGTYYNQKFKDRVTLTVDTSISTAYMELSRL
RSDDTAVYYCARLGDNYRGYFDCWGQGTTVTVSS
10A3D6C109Chum2.4 Light chain variable region (SEQ ID NO: 57)
DIQLTQSPSFLSASVGDRVTITCKASQDVGTAVVWYQQTPGKAPKLLIDWASIRHTGVPDRFSGSGSGTEFTLTISSLQPEDFADY
FCQQYSTYLYTFGGGTKLEIK

TABLE 14
Antibody 10A3D6C109Chum2.5 Sequences

| 10A3D6C109C | | | | | |
|---|---|---|---|---|---|
| hum2.5 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum2.5 Heavy chain variable region (SEQ ID NO: 59)
QVQLVQSGAEVKKPGASVKVSCKASGYSFTGYTMNWVRQAPGQNLEWIGLINPNTGGTYYNQKFKDRVTLTVDTSISTAYMELSRL
RSDDTAVYYCARLGDNYRGYFDCWGQGTTVTVSS
10A3D6C109Chum2.5 Light chain variable region (SEQ ID NO: 58)
EIVMTQSPATLSVSPGERATLSCKASQDVGTAVVWYQQKPGQAPRLLIDWASIRHTGIPARFSGSGSGTEFTLTISSLQSEDFAVY
YCQQYSTYLYTFGGGTKVEIK

TABLE 15
Antibody 10A3D6C109Chum3.1 Sequences

| 10A3D6C109C | | | | | |
|---|---|---|---|---|---|
| hum3.1 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIYWASIRH | WA |
| CDR2 | (SEQ ID NO: 7) | (SEQ ID NO: 7) | (SEQ ID NO: 7) | (SEQ ID NO: 76) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum3.1 Heavy chain variable region (SEQ ID NO: 60)
QVQLVQSGAEVKKPGASVKVSCKASGYSFTGYTMNWVRQAHGQNLEWIGLINPNTGGTYYNQKFKDRVTLTVDTSISTAYMELSRL
RSDDTAVYYCARLGDNYRGYFDCWGQGTTVTVSS
10A3D6C109Chum3.1 Light chain variable region (SEQ ID NO: 54)
DIQLTQSPSFLSASVGDRVTITCKASQDVGTAVVWYQQKPGKAPKLLIYWASIRHTGVPSRFSGSGSGTEFTLTISSLQPEDFATYY
CQQYSTYLYTFGGGTKLEIK

TABLE 16
Antibody 10A3D6C109Chum3.2 Sequences

| 10A3D6C109C | | | | | |
|---|---|---|---|---|---|
| hum3.2 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum3.2 Heavy chain variable region (SEQ ID NO: 60)
QVQLVQSGAEVKKPGASVKVSCKASGYSFTGYTMNWVRQAHGQNLEWIGLINPNTGGTYYNQKFKDRVTLTVDTSISTAYMELSRL
RSDDTAVYYCARLGDNYRGYFDCWGQGTTVTVSS
10A3D6C109Chum3.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISSLQPEDFADY
FCQQYSTYLYTFGGGTKLEIK

TABLE 17
Antibody 10A3D6C109Chum3.3 Sequences

| 10A3D6C109C | | | | | |
|---|---|---|---|---|---|
| hum3.3 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum3.3 Heavy chain variable region (SEQ ID NO: 60)
QVQLVQSGAEVKKPGASVKVSCKASGYSFTGYTMNWVRQAHGQNLEWIGLINPNTGGTYYNQKFKDRVTLTVDTSISTAYMELSRL
RSDDTAVYYCARLGDNYRGYFDCWGQGTTVTVSS
10A3D6C109Chum3.3 Light chain variable region (SEQ ID NO: 56)
DIQLTQSPSFLSASVGDRVTITCKASQDVGTAVVWYQQTPGKAPKLLIDWASIRHTGVPSRFSGSGSGTEFTLTISSLQPEDFADY
FCQQYSTYLYTFGGGTKLEIK

TABLE 18
Antibody 10A3D6C109Chum3.4 Sequences

| 10A3D6C109C | | | | | |
|---|---|---|---|---|---|
| hum3.4 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum3.4 Heavy chain variable region (SEQ ID NO: 60)
QVQLVQSGAEVKKPGASVKVSCKASGYSFTGYTMNWVRQAHGQNLEWIGLINPNTGGTYYNQKFKDRVTLTVDTSISTAYMELSRL
RSDDTAVYYCARLGDNYRGYFDCWGQGTTVTVSS
10A3D6C109Chum3.4 Light chain variable region (SEQ ID NO: 57)
DIQLTQSPSFLSASVGDRVTITCKASQDVGTAVVWYQQTPGKAPKLLIDWASIRHTGVPDRFSGSGSGTEFTLTISSLQPEDFADY
FCQQYSTYLYTFGGGTKLEIK

TABLE 19
Antibody 10A3D6C109Chum3.5 Sequences

| 10A3D6C109C | | | | | |
|---|---|---|---|---|---|
| hum3.5 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum3.5 Heavy chain variable region (SEQ ID NO: 60)
QVQLVQSGAEVKKPGASVKVSCKASGYSFTGYTMNWVRQAHGQNLEWIGLINPNTGGTYYNQKFKDRVTLTVDTSISTAYMELSRL
RSDDTAVYYCARLGDNYRGYFDCWGQGTTVTVSS
10A3D6C109Chum3.5 Light chain variable region (SEQ ID NO: 58)
EIVMTQSPATLSVSPGERATLSCKASQDVGTAVVWYQQKPGQAPRLLIDWASIRHTGIPARFSGSGSGTEFTLTISSLQSEDFAVY
YCQQYSTYLYTFGGGTKVEIK

TABLE 20
Antibody 10A3D6C109Chum4.1 Sequences

| 10A3D6C109C | | | | | |
|---|---|---|---|---|---|
| hum4.1 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIYWASIRH | WA |
| CDR2 | (SEQ ID NO: 7) | (SEQ ID NO: 7) | (SEQ ID NO: 7) | (SEQ ID NO: 76) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum4.1 Heavy chain variable region (SEQ ID NO: 61)
QVQLVQSGAEVKKPGASVKVSCKASGYSFTGYTMNWVRQAHGQNLEWIGLINPNTGGTYYNQKFKDRATLTVDTSISTAYMELSRL
RSDDTAVYYCARLGDNYRGYFDCWGQGTTVTVSS
10A3D6C109Chum4.1 Light chain variable region (SEQ ID NO: 54)
DIQLTQSPSFLSASVGDRVTITCKASQDVGTAVVWYQQKPGKAPKLLIYWASIRHTGVPSRFSGSGSGTEFTLTISSLQPEDFATY
YCQQYSTYLYTFGGGTKLEIK

TABLE 21
Antibody 10A3D6C109Chum4.2 Sequences

| 10A3D6C109C | | | | | |
|---|---|---|---|---|---|
| hum4.2 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum4.2 Heavy chain variable region (SEQ ID NO: 61)
QVQLVQSGAEVKKPGASVKVSCKASGYSFTGYTMNWVRQAHGQNLEWIGLINPNTGGTYYNQKFKDRATLTVDTSISTAYMELSRL
RSDDTAVYYCARLGDNYRGYFDCWGQGTTVTVSS
10A3D6C109Chum4.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISSLQPEDFADY
FCQQYSTYLYTFGGGTKLEIK

TABLE 22
Antibody 10A3D6C109Chum4.3 Sequences

| 10A3D6C109C | | | | | |
|---|---|---|---|---|---|
| hum4.3 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum4.3 Heavy chain variable region (SEQ ID NO: 61)
QVQLVQSGAEVKKPGASVKVSCKASGYSFTGYTMNWVRQAHGQNLEWIGLINPNTGGTYYNQKFKDRATLTVDTSISTAYMELSRL
RSDDTAVYYCARLGDNYRGYFDCWGQGTTVTVSS
10A3D6C109Chum4.3 Light chain variable region (SEQ ID NO: 56)
DIQLTQSPSFLSASVGDRVTITCKASQDVGTAVVWYQQTPGKAPKLLIDWASIRHTGVPSRFSGSGSGTEFTLTISSLQPEDFADY
FCQQYSTYLYTFGGGTKLEIK

TABLE 23
Antibody 10A3D6C109Chum4.4 Sequences

| 10A3D6C109C | | | | | |
|---|---|---|---|---|---|
| hum4.4 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum4.4 Heavy chain variable region (SEQ ID NO: 61)
QVQLVQSGAEVKKPGASVKVSCKASGYSFTGYTMNWVRQAHGQNLEWIGLINPNTGGTYYNQKFKDRATLTVDTSISTAYMELSRL
RSDDTAVYYCARLGDNYRGYFDCWGQGTTVTVSS
10A3D6C109Chum4.4 Light chain variable region (SEQ ID NO: 57)
DIQLTQSPSFLSASVGDRVTITCKASQDVGTAVVWYQQTPGKAPKLLIDWASIRHTGVPDRFSGSGSGTEFTLTISSLQPEDFADY
FCQQYSTYLYTFGGGTKLEIK

TABLE 24
Antibody 10A3D6C109Chum4.5 Sequences

| 10A3D6C109C | | | | | |
|---|---|---|---|---|---|
| hum4.5 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum4.5 Heavy chain variable region (SEQ ID NO: 61)
QVQLVQSGAEVKKPGASVKVSCKASGYSFTGYTMNWVRQAHGQNLEWIGLINPNTGGTYYNQKFKDRATLTVDTSISTAYMELSRL
RSDDTAVYYCARLGDNYRGYFDCWGQGTTVTVSS
10A3D6C109Chum4.5 Light chain variable region (SEQ ID NO: 58)
EIVMTQSPATLSVSPGERATLSCKASQDVGTAVVWYQQKPGQAPRLLIDWASIRHTGIPARFSGSGSGTEFTLTISSLQSEDFAVY
YCQQYSTYLYTFGGGTKVEIK

TABLE 25
Antibody 10A3D6C109Chum5.1 Sequences

| 10A3D6C109C | | | | | |
|---|---|---|---|---|---|
| hum5.1 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIYWASIRH | WA |
| CDR2 | (SEQ ID NO: 7) | (SEQ ID NO: 7) | (SEQ ID NO: 7) | (SEQ ID NO: 76) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum5.1 Heavy chain variable region (SEQ ID NO: 62)
QVQLVQSGAEVKKPGASVKVSCKASGYSFTGYTMNWVRQAPGQNLEWMGLINPNTGGTYYNQKFKDRVTLTVDTSISTAYMELSR
LRSDDTVVYYCARLGDNYRGYFDCWGQGTLVTVSS
10A3D6C109Chum5.1 Light chain variable region (SEQ ID NO: 54)
DIQLTQSPSFLSASVGDRVTITCKASQDVGTAVVWYQQKPGKAPKLLIYWASIRHTGVPSRFSGSGSGTEFTLTISSLQPEDFAT
YYCQQYSTYLYTFGGGTKLEIK

TABLE 26
Antibody 10A3D6C109Chum5.2 Sequences

| 10A3D6C109C | | | | | |
|---|---|---|---|---|---|
| hum5.2 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum5.2 Heavy chain variable region (SEQ ID NO: 62)
QVQLVQSGAEVKKPGASVKVSCKASGYSFTGYTMNWVRQAPGQNLEWMGLINPNTGGTYYNQKFKDRVTLTVDTSISTAYMELSR
LRSDDTVVYYCARLGDNYRGYFDCWGQGTLVTVSS
10A3D6C109Chum5.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISSLQPEDFAD
YFCQQYSTYLYTFGGGTKLEIK

TABLE 27
Antibody 10A3D6C109Chum5.3 Sequences

| 10A3D6C109C | | | | | |
|---|---|---|---|---|---|
| hum5.3 | Kabat | Chothia | AbM | Contact | IMGT |
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum5.3 Heavy chain variable region (SEQ ID NO: 62)
QVQLVQSGAEVKKPGASVKVSCKASGYSFTGYTMNWVRQAPGQNLEWMGLINPNTGGTYYNQKFKDRVTLTVDTSISTAYMELSR
LRSDDTVVYYCARLGDNYRGYFDCWGQGTLVTVSS
10A3D6C109Chum5.3 Light chain variable region (SEQ ID NO: 56)
DIQLTQSPSFLSASVGDRVTITCKASQDVGTAVVWYQQTPGKAPKLLIDWASIRHTGVPSRFSGSGSGTEFTLTISSLQPEDFAD
YFCQQYSTYLYTFGGGTKLEIK

TABLE 28
Antibody 10A3D6C109Chum5.4 Sequences

| 10A3D6C109Chum5.4 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum5.4 Heavy chain variable region (SEQ ID NO: 62)
QVQLVQSGAEVKKPGASVKVSCKASGYSFTGYTMNWVRQAPGQNLEWMGLINPNTGGTYYNQKFKDRVTLTVDTSISTAYMELSR
LRSDDTVVYYCARLGDNYRGYFDCWGQGTLVTVSS
10A3D6C109Chum5.4 Light chain variable region (SEQ ID NO: 57)
DIQLTQSPSFLSASVGDRVTITCKASQDVGTAVVWYQQTPGKAPKLLIDWASIRHTGVPDRFSGSGSGTEFTLTISSLQPEDFADYFCQQ
YSTYLYTFGGGTKLEIK

TABLE 29
Antibody 10A3D6C109Chum5.5 Sequences

| 10A3D6C109Chum5.5 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDC | LGDNYRGYFDC | LGDNYRGYFDC | ARLGDNYRGYFD | ARLGDNYRGYFDC |
| CDR3 | (SEQ ID NO: 5) | (SEQ ID NO: 5) | (SEQ ID NO: 5) | (SEQ ID NO: 15) | (SEQ ID NO: 21) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6C109Chum5.5 Heavy chain variable region (SEQ ID NO: 62)
QVQLVQSGAEVKKPGASVKVSCKASGYSFTGYTMNWVRQAPGQNLEWMGLINPNTGGTYYNQKFKDRVTLTVDTSISTAYMELSR
LRSDDTVVYYCARLGDNYRGYFDCWGQGTLVTVSS
10A3D6C109Chum5.5 Light chain variable region (SEQ ID NO: 58)
EIVMTQSPATLSVSPGERATLSCKASQDVGTAVVWYQQKPGQAPRLLIDWASIRHTGIPARFSGSGSGTEFTLTISSLQSEDFAVYYCQ
QYSTYLYTFGGGTKVEIK

TABLE 30
Antibody 10A3D6hum1.2 Sequences

| 10A3D6hum1.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDS | LGDNYRGYFDS | LGDNYRGYFDS | ARLGDNYRGYFD | ARLGDNYRGYFDS |
| CDR3 | (SEQ ID NO: 23) | (SEQ ID NO: 23) | (SEQ ID NO: 23) | (SEQ ID NO: 15) | (SEQ ID NO: 24) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum1.2 Heavy chain variable region (SEQ ID NO: 63)
QVQLVQSGAEVKKPGASVKVSCKASGYSFTGYTMNWVRQAPGQGLEWMGLINPNTGGTYYNQKFKDRVTMTRDTSISTAYMELSR
LRSDDTAVYYCARLGDNYRGYFDSWGQGTTVTVSS
10A3D6hum1.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISSLQPEDFADYFCQQ
YSTYLYTFGGGTKLEIK

TABLE 31
Antibody 10A3D6hum1.5 Sequences

| 10A3D6hum1.5 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WMGLINPNTGGTY | INPNTGGT |
| CDR2 | KFKD (SEQ ID | (SEQ ID NO: 10) | (SEQ ID NO: 12) | (SEQ ID NO: 29) | (SEQ ID NO: 20) |
| | NO: 4) | | | | |
| Heavy Chain | LGDNYRGYFDS | LGDNYRGYFDS | LGDNYRGYFDS | ARLGDNYRGYFD | ARLGDNYRGYFDS |
| CDR3 | (SEQ ID NO: 23) | (SEQ ID NO: 23) | (SEQ ID NO: 23) | (SEQ ID NO: 15) | (SEQ ID NO: 24) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum1.5 Heavy chain variable region (SEQ ID NO: 63)
QVQLVQSGAEVKKPGASVKVSCKASGYSFTGYTMNWVRQAPGQGLEWMGLINPNTGGTYYNQKFKDRVTMTRDTSISTAYMELSR
LRSDDTAVYYCARLGDNYRGYFDSWGQGTTVTVSS
10A3D6hum1.5 Light chain variable region (SEQ ID NO: 58)
EIVMTQSPATLSVSPGERATLSCKASQDVGTAVVWYQQKPGQAPRLLIDWASIRHTGIPARFSGSGSGTEFTLTISSLQSEDFAVYYCQ
QYSTYLYTFGGGTKVEIK

TABLE 32
Antibody 10A3D6hum2.2 Sequences

| 10A3D6hum2.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD (SEQ ID | (SEQ ID NO: 10) | (SEQ ID NO: 12) | (SEQ ID NO: 14) | (SEQ ID NO: 20) |
| | NO: 4) | | | | |
| Heavy Chain | LGDNYRGYFDS | LGDNYRGYFDS | LGDNYRGYFDS | ARLGDNYRGYFD | ARLGDNYRGYFDS |
| CDR3 | (SEQ ID NO: 23) | (SEQ ID NO: 23) | (SEQ ID NO: 23) | (SEQ ID NO: 15) | (SEQ ID NO: 24) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum2.2 Heavy chain variable region (SEQ ID NO: 64)
QVQLVQSGAEVKKPGASVKVSCKASGYSFTGYTMNWVRQAPGQNLEWIGLINPNTGGTYYNQKFKDRVTLTVDTSISTAYMELSRL
RSDDTAVYYCARLGDNYRGYFDSWGQGTTVTVSS
10A3D6hum2.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISSLQPEDFADYFCQQ
YSTYLYTFGGGTKLEIK

TABLE 33
Antibody 10A3D6hum6.1 Sequences

| 10A3D6hum6.1 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD (SEQ ID | (SEQ ID NO: 10) | (SEQ ID NO: 12) | (SEQ ID NO: 14) | (SEQ ID NO: 20) |
| | NO: 4) | | | | |
| Heavy Chain | LGDNYRGYFDA | LGDNYRGYFDA | LGDNYRGYFDA | ARLGDNYRGYFD | ARLGDNYRGYFDA |
| CDR3 | (SEQ ID NO: 31) | (SEQ ID NO: 31) | (SEQ ID NO: 31) | (SEQ ID NO: 15) | (SEQ ID NO: 32) |
| Light Chain | RASQDVGTAVV | RASQDVGTAVV | RASQDVGTAVV | GTAVVWY | QDVGTA |
| CDR1 | (SEQ ID NO: 32) | (SEQ ID NO: 32) | (SEQ ID NO: 32) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum6.1 Heavy chain variable region (SEQ ID NO: 65)
QVQLVQSGAEVKKPGASVKVSCKASGYSFTGYTMNWVRQAPGQGLEWIGLINPNTGGTYYNQKFKDRVTLTVDTSISTAYMELSRL
RSDDTAVYYCARLGDNYRGYFDAWGQGTTVTVSS
10A3D6hum6.1 Light chain variable region (SEQ ID NO: 66)
DIQLTQSPSFLSASVGDRVTITCRASQDVGTAVVWYQQKPGKAPKLLIDWASIRHTGVPSRFSGSGSGTEFTLTISSLQPEDFATYYCQQ
YSTYLYTFGGGTKLEIK

TABLE 34
Antibody 10A3D6hum6.2 Sequences

| 10A3D6hum6.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD (SEQ ID | (SEQ ID NO: 10) | (SEQ ID NO: 12) | (SEQ ID NO: 14) | (SEQ ID NO: 20) |
| | NO: 4) | | | | |
| Heavy Chain | LGDNYRGYFDA | LGDNYRGYFDA | LGDNYRGYFDA | ARLGDNYRGYFD | ARLGDNYRGYFDA |
| CDR3 | (SEQ ID NO: 31) | (SEQ ID NO: 31) | (SEQ ID NO: 31) | (SEQ ID NO: 15) | (SEQ ID NO: 32) |
| Light Chain | RASQDVGSAVV | RASQDVGSAVV | RASQDVGSAVV | GSAVVWY | QDVGSA |
| CDR1 | (SEQ ID NO: 33) | (SEQ ID NO: 33) | (SEQ ID NO: 33) | (SEQ ID NO: 34) | (SEQ ID NO: 35) |
| Light Chain | WASIRHS | WASIRHS | WASIRHS | LLIDWASIRH | WA |
| CDR2 | (SEQ ID NO: 36) | (SEQ ID NO: 36) | (SEQ ID NO: 36) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum6.2 Heavy chain variable region (SEQ ID NO: 65)
QVQLVQSGAEVKKPGASVKVSCKASGYSFTGYTMNWVRQAPGQGLEWIGLINPNTGGTYYNQKFKDRVTLTVDTSISTAYMELSRL
RSDDTAVYYCARLGDNYRGYFDAWGQGTTVTVSS
10A3D6hum6.2 Light chain variable region (SEQ ID NO: 67)
DIQLTQSPSFLSASVGDRVTITCRASQDVGSAVVWYQQKPGKAPKLLIDWASIRHSGVPSRFSGSGSGTEFTLTISSLQPEDFATYYCQQ
YSTYLYTFGGGTKLEIK

TABLE 35
Antibody 10A3D6hum6.3 Sequences

| 10A3D6hum6.3 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPATGGTYYNQ | NPATGG (SEQ ID | LINPATGGTY | WIGLINPATGGTY | INPATGGT |
| CDR2 | KFKD | NO: 38) | (SEQ ID NO: 39) | (SEQ ID NO: 40) | (SEQ ID NO: 41) |
| | (SEQ ID NO: 37) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID NO: 42) | (SEQ ID NO: 42) | (SEQ ID NO: 42) | (SEQ ID NO: 43) | (SEQ ID NO: 44) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | AASIRHT | AASIRHT | AASIRHT | LLIDAASIRH | AA |
| CDR2 | (SEQ ID NO: 45) | (SEQ ID NO: 45) | (SEQ ID NO: 45) | (SEQ ID NO: 46) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum6.3 Heavy chain variable region (SEQ ID NO: 68)
QVQLVQSGAEVKKPGASVKVSCKASGYSFTGYTMNWVRQAPGQGLEWIGLINPATGGTYYNQKFKDRVTLTVDTSISTAYMELSRL
RSDDTAVYYCARLGDNYRGYFASWGQGTTVTVSS
10A3D6hum6.3 Light chain variable region (SEQ ID NO: 69)
DIQLTQSPSFLSASVGDRVTITCKASQDVGTAVVWYQQKPGKAPKLLIDAASIRHTGVPSRFSGSGSGTEFTLTISSLQPEDFATYYCQQ
YSTYLYTFGGGTKLEIK

TABLE 36
Antibody 10A3D6hum6.4 Sequences

| 10A3D6hum6.4 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPATGGTYYNQ | NPATGG (SEQ ID | LINPATGGTY | WIGLINPATGGTY | INPATGGT |
| CDR2 | KFKD | NO: 38) | (SEQ ID NO: 39) | (SEQ ID NO: 40) | (SEQ ID NO: 41) |
| | (SEQ ID NO: 37) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID NO: 42) | (SEQ ID NO: 42) | (SEQ ID NO: 42) | (SEQ ID NO: 43) | (SEQ ID NO: 44) |
| Light Chain | RASQDVGTAVV | RASQDVGTAVV | RASQDVGTAVV | GTAVVWY | QDVGTA |
| CDR1 | (SEQ ID NO: 32) | (SEQ ID NO: 32) | (SEQ ID NO: 32) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum6.4 Heavy chain variable region (SEQ ID NO: 68)
QVQLVQSGAEVKKPGASVKVSCKASGYSFTGYTMNWVRQAPGQGLEWIGLINPATGGTYYNQKFKDRVTLTVDTSISTAYMELSRL
RSDDTAVYYCARLGDNYRGYFASWGQGTTVTVSS
10A3D6hum6.4 Light chain variable region (SEQ ID NO: 66)
DIQLTQSPSFLSASVGDRVTITCRASQDVGTAVVWYQQKPGKAPKLLIDWASIRHTGVPSRFSGSGSGTEFTLTISSLQPEDFATYYCQQ
YSTYLYTFGGGTKLEIK

TABLE 37
Antibody 10A3D6hum6.5 Sequences

| 10A3D6hum6.5 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD (SEQ ID | (SEQ ID NO: 10) | (SEQ ID NO: 12) | (SEQ ID NO: 14) | (SEQ ID NO: 20) |
| | NO: 4) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID NO: 42) | (SEQ ID NO: 42) | (SEQ ID NO: 42) | (SEQ ID NO: 43) | (SEQ ID NO: 44) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | AASIRHT | AASIRHT | AASIRHT | LLIDAASIRH | AA |
| CDR2 | (SEQ ID NO: 45) | (SEQ ID NO: 45) | (SEQ ID NO: 45) | (SEQ ID NO: 46) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum6.5 Heavy chain variable region (SEQ ID NO: 70)
QVQLVQSGAEVKKPGASVKVSCKASGYSFTGYTMNWVRQAPGQGLEWIGLINPNTGGTYYNQKFKDRVTLTVDTSISTAYMELSRL
RSDDTAVYYCARLGDNYRGYFASWGQGTTVTVSS
10A3D6hum6.5 Light chain variable region (SEQ ID NO: 69)
DIQLTQSPSFLSASVGDRVTITCKASQDVGTAVVWYQQKPGKAPKLLIDAASIRHTGVPSRFSGSGSGTEFTLTISSLQPEDFATYYCQQ
YSTYLYTFGGGTKLEIK

TABLE 38
Antibody 10A3D6hum6.6 Sequences

| 10A3D6hum6.6 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD | (SEQ ID NO: 10) | (SEQ ID NO: 12) | (SEQ ID NO: 14) | (SEQ ID NO: 20) |
| | (SEQ ID NO: 4) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID NO: 42) | (SEQ ID NO: 42) | (SEQ ID NO: 42) | (SEQ ID NO: 43) | (SEQ ID NO: 44) |
| Light Chain | RASQDVGTAVV | RASQDVGTAVV | RASQDVGTAVV | GTAVVWY | QDVGTA |
| CDR1 | (SEQ ID NO: 32) | (SEQ ID NO: 32) | (SEQ ID NO: 32) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum6.6 Heavy chain variable region (SEQ ID NO: 70)
QVQLVQSGAEVKKPGASVKVSCKASGYSFTGYTMNWVRQAPGQGLEWIGLINPNTGGTYYNQKFKDRVTLTVDTSISTAYMELSRL
RSDDTAVYYCARLGDNYRGYFASWGQGTTVTVSS
10A3D6hum6.6 Light chain variable region (SEQ ID NO: 66)
DIQLTQSPSFLSASVGDRVTITCRASQDVGTAVVWYQQKPGKAPKLLIDWASIRHTGVPSRFSGSGSGTEFTLTISSLQPEDFATYYCQQ
YSTYLYTFGGGTKLEIK

TABLE 39
Antibody 10A3D6hum7.1 Sequences

| 10A3D6hum7.1 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD | (SEQ ID NO: 10) | (SEQ ID NO: 12) | (SEQ ID NO: 14) | (SEQ ID NO: 20) |
| | (SEQ ID NO: 4) | | | | |
| Heavy Chain | LGDNYRGYFDA | LGDNYRGYFDA | LGDNYRGYFDA | ARLGDNYRGYFD | ARLGDNYRGYFDA |
| CDR3 | (SEQ ID NO: 31) | (SEQ ID NO: 31) | (SEQ ID NO: 31) | (SEQ ID NO: 15) | (SEQ ID NO: 32) |
| Light Chain | RASQDVGTAVV | RASQDVGTAVV | RASQDVGTAVV | GTAVVWY | QDVGTA |
| CDR1 | (SEQ ID NO: 32) | (SEQ ID NO: 32) | (SEQ ID NO: 32) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum7.1 Heavy chain variable region (SEQ ID NO: 71)
QVQLVQSGAEVKKPGASVKVSCKASGYSFTGYTMNWVRQAPGQNLEWIGLINPNTGGTYYNQKFKDRVTLTVDTSISTAYMELSRL
RSDDTAVYYCARLGDNYRGYFDAWGQGTTVTVSS
10A3D6hum7.1 Light chain variable region (SEQ ID NO: 66)
DIQLTQSPSFLSASVGDRVTITCRASQDVGTAVVWYQQKPGKAPKLLIDWASIRHTGVPSRFSGSGSGTEFTLTISSLQPEDFATYYCQQ
YSTYLYTFGGGTKLEIK

TABLE 40
Antibody 10A3D6hum7.2 Sequences

| 10A3D6hum7.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPNTGGTYYNQ | NPNTGG | LINPNTGGTY | WIGLINPNTGGTY | INPNTGGT |
| CDR2 | KFKD | (SEQ ID NO: 10) | (SEQ ID NO: 12) | (SEQ ID NO: 14) | (SEQ ID NO: 20) |
| | (SEQ ID NO: 4) | | | | |
| Heavy Chain | LGDNYRGYFDA | LGDNYRGYFDA | LGDNYRGYFDA | ARLGDNYRGYFD | ARLGDNYRGYFDA |
| CDR3 | (SEQ ID NO: 31) | (SEQ ID NO: 31) | (SEQ ID NO: 31) | (SEQ ID NO: 15) | (SEQ ID NO: 32) |
| Light Chain | RASQDVGSAVV | RASQDVGSAVV | RASQDVGSAVV | GSAVVWY | QDVGSA |
| CDR1 | (SEQ ID NO: 33) | (SEQ ID NO: 33) | (SEQ ID NO: 33) | (SEQ ID NO: 34) | (SEQ ID NO: 35) |
| Light Chain | WASIRHS | WASIRHS | WASIRHS | LLIDWASIRH | WA |
| CDR2 | (SEQ ID NO: 36) | (SEQ ID NO: 36) | (SEQ ID NO: 36) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum7.2 Heavy chain variable region (SEQ ID NO: 71)
QVQLVQSGAEVKKPGASVKVSCKASGYSFTGYTMNWVRQAPGQNLEWIGLINPNTGGTYYNQKFKDRVTLTVDTSISTAYMELSRL
RSDDTAVYYCARLGDNYRGYFDAWGQGTTVTVSS
10A3D6hum7.2 Light chain variable region (SEQ ID NO: 67)
DIQLTQSPSFLSASVGDRVTITCRASQDVGSAVVWYQQKPGKAPKLLIDWASIRHSGVPSRFSGSGSGTEFTLTISSLQPEDFATYYCQQ
YSTYLYTFGGGTKLEIK

TABLE 41
Antibody 10A3D6hum7.3 Sequences

| 10A3D6hum7.3 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPATGGTYYNQ | NPATGG | LINPATGGTY | WIGLINPATGGTY | INPATGGT |
| CDR2 | KFKD | (SEQ ID NO: 38) | (SEQ ID NO: 39) | (SEQ ID NO: 40) | (SEQ ID NO: 41) |
| | (SEQ ID NO: 37) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID NO: 42) | (SEQ ID NO: 42) | (SEQ ID NO: 42) | (SEQ ID NO: 43) | (SEQ ID NO: 44) |
| Light Chain | KASQDVGTAVV | KASQDVGTAVV | KASQDVGTAVV | GTAVVWY | QDVGTA |
| CDR1 | (SEQ ID NO: 6) | (SEQ ID NO: 6) | (SEQ ID NO: 6) | (SEQ ID NO: 16) | (SEQ ID NO: 22) |
| Light Chain | AASIRHT | AASIRHT | AASIRHT | LLIDAASIRH | AA |
| CDR2 | (SEQ ID NO: 45) | (SEQ ID NO: 45) | (SEQ ID NO: 45) | (SEQ ID NO: 46) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum7.3 Heavy chain variable region (SEQ ID NO: 72)
QVQLVQSGAEVKKPGASVKVSCKASGYSFTGYTMNWVRQAPGQNLEWIGLINPATGGTYYNQKFKDRVTLTVDTSISTAYMELSRL
RSDDTAVYYCARLGDNYRGYFASWGQGTTVTVSS
10A3D6hum7.3 Light chain variable region (SEQ ID NO: 69)
DIQLTQSPSFLSASVGDRVTITCKASQDVGTAVVWYQQKPGKAPKLLIDAASIRHTGVPSRFSGSGSGTEFTLTISSLQPEDFATYYCQQ
YSTYLYTFGGGTKLEIK

TABLE 42
Antibody 10A3D6hum7.4 Sequences

| 10A3D6hum7.4 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID NO: 3) | (SEQ ID NO: 9) | (SEQ ID NO: 11) | (SEQ ID NO: 13) | (SEQ ID NO: 19) |
| Heavy Chain | LINPATGGTYYNQ | NPATGG | LINPATGGTY | WIGLINPATGGTY | INPATGGT |
| CDR2 | KFKD | (SEQ ID NO: 38) | (SEQ ID NO: 39) | (SEQ ID NO: 40) | (SEQ ID NO: 41) |
| | (SEQ ID NO: 37) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID NO: 42) | (SEQ ID NO: 42) | (SEQ ID NO: 42) | (SEQ ID NO: 43) | (SEQ ID NO: 44) |
| Light Chain | RASQDVGTAVV | RASQDVGTAVV | RASQDVGTAVV | GTAVVWY | QDVGTA |
| CDR1 | (SEQ ID NO: 32) | (SEQ ID NO: 32) | (SEQ ID NO: 32) | (SEQ ID NO: 16) | (SEQ ID NO: 22) |
| Light Chain | WASIRHT | WASIRHT | WASIRHT | LLIDWASIRH | WA |
| CDR2 | (SEQ ID NO: 7) | (SEQ ID NO: 7) | (SEQ ID NO: 7) | (SEQ ID NO: 30) | |
| Light Chain | QQYSTYLYT | QQYSTYLYT | QQYSTYLYT | QQYSTYLY | QQYSTYLYT |
| CDR3 | (SEQ ID NO: 8) | (SEQ ID NO: 8) | (SEQ ID NO: 8) | (SEQ ID NO: 18) | (SEQ ID NO: 8) |

10A3D6hum7.4 Heavy chain variable region (SEQ ID NO: 72)
QVQLVQSGAEVKKPGASVKVSCKASGYSFTGYTMNWVRQAPGQNLEWIGLINPATGGTYYNQKFKDRVTLTVDTSISTAYMELSRL
RSDDTAVYYCARLGDNYRGYFASWGQGTTVTVSS
10A3D6hum7.4 Light chain variable region (SEQ ID NO: 66)
DIQLTQSPSFLSASVGDRVTITCRASQDVGTAVVWYQQKPGKAPKLLIDWASIRHTGVPSRFSGSGSGTEFTLTISSLOPEDFATYYCQQ
YSTYLYTFGGGTKLEIK

TABLE 43
Antibody 10A3D6hum7.5 Sequences

| 10A3D6hum7.5 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPNTGGTY | NPNTGG | LINPNTGGTY | WIGLINPNTGGTY | INPNTGGT |
| CDR2 | YNQKFKD | (SEQ ID | (SEQ ID | (SEQ ID | (SEQ ID |
| | (SEQ ID | NO: 10) | NO: 12) | NO: 14) | NO: 20) |
| | NO: 4) | | | | |
| Heavy Chain | LGDNYRGYFAS | LGDNYRGYFAS | LGDNYRGYFAS | ARLGDNYRGYFA | ARLGDNYRGYFAS |
| CDR3 | (SEQ ID | (SEQ ID | (SEQ ID | (SEQ ID | (SEQ ID |
| | NO: 42) | NO: 42) | NO: 42) | NO: 43) | NO: 44) |
| Light Chain | KASQDVGTAVV | KASQDVGTAVV | KASQDVGTAVV | GTAVVWY | QDVGTA |
| CDR1 | (SEQ ID | (SEQ ID | (SEQ ID | (SEQ ID | (SEQ ID |
| | NO: 6) | NO: 6) | NO: 6) | NO: 16) | NO: 22) |
| Light Chain | AASIRHT | AASIRHT | AASIRHT | LLIDAASIRH | AA |
| CDR2 | (SEQ ID | (SEQ ID | (SEQ ID | (SEQ ID | |
| | NO: 45) | NO: 45) | NO: 45) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum7.5 Heavy chain variable region (SEQ ID NO: 73)
QVQLVQSGAEVKKPGASVKVSCKASGYSFTGYTMNWVRQAPGQNLEWIGLINPNTGGTYYNQKFKDRVTLTVDTS
ISTAYMELSRLRSDDTAVYYCARLGDNYRGYFASWGQGTTVTVSS
10A3D6hum7.5 Light chain variable region (SEQ ID NO: 69)
DIQLTQSPSFLSASVGDRVTITCKASQDVGTAVVWYQQKPGKAPKLLIDAASIRHTGVPSRFSGSGSGTEFTLTIS
SLQPEDFATYYCQQYSTYLYTFGGGTKLEIK

TABLE 44
Antibody 10A3D6hum7.6 Sequences

| 10A3D6hum7.6 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPNTGGTYYN | NPNTGG | LINPNTGGTY | WIGLINPNTGGTY | INPNTGGT |
| CDR2 | QKFKD (SEQ | (SEQ ID | (SEQ ID | | (SEQ ID |
| | ID NO: 4) | NO: 10) | NO: 12) | | NO: 20) |
| Heavy Chain | LGDNYRGYFAS | LGDNYRGYFAS | LGDNYRGYFAS | ARLGDNYRGYFA | ARLGDNYRGYFAS |
| CDR3 | (SEQ ID | (SEQ ID | (SEQ ID | (SEQ ID | (SEQ ID |
| | NO: 42) | NO: 42) | NO: 42) | NO: 43) | NO: 44) |
| Light Chain | RASQDVGTAVV | RASQDVGTAVV | RASQDVGTAVV | GTAVVWY | QDVGTA |
| CDR1 | (SEQ ID | (SEQ ID | (SEQ ID | (SEQ ID | (SEQ ID |
| | NO: 32) | NO: 32) | NO: 32) | NO: 16) | NO: 22) |
| Light Chain | WASIRHT | WASIRHT | WASIRHT | LLIDWASIRH | WA |
| CDR2 | (SEQ ID | (SEQ ID | (SEQ ID | (SEQ ID | |
| | NO: 7) | NO: 7) | NO: 7) | NO: 30) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum7.6 Heavy chain variable region (SEQ ID NO: 73)
QVQLVQSGAEVKKPGASVKVSCKASGYSFTGYTMNWVRQAPGQNLEWIGLINPNTGGTYYNQKFKDRVTLTVDTSIS
TAYMELSRLRSDDTAVYYCARLGDNYRGYFASWGQGTTVTVSS
10A3D6hum7.6 Light chain variable region (SEQ ID NO: 66)
DIQLTQSPSFLSASVGDRVTITCRASQDVGTAVVWYQQKPGKAPKLLIDWASIRHTGVPSRFSGSGSGTEFTLTISS
LQPEDFATYYCQQYSTYLYTFGGGTKLEIK

TABLE 45
Antibody 10A3D6hum8.1 Sequences

| 10A3D6hum8.1 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPNTGGTY | NPNTGG | LINPNTGGTY | WIGLINPNTGGTY | INPNTGGT |
| CDR2 | YNQKFKD | (SEQ ID | (SEQ ID | (SEQ ID | (SEQ ID |
| | (SEQ ID | NO: 10) | NO: 12) | NO: 14) | NO: 20) |
| | NO: 4) | | | | |
| Heavy Chain | LGDNYRGYFDV | LGDNYRGYFDV | LGDNYRGYFDV | ARLGDNYRGYFD | ARLGDNYRGYFDV |
| CDR3 | (SEQ ID | (SEQ ID | (SEQ ID | (SEQ ID | (SEQ ID |
| | NO: 27) | NO: 27) | NO: 27) | NO: 15) | NO: 28) |
| Light Chain | KASQDVGTAVV | KASQDVGTAVV | KASQDVGTAVV | GTAVVWY | QDVGTA |
| CDR1 | (SEQ ID | (SEQ ID | (SEQ ID | (SEQ ID | (SEQ ID |
| | NO: 6) | NO: 6) | NO: 6) | NO: 16) | NO: 22) |
| Light Chain | WASIRHT | WASIRHT | WASIRHT | LLIDWASIRH | WA |
| CDR2 | (SEQ ID | (SEQ ID | (SEQ ID | (SEQ ID | |
| | NO: 7) | NO: 7) | NO: 7) | NO: 30) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.1 Heavy chain variable region (SEQ ID NO: 74)
QVQLVQSGAEVKKPGASVKVSCKASGYSFTGYTMNWVRQAPGQNLEWIGLINPNTGGTYYNQKFKDRVTLTVDTSIS
TAYMELSRLRSDDTAVYYCARLGDNYRGYFDVWGQGTTVTVSS
10A3D6hum8.1 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISS
LQPEDFADYFCQQYSTYLYTFGGGTKLEIK

TABLE 46
Antibody 10A3D6hum8.2 Sequences

| 10A3D6hum8.2 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGTY | NPATGG | LINPATGGTY | WIGLINPAT | INPATGGT |
| CDR2 | YNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYRGYFD | ARLGDNYRGYFDV |
| CDR3 | (SEQ ID | (SEQ ID | (SEQ ID | (SEQ ID | (SEQ ID |
| | NO: 27) | NO: 27) | NO: 27) | NO: 15) | NO: 28) |
| Light Chain | KASQDVGTAVV | KASQDVGTAVV | KASQDVGTAVV | GTAVVWY | QDVGTA |
| CDR1 | (SEQ ID | (SEQ ID | (SEQ ID | (SEQ ID | (SEQ ID |
| | NO: 6) | NO: 6) | NO: 6) | NO: 16) | NO: 22) |
| Light Chain | WASIRHT | WASIRHT | WASIRHT | LLIDWASIRH | WA |
| CDR2 | (SEQ ID | (SEQ ID | (SEQ ID | (SEQ ID | |
| | NO: 7) | NO: 7) | NO: 7) | NO: 30) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.2 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.2 Light chain variable region (SEQ ID NO: 55)
DIQLTQSPSFLSASVGDRVTITCKASQDVGTAVVWYQQKPGKAPKLLIDWASIRHTGVPSRFSGSGSGTEFTLTISS
LQPEDFADYFCQQYSTYLYTFGGGTKLEIK

TABLE 47
Antibody 10A3D6hum8.3 Sequences

| 10A3D6hum8.3 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGTY | NPATGG | LINPATGGTY | WIGLINPAT | INPATGGT |
| CDR2 | YNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYRGYFD | ARLGDNYRG |
| CDR3 | (SEQ ID | (SEQ ID | (SEQ ID | (SEQ ID | YFDV |
| | NO: 27) | NO: 27) | NO: 27) | NO: 15) | (SEQ ID |
| | | | | | NO: 28) |
| Light Chain | RASQDVGTAVV | RASQDVGTAVV | RASQDVGTAVV | GTAVVWY | QDVGTA |
| CDR1 | (SEQ ID | (SEQ ID | (SEQ ID | (SEQ ID | (SEQ ID |
| | NO: 32) | NO: 32) | NO: 32) | NO: 16) | NO: 22) |
| Light Chain | WASIRHT | WASIRHT | WASIRHT | LLIDWASIRH | WA |
| CDR2 | (SEQ ID | (SEQ ID | (SEQ ID | (SEQ ID | |
| | NO: 7) | NO: 7) | NO: 7) | NO: 30) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.3 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.3 Light chain variable region (SEQ ID NO: 78)
DIQLTQSPSFLSASVGDRVTITCRASQDVGTAVVWYQQKPGKAPKLLIDWASIRHTGVPSRFSGSGSGTEFTLTISS
LQPEDFADYFCQQYSTYLYTFGGGTKLEIK

TABLE 48
Antibody 10A3D6hum8.4 Sequences

| 10A3D6hum8.4 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGTY | NPATGG | LINPATGGTY | WIGLINPAT | INPATGGT |
| CDR2 | YNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYRGYFD | ARLGDNYRG |
| CDR3 | (SEQ ID | (SEQ ID | (SEQ ID | (SEQ ID | YFDV |
| | NO: 27) | NO: 27) | NO: 27) | NO: 15) | (SEQ ID |
| | | | | | NO: 28) |
| Light Chain | RASQDVGTAVV | RASQDVGTAVV | RASQDVGTAVV | GTAVVWY | QDVGTA |
| CDR1 | (SEQ ID | (SEQ ID | (SEQ ID | (SEQ ID | (SEQ ID |
| | NO: 32) | NO: 32) | NO: 32) | NO: 16) | NO: 22) |
| Light Chain | AASIRHT | AASIRHT | AASIRHT | LLIDAASIRH | AA |
| CDR2 | (SEQ ID | (SEQ ID | (SEQ ID | (SEQ ID | |
| | NO: 45) | NO: 45) | NO: 45) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.4 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.4 Light chain variable region (SEQ ID NO: 79)
DIQLTQSPSFLSASVGDRVTITCRASQDVGTAVVWYQQKPGKAPKLLIDAASIRHTGVPSRFSGSGSGTEFTLTISS
LQPEDFADYFCQQYSTYLYTFGGGTKLEIK

TABLE 49
Antibody 10A3D6hum8.5 Sequences

| 10A3D6hum8.5 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGT | NPATGG | LINPATGGTY | WIGLINPAT | INPATGGT |
| CDR2 | YYNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYRGYFD | ARLGDNYRG |
| CDR3 | (SEQ ID | (SEQ ID | (SEQ ID | (SEQ ID | YFDV |
| | NO: 27) | NO: 27) | NO: 27) | NO: 15) | (SEQ ID |
| | | | | | NO: 28) |
| Light Chain | RASQDVGTAVV | RASQDVGTAVV | RASQDVGTAVV | GTAVVWY | QDVGTA |
| CDR1 | (SEQ ID | (SEQ ID | (SEQ ID | (SEQ ID | (SEQ ID |
| | NO: 32) | NO: 32) | NO: 32) | NO: 16) | NO: 22) |
| Light Chain | AASIRHT | AASIRHT | AASIRHT | LLIDAASIRH | AA |
| CDR2 | (SEQ ID | (SEQ ID | (SEQ ID | (SEQ ID | |
| | NO: 45) | NO: 45) | NO: 45) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.5 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.5 Light chain variable region (SEQ ID NO: 80)
DIQLTQSPSFLSASVGDRVTITCRASQDVGTAVVWYQQKPGKAPKLLIDAASIRHTGVPSRFSGSGSGTEFTLTISS
LQPEDFATYYCQQYSTYLYTFGGGTKLEIK

TABLE 50
Antibody 10A3D6hum8.6 Sequences

| 10A3D6hum8.6 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGTYYNQ | NPATGG | LINPATGGTY | WIGLINPAT | INPATGGT |
| CDR2 | KFKD (SEQ ID | (SEQ ID | (SEQ ID | GGTY | (SEQ ID |
| | NO: 37) | NO: 38) | NO: 39) | (SEQ ID | NO: 41) |
| | | | | NO: 40) | |
| Heavy Chain | LGDNYRGYFDV | LGDNYRGYFDV | LGDNYRGYFDV | ARLGDNYRGYFD | ARLGDNYRG |
| CDR3 | (SEQ ID | (SEQ ID | (SEQ ID | (SEQ ID | YFDV |
| | NO: 27) | NO: 27) | NO: 27) | NO: 15) | (SEQ ID |
| | | | | | NO: 28) |
| Light Chain | RASQDVGSAVV | RASQDVGSAVV | RASQDVGSAVV | GSAVVWY | QDVGSA |
| CDR1 | (SEQ ID | (SEQ ID | (SEQ ID | (SEQ ID | (SEQ ID |
| | NO: 33) | NO: 33) | NO: 33) | NO: 34) | NO: 35) |
| Light Chain | AASIRHT | AASIRHT | AASIRHT | LLIDAASIRH | AA |
| CDR2 | (SEQ ID | (SEQ ID | (SEQ ID | (SEQ ID | |
| | NO: 45) | NO: 45) | NO: 45) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.6 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.6 Light chain variable region (SEQ ID NO: 81)
DIQLTQSPSFLSASVGDRVTITCRASQDVGSAVVWYQQKPGKAPKLLIDAASIRHTGVPSRFSGSGSGTEFTLTISS
LQPEDFATYYCQQYSTYLYTFGGGTKLEIK

TABLE 51
Antibody 10A3D6hum8.7 Sequences

| 10A3D6hum8.7 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGT | NPATGG | LINPATGGTY | WIGLINPAT | INPATGGT |
| CDR2 | YYNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYRGYFD | ARLGDNYRG |
| CDR3 | (SEQ ID | (SEQ ID | (SEQ ID | (SEQ ID | YFDV |
| | NO: 27) | NO: 27) | NO: 27) | NO: 15) | (SEQ ID |
| | | | | | NO: 28) |
| Light Chain | RASQDVGTAVV | RASQDVGTAVV | RASQDVGTAVV | GTAVVWY | QDVGTA |
| CDR1 | (SEQ ID | (SEQ ID | (SEQ ID | (SEQ ID | (SEQ ID |
| | NO: 32) | NO: 32) | NO: 32) | NO: 16) | NO: 22) |
| Light Chain | AASIRHS | AASIRHS | AASIRHS | LLIDAASIRH | AA |
| CDR2 | (SEQ ID | (SEQ ID | (SEQ ID | (SEQ ID | |
| | NO: 47) | NO: 47) | NO: 47) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.7 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.7 Light chain variable region (SEQ ID NO: 82)
DIQLTQSPSFLSASVGDRVTITCRASQDVGTAVVWYQQKPGKAPKLLIDAASIRHSGVPSRFSGSGSGTEFTLTISS
LQPEDFATYYCQQYSTYLYTFGGGTKLEIK

TABLE 52
Antibody 10A3D6hum8.8 Sequences

| 10A3D6hum8.8 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGT | NPATGG | LINPATGGTY | WIGLINPAT | INPATGGT |
| CDR2 | YYNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYR | ARLGDNYRG |
| CDR3 | (SEQ ID | (SEQ ID | (SEQ ID | GYFD | YFDV |
| | NO: 27) | NO: 27) | NO: 27) | (SEQ ID | (SEQ ID |
| | | | | NO: 15) | NO: 28) |
| Light Chain | RASQDVGSAVV | RASQDVGSAVV | RASQDVGSAVV | GSAVVWY | QDVGSA |
| CDR1 | (SEQ ID | (SEQ ID | (SEQ ID | (SEQ ID | (SEQ ID |
| | NO: 33) | NO: 33) | NO: 33) | NO: 34) | NO: 35) |
| Light Chain | AASIRHS | AASIRHS | AASIRHS | LLIDAASIRH | AA |
| CDR2 | (SEQ ID | (SEQ ID | (SEQ ID | (SEQ ID | |
| | NO: 47) | NO: 47) | NO: 47) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.8 Heavy chain variable region (SEQ ID NO: 75)
QVQLVQSGAEVKKPGASVKVSCKASGYSFTGYTMNWVRQAPGQNLEWIGLINPATGGTYYNQKFKDRVTLTVDTSIS
TAYMELSRLRSDDTAVYYCARLGDNYRGYFDVWGQGTTVTVSS
10A3D6hum8.8 Light chain variable region (SEQ ID NO: 83)
DIQLTQSPSFLSASVGDRVTITCRASQDVGSAVVWYQQKPGKAPKLLIDAASIRHSGVPSRFSGSGSGTEFTLTISS
LQPEDFATYYCQQYSTYLYTFGGGTKLEIK

TABLE 53
Antibody 10A3D6hum8.9 Sequences

| 10A3D6hum8.9 | Kabat | Chothia | AbM | Contact | IMGT |
|---|---|---|---|---|---|
| Heavy Chain | GYTMN | GYSFTGY | GYSFTGYTMN | TGYTMN | GYSFTGYT |
| CDR1 | (SEQ ID | (SEQ ID | (SEQ ID | (SEQ ID | (SEQ ID |
| | NO: 3) | NO: 9) | NO: 11) | NO: 13) | NO: 19) |
| Heavy Chain | LINPATGGT | NPATGG | LINPATGGTY | WIGLINPAT | INPATGGT |
| CDR2 | YYNQKFKD | (SEQ ID | (SEQ ID | GGTY | (SEQ ID |
| | (SEQ ID | NO: 38) | NO: 39) | (SEQ ID | NO: 41) |
| | NO: 37) | | | NO: 40) | |
| Heavy Chain | LGDNYRGYFDV | LGDNYRGYFDV | LGDNYRGYFDV | ARLGDNYRGYFD | ARLGDNYRG |
| CDR3 | (SEQ ID | (SEQ ID | (SEQ ID | (SEQ ID | YFDV |
| | NO: 27) | NO: 27) | NO: 27) | NO: 15) | (SEQ ID |
| | | | | | NO: 28) |
| Light Chain | RASQDVGSAVV | RASQDVGSAVV | RASQDVGSAVV | GSAVVWY | QDVGSA |
| CDR1 | (SEQ ID | (SEQ ID | (SEQ ID | (SEQ ID | (SEQ ID |
| | NO: 33) | NO: 33) | NO: 33) | NO: 34) | NO: 35) |
| Light Chain | AASIRHS | AASIRHS | AASIRHS | LLIDAASIRH | AA |
| CDR2 | (SEQ ID | (SEQ ID | (SEQ ID | (SEQ ID | |
| | NO: 47) | NO: 47) | NO: 47) | NO: 46) | |
| Light Chain | QQYSTYLYT | QQYSTYLYT | QQYSTYLYT | QQYSTYLY | QQYSTYLYT |
| CDR3 | (SEQ ID | (SEQ ID | (SEQ ID | (SEQ ID | (SEQ ID |
| | NO: 8) | NO: 8) | NO: 8) | NO: 18) | NO: 8) |

10A3D6hum8.9 Heavy chain variable region (SEQ ID NO: 84)
QVQLVQSGAEVKKPGASVKVSCKASGYSFTGYTMNWVRQAPGQGLEWIGLINPATGGTYYNQKFKDRVTLTVDTSIS
TAYMELSRLRSDDTAVYYCARLGDNYRGYFDVWGQGTTVTVSS
10A3D6hum8.9 Light chain variable region (SEQ ID NO: 83)
DIQLTQSPSFLSASVGDRVTITCRASQDVGSAVVWYQQKPGKAPKLLIDAASIRHSGVPSRFSGSGSGTEFTLTISS
LQPEDFATYYCQQYSTYLYTFGGGTKLEIK

In some embodiments, a KLRB1-binding agent comprises a heavy chain CDR1, CDR2, and CDR3 and/or a light chain CDR1, CDR2, CDR3 from an antibody described herein. In some embodiments, a KLRB1-binding agent comprises a humanized version or humanized variant of an antibody described herein. In some embodiments, a KLRB1-binding agent comprises a heavy chain CDR1, CDR2, and CDR3 and/or a light chain CDR1, CDR2, CDR3 from antibody 10A3D6 (Table 1) or a humanized version thereof. In some embodiments, a KLRB1-binding agent comprises a heavy chain CDR1, CDR2, and CDR3 and/or a light chain CDR1, CDR2, CDR3 from antibody 10A3D6C109S (Table 2) or a humanized version thereof. In some embodiments, a KLRB1-binding agent comprises a heavy chain CDR1, CDR2, and CDR3 and/or a light chain CDR1, CDR2, CDR3 from antibody 10A3D6C109F (Table 3) or a humanized version thereof. In some embodiments, a KLRB1-binding agent comprises a heavy chain CDR1, CDR2, and CDR3 and/or a light chain CDR1, CDR2, CDR3 from antibody 10A3D6C109V (Table 4) or a humanized version thereof. In some embodiments, a KLRB1-binding agent comprises a heavy chain CDR1, CDR2, and CDR3 and a light chain CDR1, CDR2, CDR3 from antibody 10A3D6hum1.2 (Table 30) or variants thereof. In some embodiments, a KLRB1-binding agent comprises a heavy chain CDR1, CDR2, and CDR3 and/or a light chain CDR1, CDR2, CDR3 from antibody 10A3D6hum1.5 (Table 31) or variants thereof. In some embodiments, a KLRB1-binding agent comprises a heavy chain CDR1, CDR2, and CDR3 and a light chain CDR1, CDR2, CDR3 from antibody 10A3D6hum2.2 (Table 32) or variants thereof. In some embodiments, a KLRB1-binding agent comprises a heavy chain CDR1, CDR2, and CDR3 and a light chain CDR1, CDR2, CDR3 from antibody 10A3D6hum6.1 (Table 33) or variants thereof. In some embodiments, a KLRB1-binding agent comprises a heavy chain CDR1, CDR2, and CDR3 and a light chain CDR1, CDR2, CDR3 from antibody 10A3D6hum6.2 (Table 34) or variants thereof. In some embodiments, a KLRB1-binding agent comprises a heavy chain CDR1, CDR2, and CDR3 and a light chain CDR1, CDR2, CDR3 from antibody 10A3D6hum6.3 (Table 35) or variants thereof. In some embodiments, a KLRB1-binding agent comprises a heavy chain CDR1, CDR2, and CDR3 and a light chain CDR1, CDR2, CDR3 from antibody 10A3D6hum6.4 (Table 36) or variants thereof. In some embodiments, a KLRB1-binding agent comprises a heavy chain CDR1, CDR2, and CDR3 and a light chain CDR1, CDR2, CDR3 from antibody 10A3D6hum6.5 (Table 37) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 38) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 39) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 40) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 41) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 42) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 43) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 44) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 45) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 46) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 47) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 48) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 49) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 50) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 51) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 52) or variants thereof. In some embodiments, a KLRB1-binding agent comprises a heavy chain CDR1, CDR2, and CDR3 and a light chain CDR1, CDR2, CDR3 from antibody 10A3D6hum6.6 (Table 53) or variants thereof. In some embodiments, a KLRB1-binding agent comprises a heavy chain CDR1, CDR2, and CDR3 and a light chain CDR1, CDR2, CDR3 from antibody 10A3D6hum1.2, 10A3D6hum1.5, 10A3D6hum2.2, 10A3D6hum6.1, 10A3D6hum6.2, 10A3D6hum6.3, 10A3D6hum6.4, 10A3D6hum6.5, 10A3D6hum6.6, 10A3D6hum7.1, 10A3D6hum7.2, 10A3D6hum7.3, 10A3D6hum7.4, 10A3D6hum7.5, 10A3D6hum7.6, 10A3D6hum8.1, 10A3D6hum8.2, 10A3D6hum8.3, 10A3D6hum8.4, 10A3D6hum8.5, 10A3D6hum8.6, 10A3D6hum8.7, 10A3D6hum8.8, or 10A3D6hum8.9 or variants thereof.

In some embodiments, a KLRB1-binding agent comprises a humanized version or humanized variant of an antibody described herein. In some embodiments, a KLRB1-binding agent comprises a heavy chain CDR1, CDR2, and CDR3 and/or a light chain CDR1, CDR2, CDR3 shown in Tables 1-4 or a humanized version thereof, e.g., as shown in Tables 5-53 or 56-57.

CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis (bioinf.org.uk/abysis/sequence_input/key_annotation/key_annotation.cgi)) are available and known to those of skill in the art for analysis of antibody sequences and determination of CDRs.

The specific CDR sequences defined herein are generally based on Kabat definitions. However, it will be understood that reference to a heavy chain CDR or CDRs and/or a light chain CDR or CDRs of a specific antibody will encompass all CDR definitions as known to those of skill in the art, e.g., as shown in the Tables herein. In some embodiments, CDR sequences used will all be identified using the same definitions, i.e., will all be Chothia, all Kabat, all IMGT, and so on.

In some embodiments, a KLRB1-binding agent is a variant of an agent described herein. In some embodiments, a KLRB1-binding agent (e.g., an antibody) comprises (a) a heavy chain CDR1, CDR2, CDR3 from a VH sequence presented herein (e.g., in Tables 1-53 or 56-57), or a variant thereof comprising 1, 2, 3, or 4 amino acid substitutions; and/or a light chain CDR1, CDR2, and/or CDR3 from a VL sequence presented herein (e.g., in Tables 1-53 or 56-57), or a variant thereof comprising 1, 2, 3, or 4 amino acid substitutions. In some embodiments, the amino acid substitutions are conservative substitutions. In some embodiments, a CDR comprises one conservative amino acid substitution. In some embodiments, a CDR comprises two conservative amino acid substitutions. In some embodiments, a CDR comprises three conservative amino acid substitutions. In some embodiments, a CDR comprises four conservative amino acid substitutions. In some embodiments, the CDR is a heavy chain CDR1. In some embodiments, the CDR is a heavy chain CDR2. In some embodiments, the CDR is a heavy chain CDR3. In some embodiments, the CDR is a light chain CDR1. In some embodiments, the CDR is a light chain CDR2. In some embodiments, the CDR is a light chain CDR3. In some embodiments, the substitutions are made as part of a humanization process. In some embodiments, the substitutions are made as part of a germline humanization process. In some embodiments, the substitutions are made as part of an affinity maturation process. In some embodiments, the substitutions are made as part of an optimization process.

In some embodiments, a KLRB1-binding agent (e.g., an antibody) comprises one or more heavy chain or light chain CDRs that have been modified, e.g., to reduce deamidation within the CDR sequence, to remove Asn (N)-glycosylation sites, to remove cysteines, or to remove Asp to reduce isomerization sites, to remove Met/Trp or Lys, e.g., to reduce the likelihood within a CDR sequence of asparagine (N)-glycosylation, cysteinylation, asparagine (Asn) deamidation, aspartate (Asp) isomerization, methionine/tryptophan (Met/Trp) oxidation, and non-enzymatic lysine (Lys) glycation (see, e.g., Haberger et al., MAbs. 2014 Mar. 1; 6 (2): 327-339; Lu et al., MAbs. 2019 January; 11 (1): 45-57). Deamidation is a chemical reaction in which an amide functional group in the side chain of the amino acids asparagine (N) or glutamine (Q) is removed or converted to another functional group. Generally, asparagine is converted to aspartic acid or isoaspartic acid and glutamine is converted to glutamic acid or polyglutamic acid. In some situations, deamidation may change the structure, function, and/or stability of a polypeptide, potentially resulting in decreased biological activity.

In certain embodiments, a KLRB1-binding agent comprises a heavy chain variable region comprising heavy chain CDRs 1, 2, and 3, and a light chain variable region comprising light chain CDRs 1, 2, and 3 as shown in Tables 1-53 or 56-57.

In some embodiments, a KLRB1-binding agent (e.g., an antibody) comprises a heavy chain variable region or sequence having at least about 80% (e.g., at least 90%, 95%, 97%, 99%, or 100%) sequence identity to a heavy chain variable region sequence presented herein (e.g., in Tables 1-53 or 56-57), and/or a light chain variable region having at least 80% (e.g., at least 90%, 95%, 97%, 99%, or 100%) sequence identity to a light chain variable region sequence presented herein (e.g., in Tables 1-53 or 56-57).

The term “consensus sequence,” as used herein with respect to light chain (VL) and heavy chain (VH) variable regions, refers to a composite or genericized VL or VH sequence defined based on information as to which amino acid residues within the VL or VH chain are amenable to modification without detriment to antigen binding. Thus, in a “consensus sequence” for a VL or VH chain, certain amino acid positions are occupied by one of multiple possible amino acid residues at that position. For example, if an arginine (R) or a serine(S) occur at a particular position, then that particular position within the consensus sequence can be either arginine or serine (R or S). Consensus sequences for VH and VL chain can be defined, for example, by in vitro affinity maturation (e.g., randomizing every amino acid position in a certain

CDR using degenerate coding primers), by scanning mutagenesis (e.g., alanine scanning mutagenesis) of amino acid residues within the antibody CDRs, or any other methods known in the art, followed by evaluation of the binding of the mutants to the antigen to determine whether the mutated amino acid position affects antigen binding. In some aspects, mutations are introduced in the CDR regions. In other aspects, mutations are introduced in framework regions. In some other aspects, mutations are introduced in CDR and framework regions. Consensus sequences can be determined by software such as the EMBOSS Cons available at: ebi.ac.uk/Tools/msa/emboss_cons/.

In some embodiments, a KLRB1-binding agent described herein (e.g., an antibody) comprises one or more constant heavy domains (e.g., CH1, CH2 and/or CH3 regions). In some embodiments, the KLRB1-binding agent comprises a constant heavy domain 1 (CH1) having an amino acid sequence set forth herein, e.g., in Tables 57 or 58. In some embodiments, the KLRB1-binding agent comprises a constant heavy domain 2 (CH2) comprising an amino acid sequence set forth herein, e.g., in Tables 57 or 58. In some embodiments, the KLRB1-binding agent comprises a constant heavy domain 3 (CH3) comprising an amino acid sequence set forth herein, e.g., in Tables 57 or 58. In some embodiments, the KLRB1-binding agent comprises a heavy chain constant region comprising an amino acid sequence having at least 80, 85%, 90%, 95%, 97%, or 99% sequence identity to an amino acid sequence set forth herein, e.g., in Tables 57 or 58. In some embodiments, the one or more constant regions of the KLRB1-binding agent has/have been modified. In some embodiments, the antibodies may comprise modifications to one or more of the three heavy chain constant regions (CH1, CH2 or CH3) and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), cell-mediated cytotoxicity (ADCC), antibody-dependent cellular phagocytosis (ADCP), complement-dependent cytotoxicity (CDC), release of inflammatory mediators, placental transfer, and control of immunoglobulin production. In some embodiments, an antibody comprises a variant Fc region. The amino acid sequences of the Fc region of human IgG1, IgG2, IgG3, and IgG4 are known to those of ordinary skill in the art (e.g., a representative human IgG1 Fc region is shown in Lobner et al., Immunol Rev. 2016 March; 270 (1): 113-131; see, e.g., Table 58). In some cases, Fc regions with amino acid variations have been identified in native antibodies. In some embodiments, a variant Fc region is engineered with substitutions at specific amino acid positions as compared to a native Fc region. In some embodiments, the Fc region is mutated to alter (reduce) antibody dependent cell-mediated cytotoxicity (ADCC), antibody induced complement dependent cytotoxicity (CDC), and/or antibody dependent cell-mediated phagocytosis (ADCP) (see, e.g., Kang and Jung, Experimental & Molecular Medicine. 2019. 51:1-9; Wang et al., Antibody Therapeutics, January 2021. 4 (1): 45-54; Lobner et al., Immunol Rev. 2016 March; 270 (1): 113-131). In some embodiments, the Fc region is afucosylated (see, e.g., Yamane-Ohnuki and Satoh, MAbs. 2009 May-Jun; 1 (3): 230-236, which describes methods for production of therapeutic antibodies with controlled levels of fucosylation of Fc region N-glycans).

In some embodiments, the modified antibodies (e.g., modified Fc region) provide for altered effector functions that, in turn, affect the biological profile of the antibody. For example, in some embodiments, mutations of a constant region enhances Fc receptor binding of the modified antibody as it circulates. In some embodiments, the constant region modifications increase the serum half-life of the antibody. In some embodiments, the constant region modifications reduce the serum half-life of the antibody. In some embodiments, the constant region modifications increase or enhance ADCC and/or complement dependent cytotoxicity (CDC) of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

Modifications to the constant region of antibodies described herein may be made using well known biochemical or molecular engineering techniques. In some embodiments, antibody variants are prepared by introducing appropriate nucleotide changes into the encoding DNA, and/or by synthesis of the desired antibody or polypeptide. Using these antibody variants it may be possible to enhance the activity or effector function provided by a specific sequence or region while substantially maintaining the structure, binding activity, and other desired characteristics of the modified antibody.

The present disclosure further embraces additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant are determined by systematically making insertions, deletions, or substitutions in the sequence and testing the resulting variant proteins for activity as compared to the parental antibody.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein, i.e., a fusion protein. In some embodiments, a variant is engineered to be detectable and may comprise a detectable label and/or protein (e.g., an enzyme).

In some embodiments, a cysteine residue not involved in maintaining the proper conformation of an antibody may be substituted or deleted to modulate the antibody's characteristics, for example, to improve oxidative stability and/or prevent aberrant disulfide crosslinking. Conversely, in some embodiments, one or more cysteine residues may be added to create disulfide bond(s) to improve stability.

The variant antibodies or polypeptides described herein may be generated using methods known in the art, including but not limited to, site-directed mutagenesis, alanine scanning mutagenesis, and PCR mutagenesis.

In some embodiments, KLRB1-binding agents described herein are chemically modified. In some embodiments, the KLRB1-binding agents are anti-KLRB1 antibodies that have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques.

The present disclosure encompasses KLRB1-binding agents built upon non-immunoglobulin backbones, wherein the agents bind to the same epitope or essentially the same epitope as an anti-KLRB1 antibody disclosed herein. In some embodiments, a non-immunoglobulin-based binding agent is an agent that competes with an anti-KLRB1 antibody described herein in a competitive binding assay. In some embodiments, alternative KLRB1-binding agents comprise a scaffold protein. Generally, scaffold proteins can be assigned to one of three groups based on the architecture of their backbone (1) scaffolds consisting of a-helices; (2) small scaffolds with few secondary structures or an irregular architecture of a-helices and β-sheets; and (3) scaffolds consisting of predominantly β-sheets. Scaffold proteins include, but are not limited to, anticalins, which are based upon the lipocalin scaffold; adnectins, which are based on the 10th domain of human fibronectin type 3; affibodies, which are based on the B-domain in the Ig-binding region of Staphylococcus aureus protein A; darpins, which are based on ankyrin repeat domain proteins; fynomers, which are based on the SH3 domain of the human Fyn protein kinase; affitins, which are based on Sac7d from Sulfolobus acidocaldarius; affilins, which are based on human y-B-crystallin or human ubiquitin; avimers, which are based on the A-domains of membrane receptor proteins; knottins (cysteine knot miniproteins), which are based upon a stable 30-amino acid anti-parallel β-strand protein fold; and Kunitz domain inhibitor scaffolds, which are based upon a structure that contains three disulfide bonds and three loops. In some embodiments, a KLRB1-binding agent comprises an engineered scaffold protein comprising a heavy chain CDR1, CDR2, and CDR3 and a light chain CDR1, CDR2, and CDR3 shown in any one of Table 1 to 54, e.g., in Table 1, 31, 32, or 33. In some embodiments, these agents are conjugated to a cytotoxic agent or moiety that results in depletion of KLRB1+ cells.

In general, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are commonly used mentioned. The binding of an antibody to its antigen is a reversible process, and the affinity of the binding is typically reported as an equilibrium dissociation constant (K_{D}). K_{D} is the ratio of an antibody dissociation rate (k_{off}) (how quickly it dissociates from its antigen) to the antibody association rate (kₒₙ) (how quickly it binds to its antigen). In some embodiments, K_{D} values are determined by measuring the kₒₙ and k_{off} rates of a specific antibody/antigen interaction and then using a ratio of these values to calculate the K_{D} value. K_{D} values may be used to evaluate and rank order the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target, (ii) valency of both the antibody and antigen, and (iii) structural arrangement of the parts that interact.

In some embodiments, a KLRB1-binding agent (e.g., an antibody) binds KLRB1 (e.g., human KLRB1) with a dissociation constant (K_{D}) of about 1 μM or less, about 100 nM or less, about 40 nM or less, about 20 nM or less, about 10 nM or less, about 1 nM or less, about 0.1 nM or less, 50 pM or less, 10 pM or less, or 1 pM or less. In some embodiments, a KLRB1-binding agent binds KLRB1 (e.g., human KLRB1) with a K_{D} of about 20 nM or less. In some embodiments, a KLRB1-binding agent binds KLRB1 (e.g., human KLRB1) with a K_{D} of about 10 nM or less. In some embodiments, a KLRB1-binding agent binds KLRB1 (e.g., human KLRB1) with a K_{D} of about 1 nM or less. In some embodiments, a KLRB1-binding agent binds KLRB1 (e.g., human KLRB1) with a K_{D} of about 0.5 nM or less. In some embodiments, a KLRB1-binding agent binds KLRB1 (e.g., human KLRB1) with a K_{D} of about 0.1 nM or less. In some embodiments, a KLRB1-binding agent binds KLRB1 (e.g., human KLRB1) with a K_{D} of about 50 pM or less. In some embodiments, a KLRB1-binding agent binds KLRB1 (e.g., human KLRB1) with a K_{D} of about 25 pM or less. In some embodiments, a KLRB1-binding agent binds KLRB1 (e.g., human KLRB1) with a K_{D} of about 10 pM or less. In some embodiments, a KLRB1-binding agent binds KLRB1 (e.g., human KLRB1) with a K_{D} of about 1 pM or less. In some embodiments, the dissociation constant of the binding agent (e.g., an antibody) for KLRB1 is the dissociation constant determined using a KLRB1 protein immobilized on a Biacore chip and the binding agent flowed over the chip. In some embodiments, the dissociation constant of the binding agent (e.g., an antibody) for KLRB1 is the dissociation constant determined using the binding agent captured by an anti-human IgG antibody on a Biacore chip and soluble KLRB1 flowed over the chip.

In some embodiments, a KLRB1-binding agent (e.g., an antibody) binds KLRB1 (e.g., human KLRB1) with a half maximal effective concentration (EC₅₀) of about 1 μM or less, about 100 nM or less, about 40 nM or less, about 20 nM or less, about 10 nM or less, about 1 nM or less, or about 0.1 nM or less. In some embodiments, a KLRB1-binding agent binds to human KLRB1 with an EC₅₀ of about 1 μM or less, about 100 nM or less, about 40 nM or less, about 20 nM or less, about 10 nM or less, about 1 nM or less, or about 0.1 nM or less. In some embodiments, a KLRB1-binding agent binds cyno KLRB1 and/or human KLRB1 with an EC₅₀ of about 40 nM or less, about 20 nM or less, about 10 nM or less, about 1 nM or less or about 0.1 nM or less.

Without being bound by theory, the antibodies disclosed herein demonstrate superiority in one or more the following activities compared to commercial antibodies:
increased binding potency (lower binding EC₅₀ to cell expressed KLRB1 or to soluble KLRB1 extracellular domain);
increased ADCC mediated depletion potency (lower EC₅₀ for depletion of CHO-KLRB1+ cells or lower EC₅₀ for reporter cell line ADCC assay);
increased binding affinity kinetics (lower K_{D} or lower K_{off});
increased blocking potency (lower IC₅₀ for inhibition of CLEC2D binding to cell expressed KLRB1); and
increased augmentation potency (lower EC₅₀ for augmenting (increasing) CLEC2D binding to cell expressed KLRB1).

In some embodiments, the antibodies disclosed herein have a higher production yield, lower immunogenicity (due to the presence of humanized variable regions and/or human Fc sequences), and/or improved biophysical parameters (e.g., higher melting temperature, greater freeze-thaw stability, lower isomerization, reduced or absence of deamidation, or less susceptibility to oxidation) when compared to commercial antibodies (e.g., B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7).

The KLRB1-binding agents (e.g., antibodies) described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, recombinant expression vectors are used to amplify and express DNA encoding antibodies, or fragments thereof, against human KLRB1. For example, recombinant expression vectors can be replicable DNA constructs which have synthetic or cDNA-derived DNA fragments encoding a polypeptide chain of a KLRB1-binding agent, such as an anti-KLRB1 antibody, or antigen-binding fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The KLRB1-binding agents (e.g., antibodies) of the present disclosure can be expressed from one or more vectors. For example, in some embodiments, a heavy chain polypeptide is expressed by one vector and a light chain polypeptide is expressed by a second vector. In some embodiments, a heavy chain polypeptide and a light chain polypeptide are expressed by one vector.

Suitable host cells for expression of a KLRB1-binding agent (e.g., an antibody) or a KLRB1 protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art. Thus, the present disclosure provides cells comprising the KLRB1-binding agents described herein. In some embodiments, the cells produce the KLRB1-binding agents described herein. In some embodiments, the cells produce an antibody. In some embodiments, the cells produce an antibody that binds human KLRB1. In some embodiments, the cells produce an antibody that binds cyno KLRB1. In some embodiments, the cells produce an antibody that binds human KLRB1 and cyno KLRB1. In some embodiments, the cells produce an antibody designated 10A3D6, 10A3D6hum1.2, 10A3D6hum1.5, 10A3D6hum2.2, 10A3D6hum6.1, 10A3D6hum6.2, 10A3D6hum6.3, 10A3D6hum6.4, 10A3D6hum6.5, 10A3D6hum6.6, or variants thereof. In some embodiments, the cells produce a scFv version of antibody 10A3D6, 10A3D6hum1.2, 10A3D6hum1.5, 10A3D6hum2.2, 10A3D6hum6.1, 10A3D6hum6.2, 10A3D6hum6.3, 10A3D6hum6.4, 10A3D6hum6.5, or 10A3D6hum6.6. In some embodiments, the cell is a prokaryotic cell (e.g., E. coli). In some embodiments, the cell is an eukaryotic cell. In some embodiments, the cell is a mammalian cell. In some embodiments, the cell is a hybridoma cell. Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF). In some embodiments, supernatants from expression systems which secrete recombinant protein into culture media are first concentrated using a commercially available protein concentration filter, for example, an Amicon® or Millipore Pellicon® ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reverse-phase HPLC steps employing hydrophobic RP-HPLC media, e.g., silica gel having pendant methyl or other aliphatic groups, are employed to further purify a recombinant protein. In some embodiments, hydrophobic interaction chromatography (HIC) is used to separate recombinant proteins based on their hydrophobicity. HIC is a useful separation technique for purifying proteins while maintaining biological activity due to the use of conditions and matrices that operate under less denaturing conditions than some other techniques. Some or all of the foregoing purification steps, in various combinations, can be employed to provide a homogeneous recombinant protein.

Anti-KLRB1 antibodies of the present disclosure may be analyzed for their physical/chemical properties and/or biological activities by various assays known in the art. In some embodiments, an anti-KLRB1 antibody is tested for its ability to bind KLRB1 (e.g., human KLRB1 and/or cyno KLRB1). Binding assays include, but are not limited to, SPR (e.g., Biacore), ELISA, and FACS. In some embodiments, an anti-KLRB1 antibody is tested for its ability to induce ADCC, ADCP, and/or CDC, as well as the ability of the antibody to kill KLRB1 target cells (cell depletion). Assays include, but are not limited to, ADCC cell lysis assays, e.g., that use LDH release and detection of formazan salt. In addition, antibodies may be evaluated for solubility, stability, thermostability, viscosity, expression levels, expression quality, and/or purification efficiency.

In some embodiments, purified anti-KLRB1 antibodies are characterized by assays including, but not limited to, N-terminal sequencing, amino acid analysis, high pressure liquid chromatography (HPLC), mass spectrometry, ion exchange chromatography, and papain digestion.

### Antibody Conjugates

The present disclosure also provides conjugates comprising an anti-KLRB1 antibody or antigen-binding fragment thereof described herein. In some embodiments, the antibody or antigen-binding fragment thereof is attached to a second molecule. In some embodiments, the antibody or antigen-binding fragment thereof is conjugated to a cytotoxic agent or moiety, e.g., that causes or promotes depletion of KLRB1-expressing cells to which the antibody or antigen-binding fragment binds. In some embodiments, the antibody or antigen-binding fragment thereof is conjugated to a cytotoxic agent to form an ADC (antibody-drug conjugate). In some embodiments, the cytotoxic agent is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic agent is a microtubule inhibitor including, but not limited to, auristatins, maytansinoids (e.g., DM1 and DM4), and tubulysins. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC1065. A derivative of any one of these toxins may be used as long as the derivative retains the cytotoxic activity of the parent molecule.

Conjugates comprising an anti-KLRB1 antibody or antigen-binding fragment thereof described herein can be made using any suitable method known in the art. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, an anti-KLRB1 antibody or antigen-binding fragment thereof described herein is conjugated to a detectable substance or molecule that allows the antibody to be used for diagnosis and/or detection. A detectable substance can include but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ⁹⁹mTc, ²⁰¹Ti, ¹³³Xe, ⁹⁰Y, ⁶⁹Yb, ¹⁷⁵Yb, ⁶⁵Zn; positron emitting metals; and magnetic metal ions.

An anti-KLRB1 antibody or antigen-binding fragment thereof described herein can also be conjugated to a second antibody to form an antibody heteroconjugate.

An anti-KLRB1 antibody or antigen-binding fragment thereof as described herein may be attached to a solid support. Such solid supports include, but are not limited to, glass, cellulose, polyacrylamide, nylon, polystyrene, polyvinyl chloride, or polypropylene. In some embodiments, immobilized anti-KLRB1 antibodies are used in immunoassays. In some embodiments, immobilized anti-KLRB1 antibodies are used in purification of the target antigen.

### Polynucleotides/Methods of Making Binding Agents

Also provided herein are nucleic acids encoding a polypeptide described herein and vectors, preferably expression vectors, containing the nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein that encode a KLRB1 binding agent as described herein.

The recombinant expression vectors of the invention can be designed for expression of KLRB1 binding agent proteins in prokaryotic cells. Preferably, the KLRB1 binding agents can be expressed in mammalian cells, preferably human cells. See, e.g., Frenzel et al., Front Immunol. 2013; 4:217. When used in mammalian cells, the expression vector's control functions are often provided by viral regulatory elements. For example, commonly used promoters are derived from polyoma, Adenovirus 2, cytomegalovirus and Simian Virus 40.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (i.e., express) a KLRB1 binding agent protein. Accordingly, the invention further provides methods for producing a KLRB1 binding agent protein using the host cells of the invention. In one embodiment, the method includes culturing the host cell of the invention (into which a recombinant expression vector encoding a KLRB1 binding agent protein has been introduced) in a suitable medium such that a KLRB1 binding agent protein is produced. In another embodiment, the method further includes isolating a KLRB1 binding agent protein from the medium or the host cell. In some embodiments, the Fc region is afucosylated (see, e.g., Yamane-Ohnuki and Satoh, MAbs. 2009 May-Jun; 1 (3): 230-236, which describes methods for production of therapeutic antibodies with controlled levels of fucosylation of Fc region N-glycans).

### Pharmaceutical Compositions

Also provided herein are pharmaceutical compositions comprising a KLRB1-binding agent as described herein as an active ingredient. In various embodiments, the KLRB1 binding agent is prepared as a pharmaceutical composition, for example as a pharmaceutical composition for use as a medicament. In various embodiments, the pharmaceutical composition is for use as a medicament for treating a disease as described herein, optionally an autoimmune disease, an allergic disease, a transplant rejection, or a hematologic malignancy, in a subject in need thereof. In some embodiments, the autoimmune disease is rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, hidradenitis suppurativa, Crohn's disease, ulcerative colitis, or celiac disease. In some embodiments, the allergic disease is asthma, allergic eosinophilic asthma, allergy, atopic dermatitis, nasal polyposis, eosinophilic gastrointestinal disorder, or hypereosinophilic syndrome. In some embodiments, the transplant rejection can be a rejection of a kidney, lung, heart, liver, limb, skin, or multi-organ transplant. In some embodiments, the hematological malignancy is a leukemia, e.g., T cell leukemia, NK cell leukemia, T cell lymphoma, T cell prolymphocytic leukemia (T-PLL), or large granular lymphocytic leukemia (LGLL). In some embodiments, the lymphoma is hepatosplenic T cell lymphoma (HSTCL), NK/T cell lymphomas (NKTCL), extranodal NK/T cell lymphomas (ENKL), aggressive NK cell leukemia (ANKL), mycosis fungoides, Sezary syndrome, peripheral T cell lymphoma, angioimmunoblastic T cell lymphoma (AITL), and peripheral T cell lymphoma not otherwise specified (PTCL-NOS). In some embodiments, the leukemia is aggressive NK cell leukemia or T cell prolymphocytic leukemia.

One skilled in the art can formulate the KLRB1-binding agent as a pharmaceutical composition according to known methods.

Pharmaceutical compositions can include a carrier. “Carriers” as used herein can include pharmaceutically acceptable carriers, excipients, or stabilizers that are nontoxic (or relatively non-toxic) to the cell or subject being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™

In various embodiments, the KLRB1 binding agent is comprised in an injectable formulation, for example, a subcutaneous, intravenous, intramuscular, intrathecal or intraperitoneal injection formulation. Injectable formulations can be aqueous solutions, for example in physiologically compatible buffers such as Hanks' solution, Ringer's solution, or physiological saline buffer. The injectable formulation can contain formulatory agents such as suspending, stabilizing and/or dispersing agents. Alternatively, the KLRB1 binding agent can be in a dried or powder form for constitution with a suitable vehicle, e.g., sterile pyrogen-free water, before use.

The binding agents of the present disclosure can be formulated in any suitable form for delivery to a target cell/tissue. In some embodiments, a KLRB1-binding agent can be formulated as a liposome, microparticle, microcapsule, albumin microsphere, microemulsion, nano-particle, nanocapsule, or macroemulsion. In some embodiments, the pharmaceutical formulation includes an agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, a KLRB1-binding agent is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### Treatment and Administration

The disclosure provides methods comprising administering a KLRB1 binding agent as described herein, or a pharmaceutical composition comprising a KLRB1 binding agent as described herein, to a subject in need thereof. In some embodiments, the subject is a human. In some embodiments, the methods are carried out in vivo (e.g., as opposed to ex vivo). As used herein, “treatment” refers to therapeutic treatment (treating a subject who has a disease); the methods can also be used for prophylactic or preventative measures, wherein the object is to prevent or slow down (lessen) the targeted pathologic condition or disorder, in a subject who does not have the disease. Those in need of treatment can include those already with the disorder, those prone to have the disorder, or those in whom the disorder is to be prevented (as used herein, “prevent” means to reduce the risk of developing).

In various aspects and embodiments, the disclosure provides methods for treating an autoimmune disease, an allergic disease, a transplant rejection, or a hematologic malignancy, in a subject in need thereof. In some embodiments, the autoimmune disease is rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis Crohn's disease, ulcerative colitis, or celiac disease. In some embodiments, the allergic disease is asthma, allergic eosinophilic asthma, allergy, atopic dermatitis, nasal polyposis, eosinophilic gastrointestinal disorder, or hypereosinophilic syndrome. In some embodiments, the transplant rejection can be a rejection of a kidney, lung, heart, liver, limb, skin, or multi-organ transplant. In some embodiments, the hematological malignancy is a leukemia, e.g., T cell leukemia, NK cell leukemia, T cell lymphoma, T cell prolymphocytic leukemia (T-PLL), or large granular lymphocytic leukemia (LGLL). In some embodiments, the lymphoma is hepatosplenic T cell lymphoma (HSTCL), NK/T cell lymphomas (NKTCL), extranodal NK/T cell lymphomas (ENKL), aggressive NK cell leukemia (ANKL), mycosis fungoides, Sezary syndrome, peripheral T cell lymphoma, angioimmunoblastic T cell lymphoma (AITL), and peripheral T cell lymphoma not otherwise specified (PTCL-NOS). In some embodiments, the leukemia is aggressive NK cell leukemia or T cell prolymphocytic leukemia. As described herein, KLRB1 expressing cells are implicated in the pathogenesis of these diseases; thus in some embodiments, the hematological malignancy expresses KLRB1. In accordance with the present disclosure, depletion of such KLRB1 expressing cells provides a therapeutic benefit.

In various aspects and embodiments, the disclosure provides methods for treating or preventing transplant rejection. The transplant rejection can be, for example, kidney rejection.

“Administration” and “treatment,” as it applies to an animal, human, experimental subject, cell, tissue, organ, or biological fluid, can include contacting an exogenous pharmaceutical, therapeutic agent, diagnostic agent, or composition to the animal, human, subject, cell, tissue, organ, or biological fluid. “Administration” and “treatment” include in vivo, as well as in some embodiments in vitro or ex vivo treatments.

Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom can vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

As such, in various embodiments, the term “effective amount” or therapeutically effective amount” is a concentration or amount of the KLRB1 binding agent that results in achieving a particular stated purpose, e.g., reduction in one or more symptoms of a disease described herein. An “effective amount” of a KLRB1 binding agent can be determined empirically. Furthermore, a “therapeutically effective amount” is a concentration or amount of a KLRB1 binding agent which is effective for achieving a stated therapeutic effect. This amount can also be determined empirically.

In some embodiments, treatment with the KLRB1-binding agents of the disclosure can kill at least about 20%, e.g., at least about 30%, 40%, 50%, 60%, 70%, or 80% of the KLRB1 expressing cells that are implicated in the pathogenesis of the diseases disclosed herein.

The term “subject” refers to any animal (e.g., a mammal), including, but not limited to, humans and non-human veterinary subjects including primates.

As used herein, reference to “about” or “approximately” a value or parameter includes (and describes) embodiments that are directed to that value or parameter. For example, a description referring to “about X” includes description of “X”. “About” as used herein means plus or minus 10 percent.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

In various embodiments, the KLRB1 binding agent can be administered by providing an mRNA encoding the binding agent to the subject.

The following examples are illustrative and not restrictive. Many variations of the technology will become apparent to those of skill in the art upon review of this disclosure. The scope of the technology should, therefore, be determined not with reference to the examples, but instead should be determined with reference to the appended claims along with their full scope of equivalents.

### EXAMPLES

### Example 1: Gene Expression of KLRB1 Marks a Unique Collection of Immune Cells, and is Limited to Immune Cells

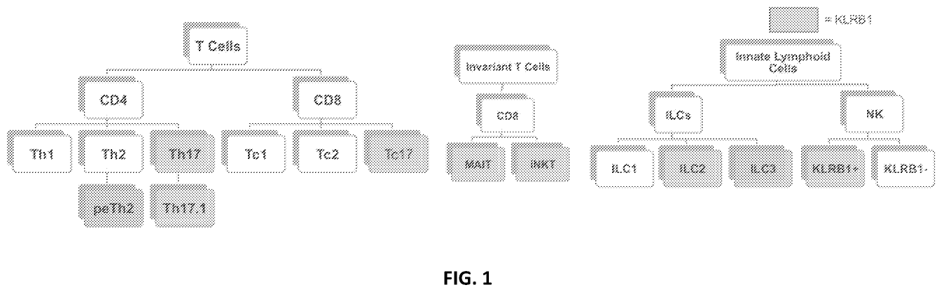

KLRB1 expression cuts across traditional lymphocyte classification and marks Th17, Th17.1, ex-Th17, Tc17, iNKTs, ILC2, ILC3, peTh2, and a subset of NK cells (FIG. 1).

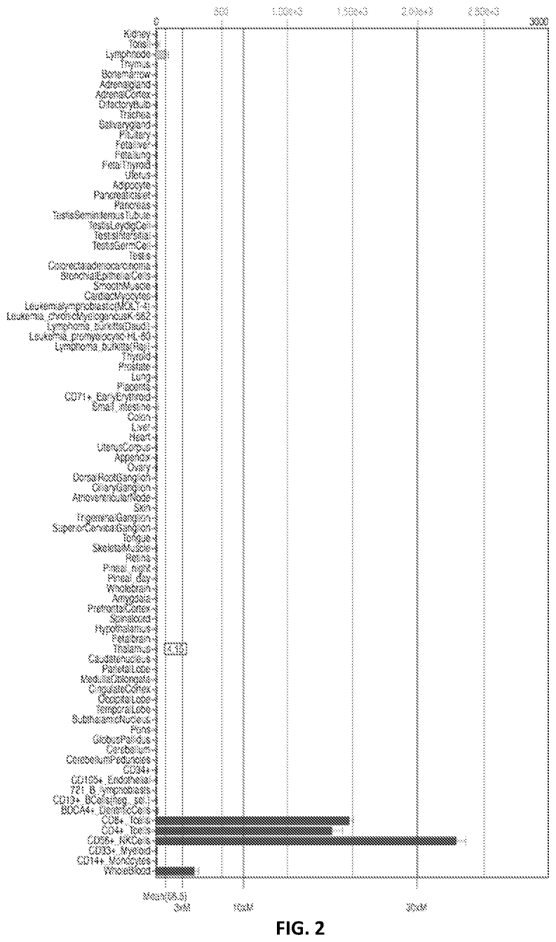

Body-wide KLRB1 expression profiling data indicates KLRB1 has no significant expression on any cell type other than immune cells (FIG. 2).

### Example 2: KLRB1 Expression is Enhanced in Various Immune Conditions

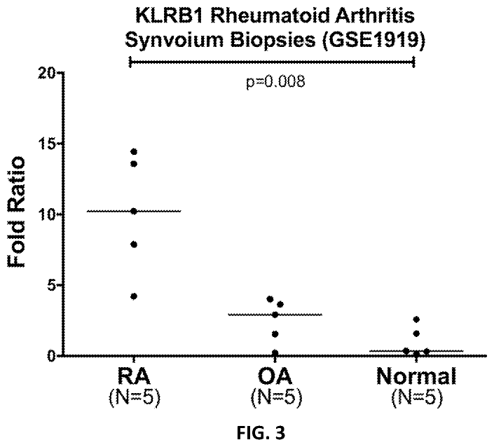

Rheumatoid arthritis: Analysis of expression data (GSE1919) from synovium biopsies from patients with rheumatoid arthritis compared to crystal-induced arthritis patients shows increased expression of KLRB1 (10 fold ratio) (FIG. 3). Accordingly, rheumatoid arthritis is a particularly attractive target for therapies according to the present disclosure.

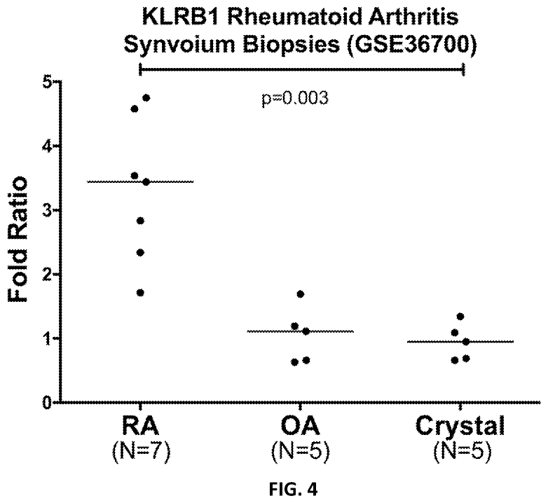

Analysis of expression data (GSE36700) from synovium biopsies from patients with rheumatoid arthritis compared to normal patients shows increased expression of KLRB1 (3.3 fold ratio) (FIG. 4). Accordingly, rheumatoid arthritis is a particularly attractive target for therapies according to the present disclosure.

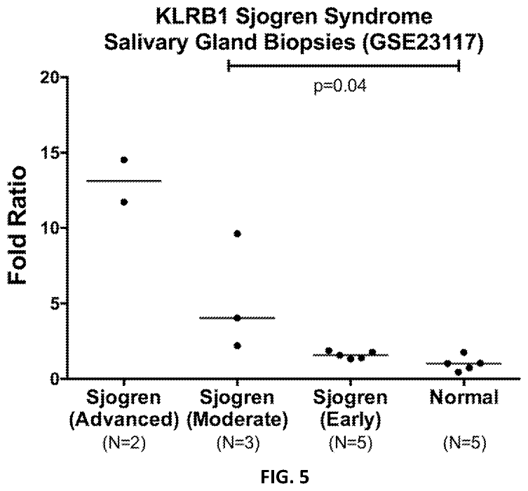

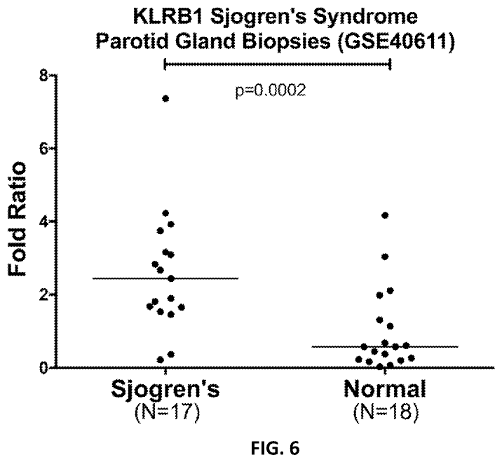

Sjögren's syndrome: Analysis of expression data (GSE23117) from salivary gland biopsies from patients with Sjogren's syndrome compared to normal shows increased expression of KLRB1 in advanced stage (13.1 fold ratio), moderate stage (5.3 fold ratio), and early stage (1.6 fold ratio) (FIG. 5). Accordingly, Sjogren's syndrome is a particularly attractive target for therapies according to the present disclosure.

Analysis of expression data (GSE40611) from parotid gland biopsies from patients with Sjogren's syndrome compared to normal shows increased expression of KLRB1 (2.6 fold ratio) (FIG. 6). Accordingly, Sjogren's syndrome is a particularly attractive target for therapies according to the present disclosure.

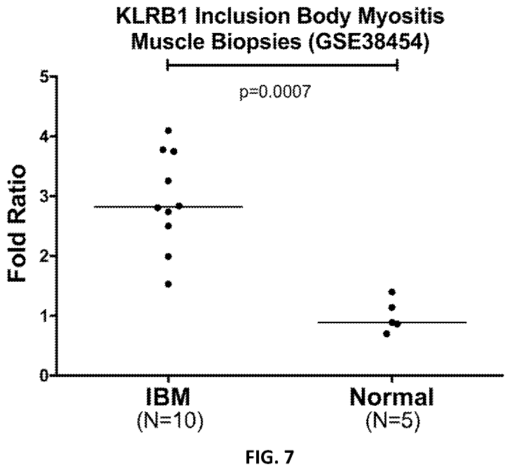

Inclusion body myositis: Analysis of expression data (GSE38454) from muscle biopsies from patients with inclusion body myositis compared to normal shows increased expression of KLRB1 (2.9 fold ratio) (FIG. 7). Accordingly, inclusion body myositis is a particularly attractive target for therapies according to the present disclosure.

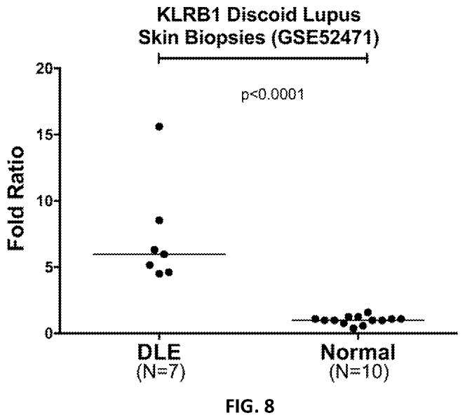

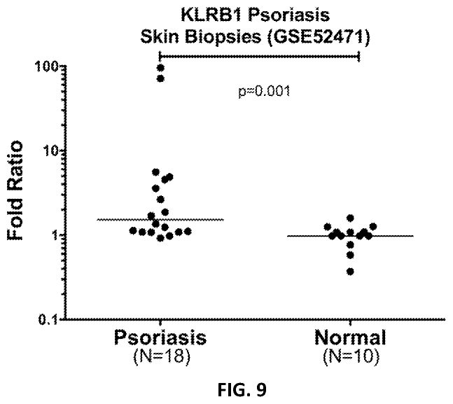

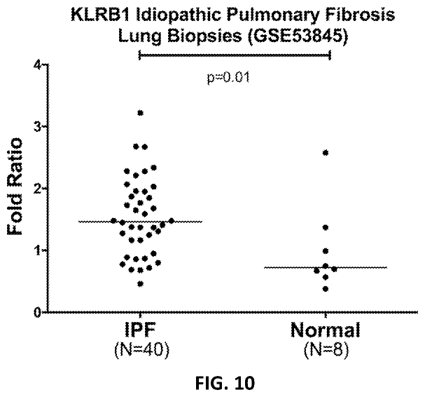

Discoid lupus: Analysis of expression data (GSE52471) from skin biopsies from patients with discoid lupus compared to normal shows increased expression of KLRB1 (7.2 fold ratio) (FIG. 8). Accordingly, discoid lupus is a particularly attractive target for therapies according to the present disclosure.

Psoriasis: Analysis of expression data (GSE52471) from skin biopsies from patients with psoriasis compared to normal shows increased expression of KLRB1 (11.2 fold ratio) (FIG. 9). Accordingly, discoid lupus is a particularly attractive target for therapies according to the present disclosure.

Idiopathic pulmonary fibrosis: Analysis of expression data (GSE53845) from lung biopsies from patients with idiopathic pulmonary fibrosis compared to normal shows increased expression of KLRB1 (1.5 fold ratio) (FIG. 10). Accordingly, idiopathic pulmonary fibrosis is a particularly attractive target for therapies according to the present disclosure.

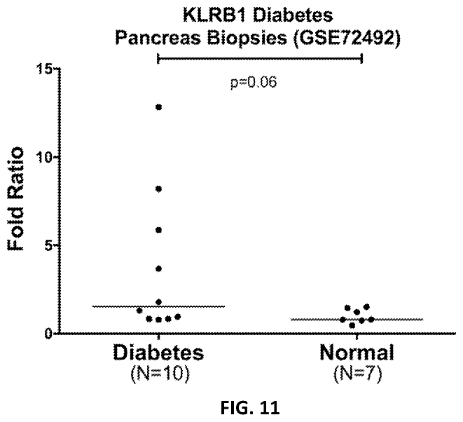

Diabetes: Analysis of expression data (GSE72492) from pancreas biopsies from patients with diabetes compared to normal shows increased expression of KLRB1 (3.7 fold ratio) (FIG. 11). Accordingly, diabetes is a particularly attractive target for therapies according to the present disclosure.

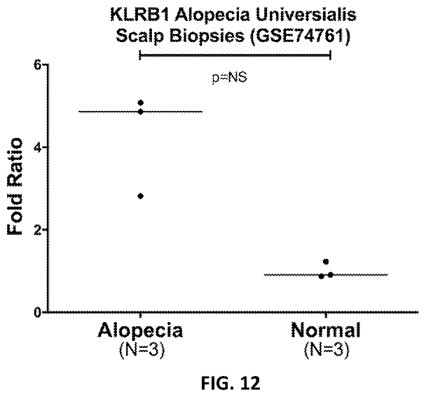

Alopecia universalis: Analysis of expression data (GSE74761) from scalp biopsies from patients with idiopathic pulmonary fibrosis compared to normal shows increased expression of KLRB1 (4.3 fold ratio) (FIG. 12). Accordingly, alopecia universalis is a particularly attractive target for therapies according to the present disclosure.

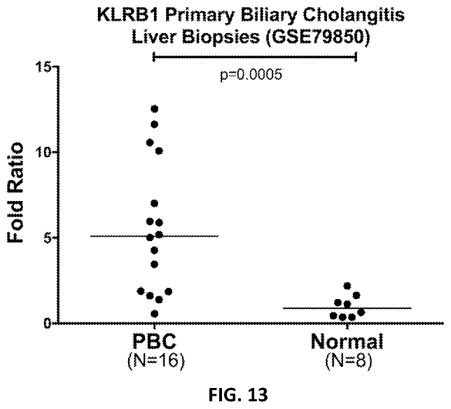

Primary biliary cholangitis: Analysis of expression data (GSE79850) from liver biopsies from patients with primary biliary cholangitis eventually requiring liver transplantation compared to normal shows increased expression of KLRB1 (5.6 fold ratio) (FIG. 13). Accordingly, primary biliary cholangitis is a particularly attractive target for therapies according to the present disclosure.

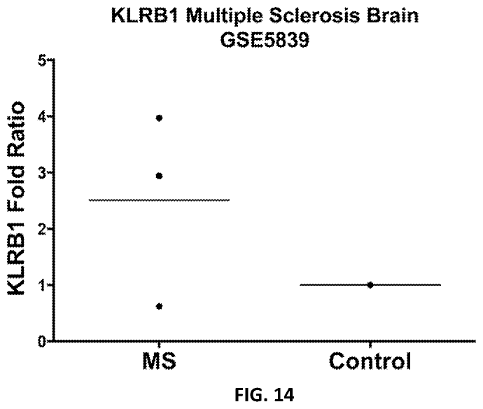

Multiple Sclerosis: Analysis of expression data (GSE5839) from brain biopsies from patients with multiple sclerosis show elevated expression of KLRB1 compared to control brain (2.5-fold) (FIG. 14). Accordingly, multiple sclerosis is a particularly attractive target for therapies according to the present disclosure.

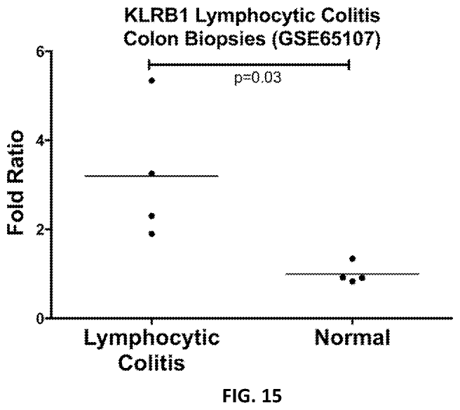

Lymphocytic colitis: Analysis of expression data (GSE65107) from colon biopsies from 4 patients with lymphocytic colitis compared to 4 healthy persons shows increased expression of KLRB1 (3.2 fold ratio) (FIG. 15). Accordingly, lymphocytic colitis is a particularly attractive target for therapies according to the present disclosure.

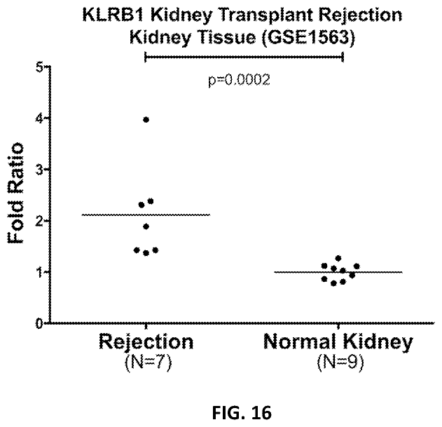

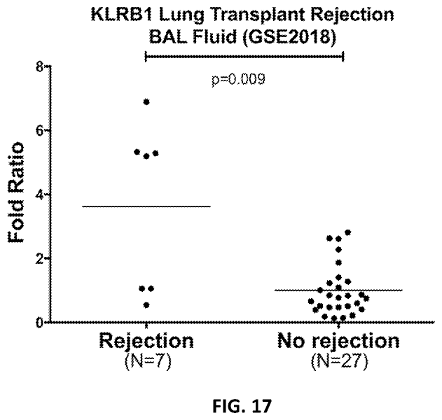

Kidney transplant rejection: Analysis of expression data (GSE1563) from kidney biopsies from 7 patients with acute kidney rejection compared to 9 healthy persons shows increased expression of KLRB1 (2.1 fold ratio) (FIG. 16). Accordingly, kidney transplant rejection is a particularly attractive target for therapies according to the present disclosure. Lung transplant: Analysis of expression data (GSE65107) from lung broncheoalveolar lavage (BAL) fluid from 7 patients with lung transplant rejection compared to 27 patients with lung transplants not undergoing rejection shows increased expression of KLRB1 (3.6 fold ratio) (FIG. 17). Accordingly, lung transplant rejection is a particularly attractive target for therapies according to the present disclosure.

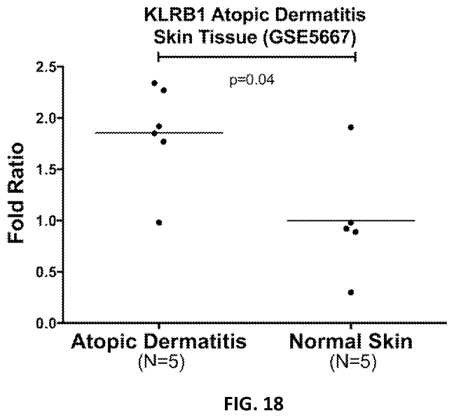

Atopic dermatitis: Analysis of expression data (GSE65107) from skin biopsies from 5 patients with atopic dermatitis compared to 5 healthy persons shows increased expression of KLRB1 (1.9 fold ratio) (FIG. 18). Accordingly, atopic dermatitis is a particularly attractive target for therapies according to the present disclosure.

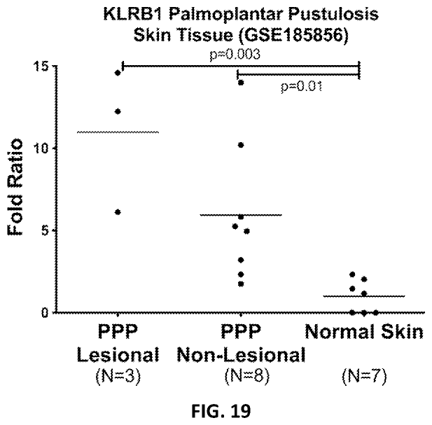

Palmoplantar pustulosis: Analysis of expression data (GSE185856) from skin biopsies from 3 patients with palmoplantar pustulosis lesional skin and 8 patients palmoplantar pustulosis non-lesional skin compared to 7 healthy persons shows increased expression of KLRB1 (11.0 fold ratio lesional to healthy; 5.9 fold ratio non-lesional to healthy) (FIG. 19). Accordingly, palmoplantar pustulosis is a particularly attractive target for therapies according to the present disclosure.

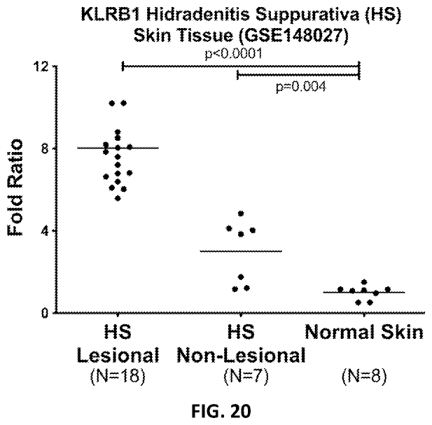

Hidradenitis suppurativa: Analysis of expression data (GSE148027) from skin biopsies from 18 patients with hidradenitis suppurativa lesional skin and 7 patients hidradenitis suppurativa non-lesional skin compared to 8 healthy persons shows increased expression of KLRB1 (8.0 fold ratio lesional to healthy; 3.0 fold ratio non-lesional to healthy) (FIG. 20). Accordingly, hidradenitis suppurativa is a particularly attractive target for therapies according to the present disclosure.

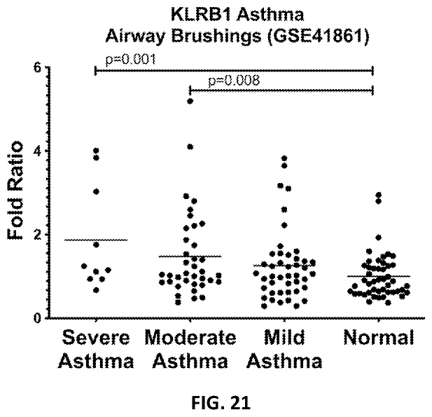

Asthma: Analysis of expression data (GSE41861) from lung airway brushings from 10 patients with severe asthma, 37 patients with moderate asthma, and 44 patients with mild asthma compared to 47 healthy persons shows increased expression of KLRB1 (1.9 fold ratio severe asthma to healthy; 1.5 fold ratio moderate asthma to healthy; and 1.3 fold ratio mild asthma to healthy) (FIG. 21). Accordingly, atopic dermatitis is a particularly attractive target for therapies according to the present disclosure.

### Example 3: KLRB1 Expression is Enhanced in Various T and NK Cell Lymphomas and Leukemias

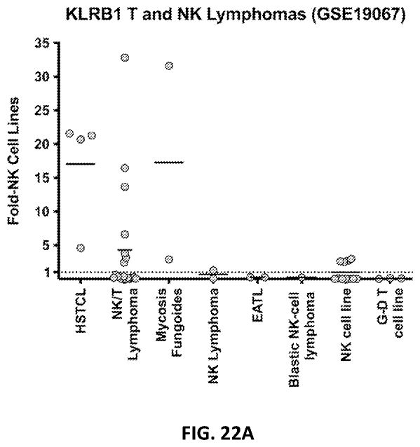

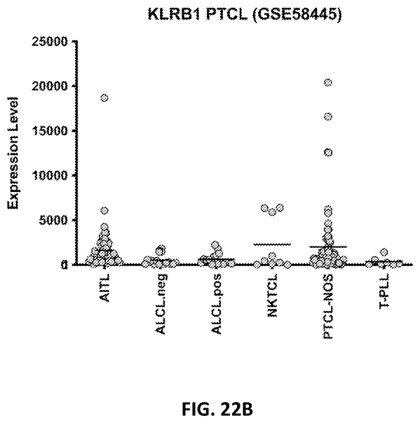

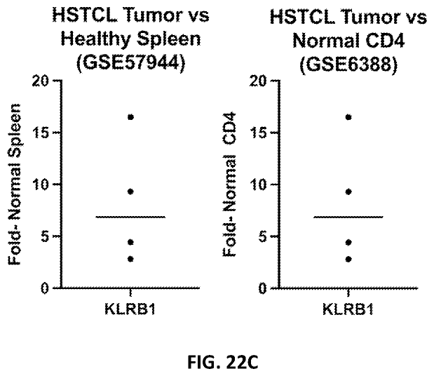

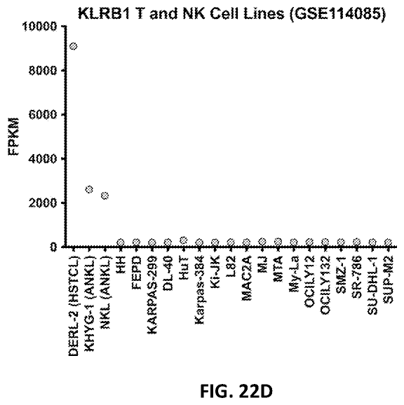

Analysis of expression data (GSE19067) from tumor cells from patients with various T and NK cell lymphomas and leukemias compared to normal NK cell expression (e.g., shows increased expression of KLRB1 (e.g., hepatosplenic T cell lymphoma has 17 fold ratio compared to normal NK cell line) (FIG. 22). Accordingly, various T and NK cell lymphomas are particularly attractive targets for therapies according to the present disclosure.

In particular, some patients with hepatosplenic T cell lymphoma (HSTCL), NK/T cell lymphomas (NKTCL), extranodal NK/T cell lymphomas (ENKL), aggressive NK cell leukemia (ANKL), mycosis fungoides, Sezary syndrome, peripheral T cell lymphoma, angioimmunoblastic T cell lymphoma (AITL), and peripheral T cell lymphoma not otherwise specified (PTCL-NOS) have increased expression of KLRB1 or similar expression of KLRB1 to CD4+ T cells (FIG. 22). Accordingly, hepatosplenic T cell lymphoma (HSTCL), NK/T cell lymphomas (NKTCL), extranodal NK/T cell lymphomas (ENKL), aggressive NK cell leukemia (ANKL), mycosis fungoides, Sezary syndrome, peripheral T cell lymphoma, angioimmunoblastic T cell lymphoma (AITL), and peripheral T cell lymphoma not otherwise specified (PTCL-NOS) are particularly attractive targets for therapies according to the present disclosure.

### Example 4: Monoclonal Anti-KLRB1 Antibody 10A3D6 Binds to Human KLRB1

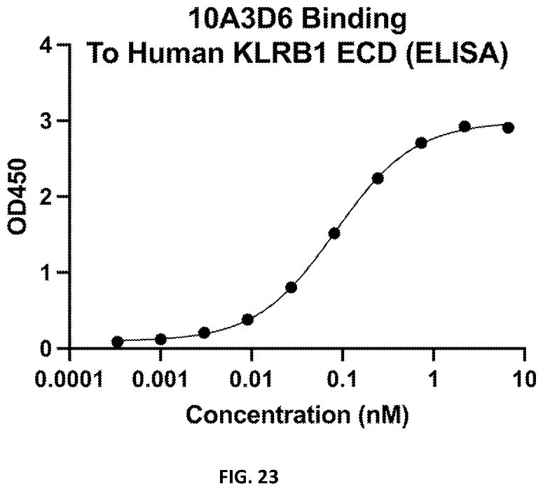

Monoclonal antibodies were generated against human KLRB1 by immunizing mice (5 balb/c and 5 SJL) with human KLRB1 protein extracellular domain (ECD) (Uniprot ID Q12918, amino acids 67-225) to create mouse hybridomas. Parental hybridoma clones were subsequently screened for binding by ELISA against human KLRB1 ECD and by FACS against stable CHO-K1 cell lines expressing human KLRB1 (SEQ ID NO: 1) and separately expressing cynomolgus KLRB1 (SEQ ID NO: 2). Twenty hybridomas were selected for subcloning based on binding to both human and cynomolgus CHO-K1 expressing KLRB1 cells, resulting in 91 subclones. These 91 subclones underwent repeat FACS binding studies to CHO-K1 human-KLRB1 and CHO-K1 cynomolgus KLRB1. One of these mAbs, designated 10A3D6, was tested for binding ability in an ELISA assay. Human KLRB1 ECD was coated on ELISA plates and the plates were incubated with 10A3D6 antibody. Secondary goat anti-mouse peroxidase labelled IgG was used to detect 10A3D6 antibodies bound to the ELISA plate (see FIG. 23).

### Example 5: Monoclonal Anti-KLRB1 Chimeric and Humanized Antibodies Bind to Human KLRB1 and Cynomolgus KLRB1

The chimeric antibody 10A3D6-hIgGlk (a mouse/human chimeric antibody with 10A3D6 variable regions and human IgG1-kappa constant regions), chimeric mutated variants (10A3D6C109S, 10A3D6C109F, and 10A3D6C109V), and humanized variants of 10A3D6 created by CDR grafting (Almagro et al. 2008) were assayed for binding against human KLRB1 extracellular domain (ECD) via surface plasma resonance (SPR). The results are shown in Table 54.

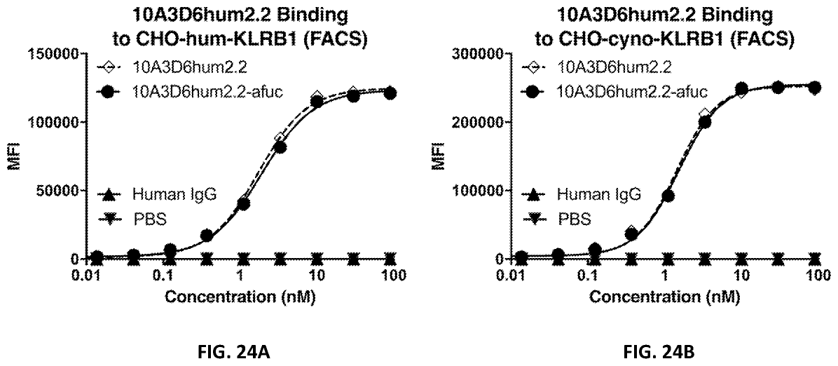

In a separate assay, humanized antibody 10A3D6hum2.2 and 10A3D6hum2.2-afuc (an afucosylated produced 10A3D6hum2.2) binding to CHO-K1 cells expressing human KLRB1 (CHO-hum-KLRB1) and CHO-K1 cells expressing cynomolgus KLRB1 (CHO-cyno-KLRB1) was assayed by FACS at a concentration range up to 90 nM. Secondary goat anti-mouse peroxidase labelled IgG was used to detect antibodies bound to CHO-hum-KLRB1 or CHO-cyno-KLRB1. 10A3D6hum2.2 and 10A3D6hum2.2-afuc had similar binding EC₅₀ (see FIG. 24).

TABLE 54
Binding kinetics by SPR for 10A3D6, 10A3D6-chimeric
variants, and 10A3D6-humanized variants

| Antibody | ka (1/Ms) | kd(1/s) | KD (M) | Rmax (RU) | Chi² (RU²) |
|---|---|---|---|---|---|
| 10A3D6 | 4.75E+05 | 1.23E−04 | 2.59E−10 | 45.2 | 0.0569 |
| 10A3D6-chimeric | 1.11E+06 | 1.91E−04 | 1.72E−10 | 57.9 | 0.0369 |
| 10A3D6C109S-chimeric | 5.93E+06 | 3.03E−04 | 5.11E−11 | 50.65 | 0.439 |
| 10A3D6C109F-chimeric | 5.36E+06 | 3.36E−04 | 6.27E−11 | 43.63 | 0.391 |
| 10A3D6C109V-chimeric | 6.72E+06 | 2.93E−04 | 4.36E−11 | 50.7 | 0.452 |
| 10A3D6C109Chum1.1 | 3.14E+05 | 2.96E−03 | 9.42E−09 | 81 | 0.00285 |
| 10A3D6C109Chum1.2 | 1.00E+06 | 9.35E−05 | 9.32E−11 | 166.6 | 0.136 |
| 10A3D6C109Chum1.3 | 9.99E+05 | 1.00E−04 | 1.00E−10 | 112.9 | 0.0621 |
| 10A3D6C109Chum1.4 | 1.20E+06 | 8.60E−05 | 7.17E−11 | 167.8 | 0.0884 |
| 10A3D6C109Chum1.5 | 1.02E+06 | 9.00E−05 | 8.81E−11 | 123.5 | 0.0486 |
| 10A3D6C109Chum2.1 | 3.15E+05 | 4.08E−03 | 1.29E−08 | 71.7 | 0.00486 |
| 10A3D6C109Chum2.2 | 9.03E+05 | 9.54E−05 | 1.06E−10 | 200 | 0.106 |
| 10A3D6C109Chum2.3 | 9.55E+05 | 9.41E−05 | 9.85E−11 | 194.6 | 0.0807 |
| 10A3D6C109Chum2.4 | 9.92E+05 | 9.68E−05 | 9.76E−11 | 194.2 | 0.116 |
| 10A3D6C109Chum2.5 | 8.61E+05 | 9.49E−05 | 1.10E−10 | 146.7 | 0.0625 |
| 10A3D6C109Chum3.1 | 4.57E+05 | 7.21E−03 | 1.58E−08 | 79.3 | 0.00531 |
| 10A3D6C109Chum3.2 | 8.32E+05 | 1.12E−04 | 1.35E−10 | 202.5 | 0.12 |
| 10A3D6C109Chum3.3 | 9.05E+05 | 9.89E−05 | 1.09E−10 | 146.2 | 0.0484 |
| 10A3D6C109Chum3.4 | 1.01E+06 | 9.20E−05 | 9.07E−11 | 153.2 | 0.0383 |
| 10A3D6C109Chum3.5 | 7.92E+05 | 1.14E−04 | 1.44E−10 | 177 | 0.0788 |
| 10A3D6C109Chum4.1 | 1.17E+05 | 2.53E−03 | 2.17E−08 | 91.9 | 0.0375 |
| 10A3D6C109Chum4.2 | 8.80E+05 | 1.07E−04 | 1.21E−10 | 176.1 | 0.0787 |
| 10A3D6C109Chum4.3 | 8.51E+05 | 1.09E−04 | 1.27E−10 | 196.6 | 0.109 |
| 10A3D6C109Chum4.4 | 9.63E+05 | 9.88E−05 | 1.03E−10 | 252.3 | 0.124 |
| 10A3D6C109Chum4.5 | 8.43E+05 | 1.07E−04 | 1.27E−10 | 125.9 | 0.0285 |
| 10A3D6C109Chum5.1 | 1.92E+06 | 1.96E−02 | 1.02E−08 | 37.4 | 0.0578 |
| 10A3D6C109Chum5.2 | 8.00E+05 | 1.38E−04 | 1.73E−10 | 221.6 | 0.116 |
| 10A3D6C109Chum5.3 | 8.68E+05 | 1.34E−04 | 1.55E−10 | 131.3 | 0.0406 |
| 10A3D6C109Chum5.4 | 9.30E+05 | 1.33E−04 | 1.43E−10 | 116 | 0.0391 |
| 10A3D6C109Chum5.5 | 8.16E+05 | 1.39E−04 | 1.70E−10 | 90.8 | 0.0175 |
| 10A3D6hum1.2 | 1.24E+06 | 1.83E−04 | 1.47E−10 | 67.6 | 0.15 |
| 10A3D6hum1.5 | 1.60E+06 | 1.78E−04 | 1.11E−10 | 59.7 | 0.66 |
| 10A3D6hum2.2 | 1.07E+06 | 1.39E−04 | 1.31E−10 | 65.9 | 0.04 |

### Example 6: Anti-KLRB1 Antibodies can Elicit Antibody-Dependent Cell-Mediated Cytotoxicity

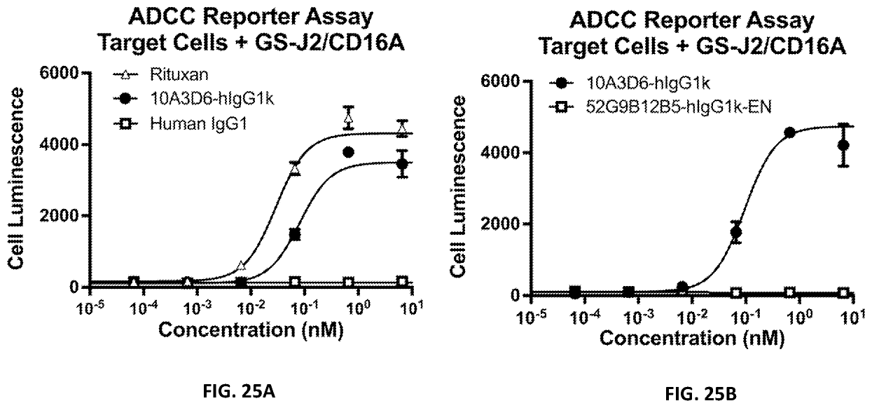

To determine whether the 10A3D6 antibodies will be effective in killing KLRB1 positive cells, the ability of these antibodies to elicit antibody-dependent cell-mediated cytotoxicity (ADCC) was tested using a reporter assay (Genscript ADCC assay with a reporter cell line GS-J2 that expresses CD16A). CHO-K1 cells expressing human KLRB1 (CHO-hum-KLRB1) were incubated with luminescent reporter cells GS-J2/CD16A and various antibodies. Rituxan (positive control) and the chimeric antibody 10A3D6-hIgGlk (a mouse/human chimeric antibody with 10A3D6 variable regions and human IgG1-kappa constant regions) show activation of luminescent reporter cells, in contrast to negative control IgG1 (FIG. 25A).

Antibody 10A3D6-hIgGlk activates luminescent reporter cells in contrast to a negative control antibody 52G9B12B5-hIgGlkEN (a mouse/human chimeric KLRB1 binding antibody with 52G9B12B5 variable regions and human IgG1-kappa mutant L234A/L235A/G237A mutation) (FIG. 25B).

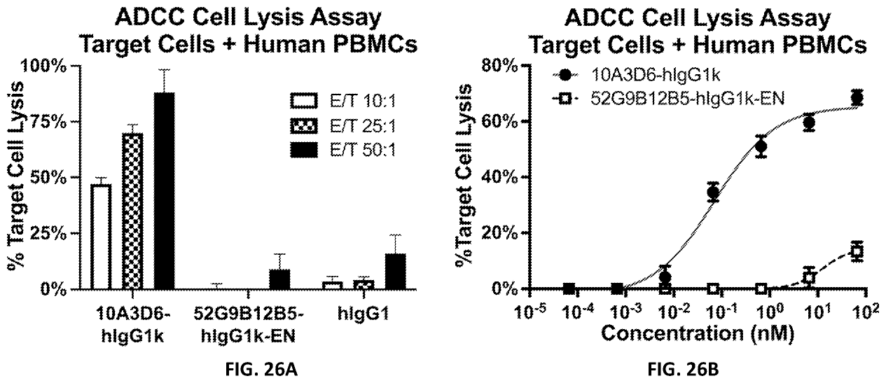

In a separate assay, ADCC elicited by the anti-KLRB1 antibody of the disclosure was evaluated using a cell lysis assay involving LDH release and detection of formazan salt (Genscript, SC1544). CHO-hum-KLRB1 were incubated with human peripheral blood mononuclear cells (PBMCs) and various antibodies. Antibody 10A3D6-hIgGlk results in target cell lysis in contrast to negative control antibody 52G9B12B5 which has an effector null mutation (FIGS. 26A-B). This confirms that the ADCC activity of anti-KLRB1 antibodies is mediated through the Fc region of the antibody.

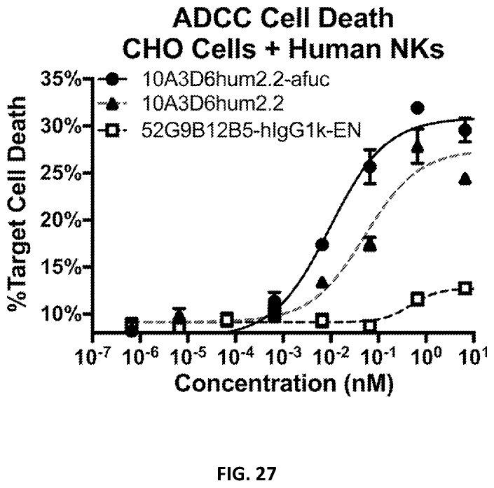

In a separate assay, an afucosylated version of 10A3D6hum2.2 (10A3D6hum2.2-afuc) was produced using FUT8 knockout cells (Satoh et al. 2006). ADCC elicited by 10A3D6hum2.2 and by afucosylated 10A3D6hum2.2 was assayed by incubating antibodies with CHO-K1 cells expressing KLRB1 (CHO-hum-KLRB1) and human NK cells and flow cytometry was used to detect target cell death. 10A3D6hum2.2-afuc had enhanced target cell killing (EC₅₀ 0.010 nM) compared with 10A3D6hum2.2 (EC₅₀ 0.052) (FIG. 27).

### Example 6: Humanized Monoclonal Anti-KLRB1 Antibodies Bind to Specific Immune Cells in Whole Blood

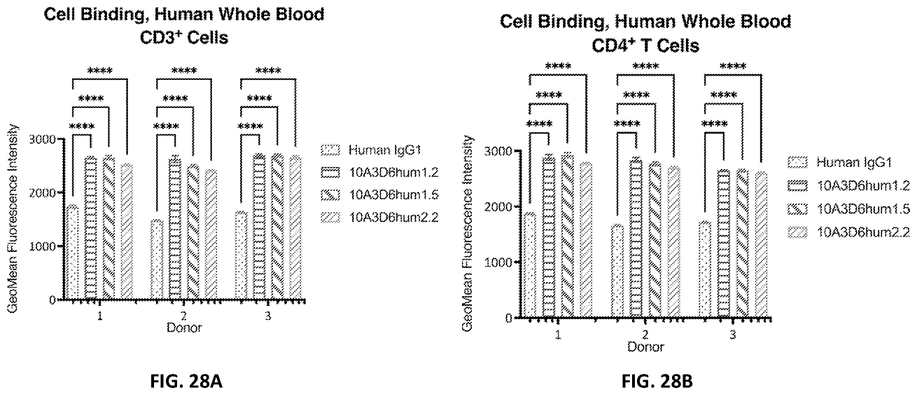

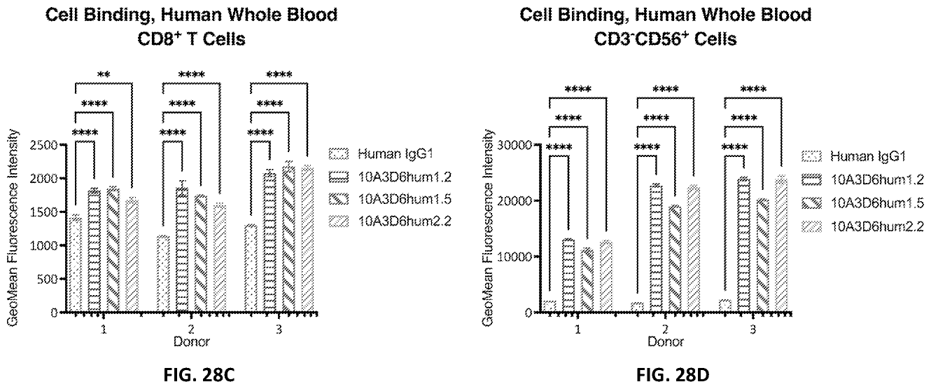

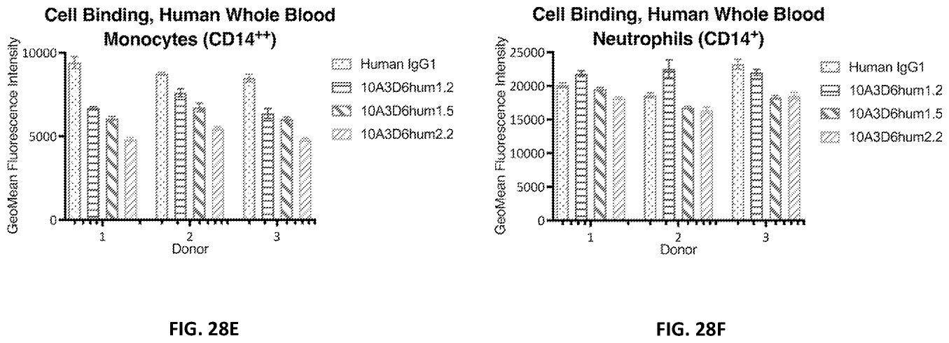

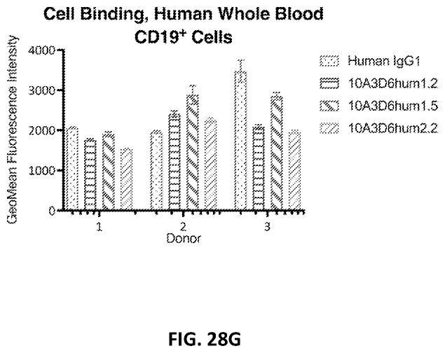

To determine whether the anti-KLRB1 antibodies of the disclosure can effectively bind to immune cells, FACS binding assays were conducted using the blood of 3 donors (experiment performed at iQ Biosciences). Fresh whole human blood was incubated with fixable viability dye, treated with TruStain Fc block (BioLegend) in FACS buffer and then incubated with 10 μg/ml of monoclonal humanized antibodies 10A3D6hum1.2, 10A3D6hum1.5, and 10A3D6hum2.2, and human IgG1 control. All three monoclonal humanized anti-KLRB1 antibodies were found to bind to CD3+ cells (FIG. 28A), CD4+ T cells (FIG. 28B), CD8+ T cells (FIG. 28C) and CD3-CD56+NK cells (FIG. 28D) at levels significantly greater than control antibody. Overall, there was no increased binding of the test antibodies to CD14++ monocytes (FIG. 28E), CD14+neutrophils (FIG. 28F), or CD19+B cells (FIG. 28G). These result show that the humanized monoclonal anti-KLRB1 antibodies of the disclosure bind to specific immune cells (in particular CD8+ and CD4+ T cells and NK cells) in whole blood.

### Example 7: Anti-KLRB1 Antibodies have Improved Binding in Competition Experiments with Commercially Available Anti-KLRB1 Antibodies

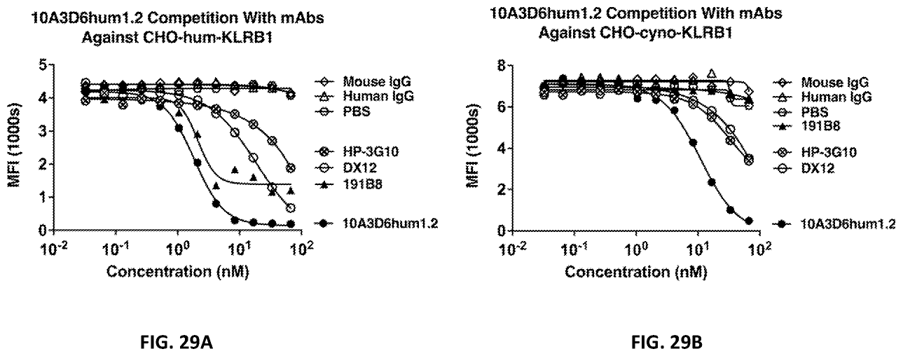

To determine whether the anti-KLRB1 antibodies of the disclosure have improved binding compared with commercially available anti-KLRB1 antibodies, competition assays were conducted. CHO-K1 cells expressing human KLRB1 (FIG. 29A) and CHO-K1 cells expressing cynomolgus KLRB1 (FIG. 29B) were incubated with fluorescently labelled antibody 10A3D6hum1.2 and then with increasing concentrations of commercial antibodies HP-3G10, DX12, and 191B8. The results show that 10A3D6 binds to a specific epitope more tightly than any of the other antibodies bind to that specific epitope.

### Example 8: Anti-KLRB1 Antibody Biophysical Characteristics

To determine the properties of the anti-KLRB1 antibodies of the disclosure, differential scanning calorimetry (DSC) was conducted. DSC measures the Tonset (onset temperature at which the melting/unfolding is first detected) and Tm (melting temperature).

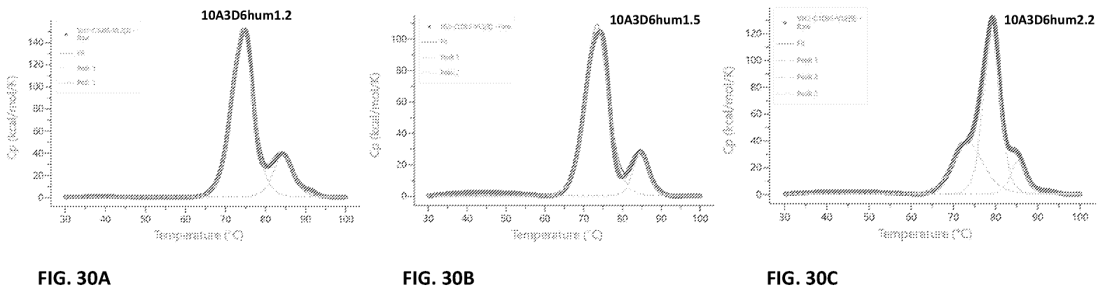

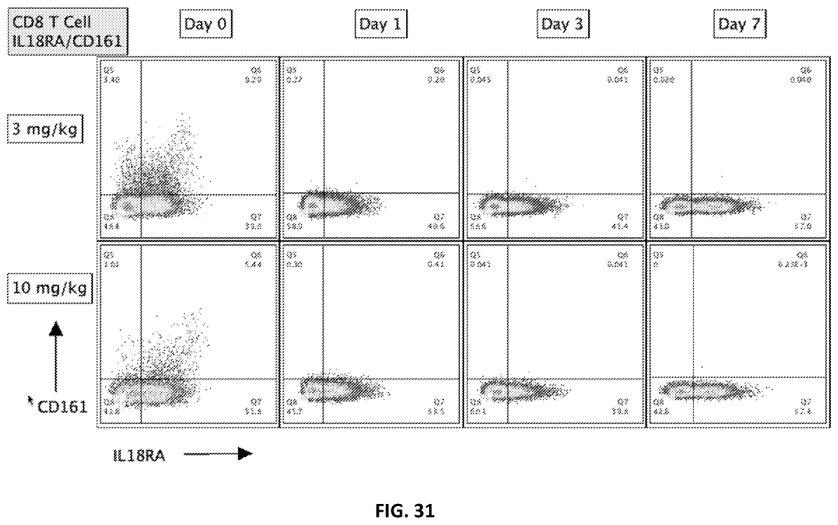

The DSC test results of the monoclonal humanized anti-KLRB1 antibodies 10A3D6hum1.2, 10A3D6hum1.5, and 10A3D6hum2.2 are shown in Table 55 below and depicted in FIGS. 30A-C.

TABLE 55
DSC test results of 10A3D6hum1.2,
10A3D6hum1.5, and 10A3D6hum2.2

| Clone ID | Tonset | Tm1 | Tm2 |
|---|---|---|---|
| 10A3D6hum1.2 | 64.96° C. | 74.10° C. | 84.31° C. |
| 10A3D6hum1.5 | 64.14° C. | 73.33° C. | 84.57° C. |
| 10A3D6hum2.2 | 65.09° C. | 73.47° C. | 78.99° C. |

TABLE 56
Sequences of exemplary antibody Variable Sequences

| | | |
|---|---|---|
| Name | Heavy Chain Variable Sequence | Light Chain Variable Sequence |

Murine

| | | |
|---|---|---|
| 10A3D6 | EVQLQQSGPELVKPGASMKISCKASGYSFTGYTMN | DIVMTQSHKFMSTSVGDRVNITCKASQDVGT |
| | WVKQSHGKNLEWIGLINPNTGGTYYNQKFKDKATL | AVVWYQQTPGQSPKVLIDWASIRHTGVPDRF |
| | TVDKSSSTAYMELLSLTSEDSAVYYCARLGDNYRG | TGSGSGTDFTLTISSVQSEDLADYFCQQYST |
| | YFDCWGQGTTLTVSS (SEQ ID NO: 48) | YLYTFGGGTKLEIK (SEQ ID NO: 49) |

Chimerics

| | | |
|---|---|---|
| 10A3D6hIgG1k | EVQLQQSGPELVKPGASMKISCKASGYSFTGYTMN | DIVMTQSHKFMSTSVGDRVNITCKASQDVGT |
| | WVKQSHGKNLEWIGLINPNTGGTYYNQKFKDKATL | AVVWYQQTPGQSPKVLIDWASIRHTGVPDRF |
| | TVDKSSSTAYMELLSLTSEDSAVYYCARLGDNYRG | TGSGSGTDFTLTISSVQSEDLADYFCQQYST |
| | YFDCWGQGTTLTVSS (SEQ ID NO: 48) | YLYTFGGGTKLEIK (SEQ ID NO: 49) |
| 10A3D6C109ShIgG1k | EVQLQQSGPELVKPGASMKISCKASGYSFTGYTMN | DIVMTQSHKFMSTSVGDRVNITCKASQDVGT |
| | WVKQSHGKNLEWIGLINPNTGGTYYNQKFKDKATL | AVVWYQQTPGQSPKVLIDWASIRHTGVPDRF |
| | TVDKSSSTAYMELLSLTSEDSAVYYCARLGDNYRG | TGSGSGTDFTLTISSVQSEDLADYFCQQYST |
| | YFDSWGQGTTLTVSS (SEQ ID NO: 50) | YLYTFGGGTKLEIK (SEQ ID NO: 49) |
| 10A3D6C109FhIgG1k | EVQLQQSGPELVKPGASMKISCKASGYSFTGYTMN | DIVMTQSHKFMSTSVGDRVNITCKASQDVGT |
| | WVKQSHGKNLEWIGLINPNTGGTYYNQKFKDKATL | AVVWYQQTPGQSPKVLIDWASIRHTGVPDRF |
| | TVDKSSSTAYMELLSLTSEDSAVYYCARLGDNYRG | TGSGSGTDFTLTISSVQSEDLADYFCQQYST |
| | YFDFWGQGTTLTVSS (SEQ ID NO: 51) | YLYTFGGGTKLEIK (SEQ ID NO: 49) |
| 10A3D6C109VhIgG1k | EVQLQQSGPELVKPGASMKISCKASGYSFTGYTMN | DIVMTQSHKFMSTSVGDRVNITCKASQDVGT |
| | WVKQSHGKNLEWIGLINPNTGGTYYNQKFKDKATL | AVVWYQQTPGQSPKVLIDWASIRHTGVPDRF |
| | TVDKSSSTAYMELLSLTSEDSAVYYCARLGDNYRG | TGSGSGTDFTLTISSVQSEDLADYFCQQYST |
| | YFDVWGQGTTLTVSS (SEQ ID NO: 52) | YLYTFGGGTKLEIK (SEQ ID NO: 49) |

Humanized

| | | |
|---|---|---|
| 10A3D6C109Chum1.1 | QVQLVQSGAEVKKPGASVKVSCKASGYSFTGYTMN | DIQLTQSPSFLSASVGDRVTITCKASQDVGT |
| | WVRQAPGQGLEWMGLINPNTGGTYYNQKFKDRVTM | AVVWYQQKPGKAPKLLIYWASIRHTGVPSRF |
| | TRDTSISTAYMELSRLRSDDTAVYYCARLGDNYRG | SGSGSGTEFTLTISSLQPEDFATYYCQQYST |
| | YFDCWGQGTTVTVSS (SEQ ID NO: 53) | YLYTFGGGTKLEIK (SEQ ID NO: 54) |
| 10A3D6C109Chum1.2 | QVQLVQSGAEVKKPGASVKVSCKASGYSFTGYTMN | DIQLTQSPSFLSASVGDRVTITCKASQDVGT |
| | WVRQAPGQGLEWMGLINPNTGGTYYNQKFKDRVTM | AVVWYQQKPGKAPKLLIDWASIRHTGVPSRF |
| | TRDTSISTAYMELSRLRSDDTAVYYCARLGDNYRG | SGSGSGTEFTLTISSLQPEDFADYFCQQYST |
| | YFDCWGQGTTVTVSS (SEQ ID NO: 53) | YLYTFGGGTKLEIK (SEQ ID NO: 55) |
| 10A3D6C109Chum1.3 | QVQLVQSGAEVKKPGASVKVSCKASGYSFTGYTMN | DIQLTQSPSFLSASVGDRVTITCKASQDVGT |
| | WVRQAPGQGLEWMGLINPNTGGTYYNQKFKDRVTM | AVVWYQQTPGKAPKLLIDWASIRHTGVPSRF |
| | TRDTSISTAYMELSRLRSDDTAVYYCARLGDNYRG | SGSGSGTEFTLTISSLQPEDFADYFCQQYST |
| | YFDCWGQGTTVTVSS (SEQ ID NO: 53) | YLYTFGGGTKLEIK (SEQ ID NO: 56) |
| 10A3D6C109Chum1.4 | QVQLVQSGAEVKKPGASVKVSCKASGYSFTGYTMN | DIQLTQSPSFLSASVGDRVTITCKASQDVGT |
| | WVRQAPGQGLEWMGLINPNTGGTYYNQKFKDRVTM | AVVWYQQTPGKAPKLLIDWASIRHTGVPDRF |
| | TRDTSISTAYMELSRLRSDDTAVYYCARLGDNYRG | SGSGSGTEFTLTISSLQPEDFADYFCQQYST |
| | YFDCWGQGTTVTVSS (SEQ ID NO: 53) | YLYTFGGGTKLEIK (SEQ ID NO: 57) |
| 10A3D6C109Chum1.5 | QVQLVQSGAEVKKPGASVKVSCKASGYSFTGYTMN | EIVMTQSPATLSVSPGERATLSCKASQDVGT |
| | WVRQAPGQGLEWMGLINPNTGGTYYNQKFKDRVTM | AVVWYQQKPGQAPRLLIDWASIRHTGIPARF |
| | TRDTSISTAYMELSRLRSDDTAVYYCARLGDNYRG | SGSGSGTEFTLTISSLOSEDFAVYYCQQYST |
| | YFDCWGQGTTVTVSS (SEQ ID NO: 53) | YLYTFGGGTKVEIK (SEQ ID NO: 58) |
| 10A3D6C109Chum2.1 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQKPGKAPKLLIYWASIRHTGVPSRF |
| | TVDTSISTAYMELSRLRSDDTAVYYCARLGDNYRG | SGSGSGTEFTLTISSLQPEDFATYYCQQYST |
| | YFDCWGQGTTVTVSS (SEQ ID NO: 59) | YLYTFGGGTKLEIK (SEQ ID NO: 54) |
| 10A3D6C109Chum2.2 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 59) | YLYTFGGGTKLEIK (SEQ ID NO: 55) |
| 10A3D6C109Chum2.3 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQTPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 59) | YLYTFGGGTKLEIK (SEQ ID NO: 56) |
| 10A3D6C109Chum2.4 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQTPGKAPKLLIDWASIRHTGVPDRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 59) | YLYTFGGGTKLEIK (SEQ ID NO: 57) |
| 10A3D6C109Chum2.5 | QVQLVQSGAEVKKPGASVKVSCKASGYSFTGYTMN | EIVMTQSPATLSVSPGERATLSCKASQDVGT |
| | WVRQAPGQNLEWIGLINPNTGGTYYNQKFKDRVTL | AVVWYQQKPGQAPRLLIDWASIRHTGIPARF |
| | TVDTSISTAYMELSRLRSDDTAVYYCARLGDNYRG | SGSGSGTEFTLTISSLQSEDFAVYYCQQYST |
| | YFDCWGQGTTVTVSS (SEQ ID NO: 59) | YLYTFGGGTKVEIK (SEQ ID NO: 58) |
| 10A3D6C109Chum3.1 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRVTL | AVVWYQQKPGKAPKLLIYWASIRHTGVPSRF |
| | TVDTSISTAYMELSRLRSDDTAVYYCARLGDNYRG | SGSGSGTEFTLTISSLQPEDFATYYCQQYST |
| | YFDCWGQGTTVTVSS (SEQ ID NO: 60) | YLYTFGGGTKLEIK (SEQ ID NO: 54) |
| 10A3D6C109Chum3.2 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 60) | YLYTFGGGTKLEIK (SEQ ID NO: 55) |
| 10A3D6C109Chum3.3 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRVTL | AVVWYQQTPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 60) | YLYTFGGGTKLEIK (SEQ ID NO: 56) |
| 10A3D6C109Chum3.4 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRVTL | AVVWYQQTPGKAPKLLIDWASIRHTGVPDRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 60) | YLYTFGGGTKLEIK (SEQ ID NO: 57) |
| 10A3D6C109Chum3.5 | QVQLVQSGAEVKKPGASVKVSCKASGYSFTGYTMN | EIVMTQSPATLSVSPGERATLSCKASQDVGT |
| | WVRQAHGQNLEWIGLINPNTGGTYYNQKFKDRVTL | AVVWYQQKPGQAPRLLIDWASIRHTGIPARF |
| | TVDTSISTAYMELSRLRSDDTAVYYCARLGDNYRG | SGSGSGTEFTLTISSLQSEDFAVYYCQQYST |
| | YFDCWGQGTTVTVSS (SEQ ID NO: 60) | YLYTFGGGTKVEIK (SEQ ID NO: 58) |
| 10A3D6C109Chum4.1 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRATL | AVVWYQQKPGKAPKLLIYWASIRHTGVPSRF |
| | TVDTSISTAYMELSRLRSDDTAVYYCARLGDNYRG | SGSGSGTEFTLTISSLQPEDFATYYCQQYST |
| | YFDCWGQGTTVTVSS (SEQ ID NO: 61) | YLYTFGGGTKLEIK (SEQ ID NO: 54) |
| 10A3D6C109Chum4.2 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRATL | AVVWYQQKPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 61) | YLYTFGGGTKLEIK (SEQ ID NO: 55) |
| 10A3D6C109Chum4.3 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRATL | AVVWYQQTPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 61) | YLYTFGGGTKLEIK (SEQ ID NO: 56) |
| 10A3D6C109Chum4.4 | QVQLVQSGAEVKKPGASVKVSCKASGYSFTGYTMN | DIQLTQSPSFLSASVGDRVTITCKASQDVGT |
| | WVRQAHGQNLEWIGLINPNTGGTYYNQKFKDRATL | AVVWYQQTPGKAPKLLIDWASIRHTGVPDRF |
| | TVDTSISTAYMELSRLRSDDTAVYYCARLGDNYRG | SGSGSGTEFTLTISSLQPEDFADYFCQQYST |
| | YFDCWGQGTTVTVSS (SEQ ID NO: 61) | YLYTFGGGTKLEIK (SEQ ID NO: 57) |
| 10A3D6C109Chum4.5 | QVQLVQSGAEVKKPGASVKVSCKASGYSFTGYTMN | EIVMTQSPATLSVSPGERATLSCKASQDVGT |
| | WVRQAHGQNLEWIGLINPNTGGTYYNQKFKDRATL | AVVWYQQKPGQAPRLLIDWASIRHTGIPARF |
| | TVDTSISTAYMELSRLRSDDTAVYYCARLGDNYRG | SGSGSGTEFTLTISSLQSEDFAVYYCQQYST |
| | YFDCWGQGTTVTVSS (SEQ ID NO: 61) | YLYTFGGGTKVEIK (SEQ ID NO: 58) |
| 10A3D6C109Chum5.1 | QVQLVQSGAEVKKPGASVKVSCKASGYSFTGYTMN | DIQLTQSPSFLSASVGDRVTITCKASQDVGT |
| | WVRQAPGQNLEWMGLINPNTGGTYYNQKFKDRVTL | AVVWYQQKPGKAPKLLIYWASIRHTGVPSRF |
| | TVDTSISTAYMELSRLRSDDTVVYYCARLGDNYRG | SGSGSGTEFTLTISSLQPEDFATYYCQQYST |
| | YFDCWGQGTLVTVSS (SEQ ID NO: 62) | YLYTFGGGTKLEIK (SEQ ID NO: 54) |
| 10A3D6C109Chum5.2 | QVQLVQSGAEVKKPGASVKVSCKASGYSFTGYTMN | DIQLTQSPSFLSASVGDRVTITCKASQDVGT |
| | WVRQAPGQNLEWMGLINPNTGGTYYNQKFKDRVTL | AVVWYQQKPGKAPKLLIDWASIRHTGVPSRF |
| | TVDTSISTAYMELSRLRSDDTVVYYCARLGDNYRG | SGSGSGTEFTLTISSLQPEDFADYFCQQYST |
| | YFDCWGQGTLVTVSS (SEQ ID NO: 62) | YLYTFGGGTKLEIK (SEQ ID NO: 55) |
| 10A3D6C109Chum5.3 | QVQLVQSGAEVKKPGASVKVSCKASGYSFTGYTMN | DIQLTQSPSFLSASVGDRVTITCKASQDVGT |
| | WVRQAPGQNLEWMGLINPNTGGTYYNQKFKDRVTL | AVVWYQQTPGKAPKLLIDWASIRHTGVPSRF |
| | TVDTSISTAYMELSRLRSDDTVVYYCARLGDNYRG | SGSGSGTEFTLTISSLQPEDFADYFCQQYST |
| | YFDCWGQGTLVTVSS (SEQ ID NO: 62) | YLYTFGGGTKLEIK (SEQ ID NO: 56) |
| 10A3D6C109Chum5.4 | QVQLVQSGAEVKKPGASVKVSCKASGYSFTGYTMN | DIQLTQSPSFLSASVGDRVTITCKASQDVGT |
| | WVRQAPGQNLEWMGLINPNTGGTYYNQKFKDRVTL | AVVWYQQTPGKAPKLLIDWASIRHTGVPDRF |
| | TVDTSISTAYMELSRLRSDDTVVYYCARLGDNYRG | SGSGSGTEFTLTISSLQPEDFADYFCQQYST |
| | YFDCWGQGTLVTVSS (SEQ ID NO: 62) | YLYTFGGGTKLEIK (SEQ ID NO: 57) |
| 10A3D6C109Chum5.5 | QVQLVQSGAEVKKPGASVKVSCKASGYSFTGYTMN | EIVMTQSPATLSVSPGERATLSCKASQDVGT |
| | WVRQAPGQNLEWMGLINPNTGGTYYNQKFKDRVTL | AVVWYQQKPGQAPRLLIDWASIRHTGIPARF |
| | TVDTSISTAYMELSRLRSDDTVVYYCARLGDNYRG | SGSGSGTEFTLTISSLQSEDFAVYYCQQYST |
| | YFDCWGQGTLVTVSS (SEQ ID NO: 62) | YLYTFGGGTKVEIK (SEQ ID NO: 58) |
| 10A3D6hum1.2 | QVQLVQSGAEVKKPGASVKVSCKASGYSFTGYTMN | DIQLTQSPSFLSASVGDRVTITCKASQDVGT |
| | WVRQAPGQGLEWMGLINPNTGGTYYNQKFKDRVTM | AVVWYQQKPGKAPKLLIDWASIRHTGVPSRF |
| | TRDTSISTAYMELSRLRSDDTAVYYCARLGDNYRG | SGSGSGTEFTLTISSLQPEDFADYFCQQYST |
| | YFDSWGQGTTVTVSS (SEQ ID NO: 63) | YLYTFGGGTKLEIK (SEQ ID NO: 55) |
| 10A3D6hum1.5 | QVQLVQSGAEVKKPGASVKVSCKASGYSFTGYTMN | EIVMTQSPATLSVSPGERATLSCKASQDVGT |
| | WVRQAPGQGLEWMGLINPNTGGTYYNQKFKDRVTM | AVVWYQQKPGQAPRLLIDWASIRHTGIPARF |
| | TRDTSISTAYMELSRLRSDDTAVYYCARLGDNYRG | SGSGSGTEFTLTISSLQSEDFAVYYCQQYST |
| | YFDSWGQGTTVTVSS (SEQ ID NO: 63) | YLYTFGGGTKVEIK (SEQ ID NO: 58) |
| 10A3D6hum2.2 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDSWGQGTTVTVSS (SEQ ID NO: 64) | YLYTFGGGTKLEIK (SEQ ID NO: 55) |
| 10A3D6hum6.1 | QVQLVQSGAEVKKPGASVKVSCKASGYSFTGYTMN | DIQLTQSPSFLSASVGDRVTITCRASQDVGT |
| | WVRQAPGQGLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFATYYCQQYST |
| | YFDAWGQGTTVTVSS (SEQ ID NO: 65) | YLYTFGGGTKLEIK (SEQ ID NO: 66) |
| 10A3D6hum6.2 | QVQLVQSGAEVKKPGASVKVSCKASGYSFTGYTMN | DIQLTQSPSFLSASVGDRVTITCRASQDVGS |
| | WVRQAPGQGLEWIGLINPNTGGTYYNQKFKDRVTL | AVVWYQQKPGKAPKLLIDWASIRHSGVPSRF |
| | TVDTSISTAYMELSRLRSDDTAVYYCARLGDNYRG | SGSGSGTEFTLTISSLQPEDFATYYCQQYST |
| | YFDAWGQGTTVTVSS (SEQ ID NO: 65) | YLYTFGGGTKLEIK (SEQ ID NO: 67) |
| 10A3D6hum6.3 | QVQLVQSGAEVKKPGASVKVSCKASGYSFTGYTMN | DIQLTQSPSFLSASVGDRVTITCKASQDVGT |
| | WVRQAPGQGLEWIGLINPATGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 68) | YLYTFGGGTKLEIK (SEQ ID NO: 69) |
| 10A3D6hum6.4 | QVQLVQSGAEVKKPGASVKVSCKASGYSFTGYTMN | DIQLTQSPSFLSASVGDRVTITCRASQDVGT |
| | WVRQAPGQGLEWIGLINPATGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 68) | YLYTFGGGTKLEIK (SEQ ID NO: 66) |
| 10A3D6hum6.5 | QVQLVQSGAEVKKPGASVKVSCKASGYSFTGYTMN | DIQLTQSPSFLSASVGDRVTITCKASQDVGT |
| | WVRQAPGQGLEWIGLINPNTGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 70) | YLYTFGGGTKLEIK (SEQ ID NO: 69) |
| 10A3D6hum6.6 | QVQLVQSGAEVKKPGASVKVSCKASGYSFTGYTMN | DIQLTQSPSFLSASVGDRVTITCRASQDVGT |
| | WVRQAPGQGLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 70) | YLYTFGGGTKLEIK (SEQ ID NO: 66) |
| 10A3D6hum7.1 | QVQLVQSGAEVKKPGASVKVSCKASGYSFTGYTMN | DIQLTQSPSFLSASVGDRVTITCRASQDVGT |
| | WVRQAPGQNLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFATYYCQQYST |
| | YFDAWGQGTTVTVSS (SEQ ID NO: 71) | YLYTFGGGTKLEIK (SEQ ID NO: 66) |
| 10A3D6hum7.2 | QVQLVQSGAEVKKPGASVKVSCKASGYSFTGYTMN | DIQLTQSPSFLSASVGDRVTITCRASQDVGS |
| | WVRQAPGQNLEWIGLINPNTGGTYYNQKFKDRVTL | AVVWYQQKPGKAPKLLIDWASIRHSGVPSRF |
| | TVDTSISTAYMELSRLRSDDTAVYYCARLGDNYRG | SGSGSGTEFTLTISSLQPEDFATYYCQQYST |
| | YFDAWGQGTTVTVSS (SEQ ID NO: 71) | YLYTFGGGTKLEIK (SEQ ID NO: 67) |
| 10A3D6hum7.3 | QVQLVQSGAEVKKPGASVKVSCKASGYSFTGYTMN | DIQLTQSPSFLSASVGDRVTITCKASQDVGT |
| | WVRQAPGQNLEWIGLINPATGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 72) | YLYTFGGGTKLEIK (SEQ ID NO: 69) |
| 10A3D6hum7.4 | QVQLVQSGAEVKKPGASVKVSCKASGYSFTGYTMN | DIQLTQSPSFLSASVGDRVTITCRASQDVGT |
| | WVRQAPGQNLEWIGLINPATGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 72) | YLYTFGGGTKLEIK (SEQ ID NO: 66) |
| 10A3D6hum7.5 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 73) | YLYTFGGGTKLEIK (SEQ ID NO: 69) |
| 10A3D6hum7.6 | QVQLVQSGAEVKKPGASVKVSCKASGYSFTGYTMN | DIQLTQSPSFLSASVGDRVTITCRASQDVGT |
| | WVRQAPGQNLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFATYYCQQYST |
| | YFASWGQGTTVTVSS (SEQ ID NO: 73) | YLYTFGGGTKLEIK (SEQ ID NO: 66) |
| 10A3D6hum8.1 | QVQLVQSGAEVKKPGASVKVSCKASGYSFTGYTMN | DIQLTQSPSFLSASVGDRVTITCKASQDVGT |
| | WVRQAPGQNLEWIGLINPNTGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDVWGQGTTVTVSS (SEQ ID NO: 74) | YLYTFGGGTKLEIK (SEQ ID NO: 55) |
| 10A3D6hum8.2 | QVQLVQSGAEVKKPGASVKVSCKASGYSFTGYTMN | DIQLTQSPSFLSASVGDRVTITCKASQDVGT |
| | WVRQAPGQNLEWIGLINPATGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLOPEDFADYFCQQYST |
| | YFDVWGQGTTVTVSS (SEQ ID NO: 75) | YLYTFGGGTKLEIK (SEQ ID NO: 55) |
| 10A3D6hum8.3 | QVQLVQSGAEVKKPGASVKVSCKASGYSFTGYTMN | DIQLTQSPSFLSASVGDRVTITCRASQDVGT |
| | WVRQAPGQNLEWIGLINPATGGTYYNQKFKDRVTL | AVVWYQQKPGKAPKLLIDWASIRHTGVPSRF |
| | TVDTSISTAYMELSRLRSDDTAVYYCARLGDNYRG | SGSGSGTEFTLTISSLQPEDFADYFCQQYST |
| | YFDVWGQGTTVTVSS (SEQ ID NO: 75) | YLYTFGGGTKLEIK (SEQ ID NO: 78) |
| 10A3D6hum8.4 | QVQLVQSGAEVKKPGASVKVSCKASGYSFTGYTMN | DIQLTQSPSFLSASVGDRVTITCRASQDVGT |
| | WVRQAPGQNLEWIGLINPATGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFADYFCQQYST |
| | YFDVWGQGTTVTVSS (SEQ ID NO: 75) | YLYTFGGGTKLEIK (SEQ ID NO: 79) |
| 10A3D6hum8.5 | QVQLVQSGAEVKKPGASVKVSCKASGYSFTGYTMN | DIQLTQSPSFLSASVGDRVTITCRASQDVGT |
| | WVRQAPGQNLEWIGLINPATGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFATYYCQQYST |
| | YFDVWGQGTTVTVSS (SEQ ID NO: 75) | YLYTFGGGTKLEIK (SEQ ID NO: 80) |
| 10A3D6hum8.6 | QVQLVQSGAEVKKPGASVKVSCKASGYSFTGYTMN | DIQLTQSPSFLSASVGDRVTITCRASQDVGS |
| | WVRQAPGQNLEWIGLINPATGGTYYNQKFKDRVTL | AVVWYQQKPGKAPKLLIDAASIRHTGVPSRF |
| | TVDTSISTAYMELSRLRSDDTAVYYCARLGDNYRG | SGSGSGTEFTLTISSLQPEDFATYYCQQYST |
| | YFDVWGQGTTVTVSS (SEQ ID NO: 75) | YLYTFGGGTKLEIK (SEQ ID NO: 81) |
| 10A3D6hum8.7 | QVQLVQSGAEVKKPGASVKVSCKASGYSFTGYTMN | DIQLTQSPSFLSASVGDRVTITCRASQDVGT |
| | WVRQAPGQNLEWIGLINPATGGTYYNQKFKDRVTL | AVVWYQQKPGKAPKLLIDAASIRHSGVPSRF |
| | TVDTSISTAYMELSRLRSDDTAVYYCARLGDNYRG | SGSGSGTEFTLTISSLQPEDFATYYCQQYST |
| | YFDVWGQGTTVTVSS (SEQ ID NO: 75) | YLYTFGGGTKLEIK (SEQ ID NO: 82) |
| 10A3D6hum8.8 | QVQLVQSGAEVKKPGASVKVSCKASGYSFTGYTMN | DIQLTQSPSFLSASVGDRVTITCRASQDVGS |
| | WVRQAPGQNLEWIGLINPATGGTYYNQKFKDRVTL | AVVWYQQKPGKAPKLLIDAASIRHSGVPSRF |
| | TVDTSISTAYMELSRLRSDDTAVYYCARLGDNYRG | SGSGSGTEFTLTISSLQPEDFATYYCQQYST |
| | YFDVWGQGTTVTVSS (SEQ ID NO: 75) | YLYTFGGGTKLEIK (SEQ ID NO: 83) |
| | QVQLVQSGAEVKKPGASVKVSCKASGYSFTGYTMN | DIQLTQSPSFLSASVGDRVTITCRASQDVGS |
| 10A3D6hum8.9 | WVRQAPGQGLEWIGLINPATGGTYYNQKFKDRVTL | AVVWYQQKPGKAPKLLIDAASIRHSGVPSRF |
| | TVDTSISTAYMELSRLRSDDTAVYYCARLGDNYRG | SGSGSGTEFTLTISSLQPEDFATYYCQQYST |
| | YFDVWGQGTTVTVSS (SEQ ID NO: 84) | YLYTFGGGTKLEIK (SEQ ID NO: 83) |

TABLE 57
Sequences of Exemplary Complete HC and LC Regions

| Ab | Complete HC | Complete LC |
|---|---|---|
| 10A3D6 | EVQLQQSGPELVKPGASMKISCKASGY | DIVMTQSHKFMSTSVGDRVNITCKASQ |
| | SFTGYTMNWVKQSHGKNLEWIGLINP | DVGTAVVWYQQTPGQSPKVLIDWASI |
| | NTGGTYYNQKFKDKATLTVDKSSSTA | RHTGVPDRFTGSGSGTDFTLTISSVQSE |
| | YMELLSLTSEDSAVYYCARLGDNYRG | DLADYFCQQYSTYLYTFGGGTKLEIKR |
| | YFDCWGQGTTLTVSSAKTTPPSVYPLA | ADAAPTVSIFPPSSEQLTSGGASVVCFL |
| | PGSAAQTNSMVTLGCLVKGYFPEPVTV | NNFYPKDINVKWKIDGSERQNGVLNS |
| | TWNSGSLSSGVHTFPAVLQSDLYTLSSS | WTDQDSKDSTYSMSSTLTLTKDEYERH |
| | VTVPSSPRPSETVTCNVAHPASSTKVD | NSYTCEATHKTSTSPIVKSFNRNEC |
| | KKIVPRDCGCKPCICTVPEVSSVFIFPPK | (SEQ ID NO: 93) |
| | PKDVLTITLTPKVTCVVVDISKDDPEVQ | |
| | FSWFVDDVEVHTAQTQPREEQFNSTFR | |
| | SVSELPIMHQDWLNGKEFKCRVNSAAF | |
| | PAPIEKTISKTKGRPKAPQVYTIPPPKEQ | |
| | MAKDKVSLTCMITDFFPEDITVEWQW | |
| | NGQPAENYKNTQPIMNINGSYFVYSKL | |
| | NVQKSNWEAGNTFTCSVLHEGLHNHH | |
| | TEKSLSHSPGK (SEQ ID NO: 92) | |
| 10A3D6hIgG1k | EVQLQQSGPELVKPGASMKISCKASGY | DIVMTQSHKFMSTSVGDRVNITCKASQ |
| | SFTGYTMNWVKQSHGKNLEWIGLINP | DVGTAVVWYQQTPGQSPKVLIDWASI |
| | NTGGTYYNQKFKDKATLTVDKSSSTA | RHTGVPDRFTGSGSGTDFTLTISSVQSE |
| | YMELLSLTSEDSAVYYCARLGDNYRG | DLADYFCQQYSTYLYTFGGGTKLEIKR |
| | YFDCWGQGTTLTVSSASTKGPSVFPLA | TVAAPSVFIFPPSDEQLKSGTASVVCLL |
| | PSSKSTSGGTAALGCLVKDYFPEPVTVS | NNFYPREAKVQWKVDNALQSGNSQES |
| | WNSGALTSGVHTFPAVLQSSGLYSLSS | VTEQDSKDSTYSLSSTLTLSKADYEKH |
| | VVTVPSSSLGTQTYICNVNHKPSNTKV | KVYACEVTHQGLSSPVTKSFNRGEC |
| | DKKVEPKSCDKTHTCPPCPAPELLGGP | (SEQ ID NO: 95) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 94) | |
| 10A3D6hum1.1 | QVQLVQSGAEVKKPGASVKVSCKASG | DIQLTQSPSFLSASVGDRVTITCKASQD |
| | YSFTGYTMNWVRQAPGQGLEWMGLI | VGTAVVWYQQKPGKAPKLLIYWASIR |
| | NPNTGGTYYNQKFKDRVTMTRDTSIST | HTGVPSRFSGSGSGTEFTLTISSLQPEDF |
| | AYMELSRLRSDDTAVYYCARLGDNYR | ATYYCQQYSTYLYTFGGGTKLEIKRTV |
| | GYFDCWGQGTTVTVSSASTKGPSVFPL | AAPSVFIFPPSDEQLKSGTASVVCLLNN |
| | APSSKSTSGGTAALGCLVKDYFPEPVT | FYPREAKVQWKVDNALQSGNSQESVT |
| | VSWNSGALTSGVHTFPAVLQSSGLYSL | EQDSKDSTYSLSSTLTLSKADYEKHKV |
| | SSVVTVPSSSLGTQTYICNVNHKPSNTK | YACEVTHQGLSSPVTKSFNRGEC (SEQ |
| | VDKKVEPKSCDKTHTCPPCPAPELLGG | ID NO: 97) |
| | PSVFLFPPKPKDTLMISRTPEVTCVVVD | |
| | VSHEDPEVKFNWYVDGVEVHNAKTKP | |
| | REEQYNSTYRVVSVLTVLHQDWLNGK | |
| | EYKCKVSNKALPAPIEKTISKAKGQPRE | |
| | PQVYTLPPSRDELTKNQVSLTCLVKGF | |
| | YPSDIAVEWESNGQPENNYKTTPPVLD | |
| | SDGSFFLYSKLTVDKSRWQQGNVFSCS | |
| | VMHEALHNHYTQKSLSLSPGK (SEQ | |
| | ID NO: 96) | |
| 10A3D6hum1.2 | QVQLVQSGAEVKKPGASVKVSCKASG | DIQLTQSPSFLSASVGDRVTITCKASQD |
| | YSFTGYTMNWVRQAPGQGLEWMGLI | VGTAVVWYQQKPGKAPKLLIDWASIR |
| | NPNTGGTYYNQKFKDRVTMTRDTSIST | HTGVPSRFSGSGSGTEFTLTISSLQPEDF |
| | AYMELSRLRSDDTAVYYCARLGDNYR | ADYFCQQYSTYLYTFGGGTKLEIKRTV |
| | GYFDCWGQGTTVTVSSASTKGPSVFPL | AAPSVFIFPPSDEQLKSGTASVVCLLNN |
| | APSSKSTSGGTAALGCLVKDYFPEPVT | FYPREAKVQWKVDNALQSGNSQESVT |
| | VSWNSGALTSGVHTFPAVLQSSGLYSL | EQDSKDSTYSLSSTLTLSKADYEKHKV |
| | SSVVTVPSSSLGTQTYICNVNHKPSNTK | YACEVTHQGLSSPVTKSFNRGEC (SEQ |
| | VDKKVEPKSCDKTHTCPPCPAPELLGG | ID NO: 98) |
| | PSVFLFPPKPKDTLMISRTPEVTCVVVD | |
| | VSHEDPEVKFNWYVDGVEVHNAKTKP | |
| | REEQYNSTYRVVSVLTVLHQDWLNGK | |
| | EYKCKVSNKALPAPIEKTISKAKGQPRE | |
| | PQVYTLPPSRDELTKNQVSLTCLVKGF | |
| | YPSDIAVEWESNGQPENNYKTTPPVLD | |
| | SDGSFFLYSKLTVDKSRWQQGNVFSCS | |
| | VMHEALHNHYTQKSLSLSPGK (SEQ | |
| | ID NO: 96) | |
| 10A3D6hum2.2 | QVQLVQSGAEVKKPGASVKVSCKASG | DIQLTQSPSFLSASVGDRVTITCKASQD |
| | YSFTGYTMNWVRQAPGQNLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ADYFCQQYSTYLYTFGGGTKLEIKRTV |
| | YFDSWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 98) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 99) | |
| 10A3D6hum2.2-EN | QVQLVQSGAEVKKPGASVKVSCKASG | DIQLTQSPSFLSASVGDRVTITCKASQD |
| | YSFTGYTMNWVRQAPGQNLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ADYFCQQYSTYLYTFGGGTKLEIKRTV |
| | YFDSWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPEAAGAP | ID NO: 98) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 100) | |
| 10A3D6hum6.1 | QVQLVQSGAEVKKPGASVKVSCKASG | DIQLTQSPSFLSASVGDRVTITCRASQD |
| | YSFTGYTMNWVRQAPGQGLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFDAWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 102) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 101) | |
| 10A3D6hum6.2 | QVQLVQSGAEVKKPGASVKVSCKASG | DIQLTQSPSFLSASVGDRVTITCRASQD |
| | YSFTGYTMNWVRQAPGQGLEWIGLIN | VGSAVVWYQQKPGKAPKLLIDWASIR |
| | PNTGGTYYNQKFKDRVTLTVDTSISTA | HSGVPSRFSGSGSGTEFTLTISSLQPEDF |
| | YMELSRLRSDDTAVYYCARLGDNYRG | ATYYCQQYSTYLYTFGGGTKLEIKRTV |
| | YFDAWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 103) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 101) | |
| 10A3D6hum6.3 | QVQLVQSGAEVKKPGASVKVSCKASG | DIQLTQSPSFLSASVGDRVTITCKASQD |
| | YSFTGYTMNWVRQAPGQGLEWIGLIN | VGTAVVWYQQKPGKAPKLLIDAASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 105) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 104) | |
| 10A3D6hum6.4 | QVQLVQSGAEVKKPGASVKVSCKASG | DIQLTQSPSFLSASVGDRVTITCRASQD |
| | YSFTGYTMNWVRQAPGQGLEWIGLIN | VGTAVVWYQQKPGKAPKLLIDWASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 102) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 104) | |
| 10A3D6hum6.5 | QVQLVQSGAEVKKPGASVKVSCKASG | DIQLTQSPSFLSASVGDRVTITCKASQD |
| | YSFTGYTMNWVRQAPGQGLEWIGLIN | VGTAVVWYQQKPGKAPKLLIDAASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 105) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 106) | |
| 10A3D6hum6.6 | QVQLVQSGAEVKKPGASVKVSCKASG | DIQLTQSPSFLSASVGDRVTITCRASQD |
| | YSFTGYTMNWVRQAPGQGLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 102) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 106) | |
| 10A3D6hum7.1 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFDAWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 102) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 107) | |
| 10A3D6hum7.2 | QVQLVQSGAEVKKPGASVKVSCKASG | DIQLTQSPSFLSASVGDRVTITCRASQD |
| | YSFTGYTMNWVRQAPGQNLEWIGLIN | VGSAVVWYQQKPGKAPKLLIDWASIR |
| | PNTGGTYYNQKFKDRVTLTVDTSISTA | HSGVPSRFSGSGSGTEFTLTISSLQPEDF |
| | YMELSRLRSDDTAVYYCARLGDNYRG | ATYYCQQYSTYLYTFGGGTKLEIKRTV |
| | YFDAWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 103) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 107) | |
| 10A3D6hum7.3 | QVQLVQSGAEVKKPGASVKVSCKASG | DIQLTQSPSFLSASVGDRVTITCKASQD |
| | YSFTGYTMNWVRQAPGQNLEWIGLIN | VGTAVVWYQQKPGKAPKLLIDAASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 105) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 108) | |
| 10A3D6hum7.4 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDWASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 102) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 108) | |
| 10A3D6hum7.5 | QVQLVQSGAEVKKPGASVKVSCKASG | DIQLTQSPSFLSASVGDRVTITCKASQD |
| | YSFTGYTMNWVRQAPGQNLEWIGLIN | VGTAVVWYQQKPGKAPKLLIDAASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 105) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 109) | |
| 10A3D6hum7.6 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFASWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 102) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 109) | |
| 10A3D6hum8.1 | QVQLVQSGAEVKKPGASVKVSCKASG | DIQLTQSPSFLSASVGDRVTITCKASQD |
| | YSFTGYTMNWVRQAPGQNLEWIGLIN | VGTAVVWYQQKPGKAPKLLIDWASIR |
| | PNTGGTYYNQKFKDRVTLTVDTSISTA | HTGVPSRFSGSGSGTEFTLTISSLQPEDF |
| | YMELSRLRSDDTAVYYCARLGDNYRG | ADYFCQQYSTYLYTFGGGTKLEIKRTV |
| | YFDVWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 98) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 110) | |
| 10A3D6hum8.2 | QVQLVQSGAEVKKPGASVKVSCKASG | DIQLTQSPSFLSASVGDRVTITCKASQD |
| | YSFTGYTMNWVRQAPGQNLEWIGLIN | VGTAVVWYQQKPGKAPKLLIDWASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ADYFCQQYSTYLYTFGGGTKLEIKRTV |
| | YFDVWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 98) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.3 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDWASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ADYFCQQYSTYLYTFGGGTKLEIKRTV |
| | YFDVWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC |
| | DKKVEPKSCDKTHTCPPCPAPELLGGP | (SEQ ID NO: 112) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.4 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDAASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ADYFCQQYSTYLYTFGGGTKLEIKRTV |
| | YFDVWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 113) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.5 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDAASIR |
| | PATGGTYYNQKFKDRVTLTVDTSISTA | HTGVPSRFSGSGSGTEFTLTISSLQPEDF |
| | YMELSRLRSDDTAVYYCARLGDNYRG | ATYYCQQYSTYLYTFGGGTKLEIKRTV |
| | YFDVWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 114) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.6 | QVQLVQSGAEVKKPGASVKVSCKASG | DIQLTQSPSFLSASVGDRVTITCRASQD |
| | YSFTGYTMNWVRQAPGQNLEWIGLIN | VGSAVVWYQQKPGKAPKLLIDAASIRH |
| | PATGGTYYNQKFKDRVTLTVDTSISTA | TGVPSRFSGSGSGTEFTLTISSLQPEDFA |
| | YMELSRLRSDDTAVYYCARLGDNYRG | TYYCQQYSTYLYTFGGGTKLEIKRTVA |
| | YFDVWGQGTTVTVSSASTKGPSVFPLA | APSVFIFPPSDEQLKSGTASVVCLLNNF |
| | PSSKSTSGGTAALGCLVKDYFPEPVTVS | YPREAKVQWKVDNALQSGNSQESVTE |
| | WNSGALTSGVHTFPAVLQSSGLYSLSS | QDSKDSTYSLSSTLTLSKADYEKHKVY |
| | VVTVPSSSLGTQTYICNVNHKPSNTKV | ACEVTHQGLSSPVTKSFNRGEC (SEQ |
| | DKKVEPKSCDKTHTCPPCPAPELLGGP | ID NO: 115) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.7 | QVQLVQSGAEVKKPGASVKVSCKASG | DIQLTQSPSFLSASVGDRVTITCRASQD |
| | YSFTGYTMNWVRQAPGQNLEWIGLIN | VGTAVVWYQQKPGKAPKLLIDAASIR |
| | PATGGTYYNQKFKDRVTLTVDTSISTA | HSGVPSRFSGSGSGTEFTLTISSLQPEDF |
| | YMELSRLRSDDTAVYYCARLGDNYRG | ATYYCQQYSTYLYTFGGGTKLEIKRTV |
| | YFDVWGQGTTVTVSSASTKGPSVFPLA | AAPSVFIFPPSDEQLKSGTASVVCLLNN |
| | PSSKSTSGGTAALGCLVKDYFPEPVTVS | FYPREAKVQWKVDNALQSGNSQESVT |
| | WNSGALTSGVHTFPAVLQSSGLYSLSS | EQDSKDSTYSLSSTLTLSKADYEKHKV |
| | VVTVPSSSLGTQTYICNVNHKPSNTKV | YACEVTHQGLSSPVTKSFNRGEC (SEQ |
| | DKKVEPKSCDKTHTCPPCPAPELLGGP | ID NO: 116) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.8 | QVQLVQSGAEVKKPGASVKVSCKASG | DIQLTQSPSFLSASVGDRVTITCRASQD |
| | YSFTGYTMNWVRQAPGQNLEWIGLIN | VGSAVVWYQQKPGKAPKLLIDAASIRH |
| | PATGGTYYNQKFKDRVTLTVDTSISTA | SGVPSRFSGSGSGTEFTLTISSLQPEDFA |
| | YMELSRLRSDDTAVYYCARLGDNYRG | TYYCQQYSTYLYTFGGGTKLEIKRTVA |
| | YFDVWGQGTTVTVSSASTKGPSVFPLA | APSVFIFPPSDEQLKSGTASVVCLLNNF |
| | PSSKSTSGGTAALGCLVKDYFPEPVTVS | YPREAKVQWKVDNALQSGNSQESVTE |
| | WNSGALTSGVHTFPAVLQSSGLYSLSS | QDSKDSTYSLSSTLTLSKADYEKHKVY |
| | VVTVPSSSLGTQTYICNVNHKPSNTKV | ACEVTHQGLSSPVTKSFNRGEC (SEQ |
| | DKKVEPKSCDKTHTCPPCPAPELLGGP | ID NO: 117) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 111) | |
| 10A3D6hum8.9 | QVQLVQSGAEVKKPGASVKVSCKASG | DIQLTQSPSFLSASVGDRVTITCRASQD |
| | YSFTGYTMNWVRQAPGQGLEWIGLIN | VGSAVVWYQQKPGKAPKLLIDAASIRH |
| | PATGGTYYNQKFKDRVTLTVDTSISTA | SGVPSRFSGSGSGTEFTLTISSLQPEDFA |
| | YMELSRLRSDDTAVYYCARLGDNYRG | TYYCQQYSTYLYTFGGGTKLEIKRTVA |
| | YFDVWGQGTTVTVSSASTKGPSVFPLA | APSVFIFPPSDEQLKSGTASVVCLLNNF |
| | PSSKSTSGGTAALGCLVKDYFPEPVTVS | YPREAKVQWKVDNALQSGNSQESVTE |
| | WNSGALTSGVHTFPAVLQSSGLYSLSS | QDSKDSTYSLSSTLTLSKADYEKHKVY |
| | VVTVPSSSLGTQTYICNVNHKPSNTKV | ACEVTHQGLSSPVTKSFNRGEC (SEQ |
| | DKKVEPKSCDKTHTCPPCPAPELLGGP | ID NO: 117) |
| | SVFLFPPKPKDTLMISRTPEVTCVVVDV | |
| | SHEDPEVKFNWYVDGVEVHNAKTKPR | |
| | EEQYNSTYRVVSVLTVLHQDWLNGKE | |
| | YKCKVSNKALPAPIEKTISKAKGQPREP | |
| | QVYTLPPSRDELTKNQVSLTCLVKGFY | |
| | PSDIAVEWESNGQPENNYKTTPPVLDS | |
| | DGSFFLYSKLTVDKSRWQQGNVFSCSV | |
| | MHEALHNHYTQKSLSLSPGK (SEQ ID | |
| | NO: 77) | |

TABLE 58
Sequences of Exemplary Constant Regions

| | | |
|---|---|---|
| Human | G1m(z) | ASTKGPSVFPLAPSSKSTSGGTAALGCLVKDYFPEPVTVSWN |
| IgG1 | allele | SGALTSGVHTFPAVLQSSGLYSLSSVVTVPSSSLGTQTYICN |
| Constant | (K214/D356/L358) | VNHKPSNTKVDKKVEPKSCDKTHTCPPCPAPELLGGPSVFLF |
| (IGHG1) | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LPAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK (SEQ ID NO:118) |
| Human | G1m(z) | ASTKGPSVFPLAPSSKSTSGGTAALGCLVKDYFPEPVTVSWN |
| IgG1 | allele | SGALTSGVHTFPAVLQSSGLYSLSSVVTVPSSSLGTQTYICNVN |
| Constant | with | HKPSNTKVDKKVEPKSCDKTHTCPPCPAPEAAGAPSVFLFPPK |
| (IGHG1) | L234A/ | PKDTLMISRTPEVTCVVVDVSHEDPEVKFNWYVDGVEVHNAKT |
| LALAGA | L235A/G237A | KPREEQYNSTYRVVSVLTVLHQDWLNGKEYKCKVSNKALPAPI |
| | | EKTISKAKGQPREPQVYTLPPSRDELTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK (SEQ ID NO: 119) |
| Human | WT | LGGPSVFLFPPKPKDTLMISRTPEVTCVVVDVS |
| IgG1 Fc | | HEDPEVKFNWYVDGVEVHNAKTKPREEQYNSTYRVVSVLTVLHQDWLNGK |
| region | | EYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO: 120) |
| Human | Uniprot | RTVAAPSVFIFPPSDEQLKSGTASVVCLLNNFYPREAKVQWK |
| IgK | P01834 | VDNALQSGNSQESVTEQDSKDSTYSLSSTLTLSKADYEKHKVY |
| Constant | | ACEVTHQGLSSPVTKSFNRGEC (SEQ ID NO: 121) |
| (IGKC) | | |
| Mouse | Uniprot | AKTTPPSVYPLAPGSAAQTNSMVTLGCLVKGYFPEPVTVTWN |
| IgG1 | P01868 | SGSLSSGVHTFPAVLQSDLYTLSSSVTVPSSPRPSETVTCNV |
| Constant | | AHPASSTKVDKKIVPRDCGCKPCICTVPEVSSVFIFPPKPKD |
| (IGHG1 | | VLTITLTPKVTCVVVDISKDDPEVQFSWFVDDVEVHTAQTQP |
| Mouse) | | REEQFNSTFRSVSELPIMHQDWLNGKEFKCRVNSAAFPAPIE |
| | | KTISKTKGRPKAPQVYTIPPPKEQMAKDKVSLTCMITDFFPE |
| | | DITVEWQWNGQPAENYKNTQPIMNTNGSYFVYSKLNVQKSNW |
| | | EAGNTFTCSVLHEGLHNHHTEKSLSHSPGK (SEQ ID NO: 122) |
| Mouse | Uniprot | KTTPPSVYPLAPGCGDTTGSSVTLGCLVKGYFPESVTVTWNS |
| IgG2b | P01867 | GSLSSSVHTFPALLQSGLYTMSSSVTVPSSTWPSQTVTCSVA |
| Constant | | HPASSTTVDKKLEPSGPISTINPCPPCKECHKCPAPNLEGGP |
| (IGG2B | | SVFIFPPNIKDVLMISLTPKVTCVVVDVSEDDPDVQISWFVN |
| Mouse) | | NVEVHTAQTQTHREDYNSTIRVVSTLPIQHQDWMSGKEFKCK |
| | | VNNKDLPSPIERTISKIKGLVRAPQVYILPPPAEQLSRKDVS |
| | | LTCLVVGFNPGDISVEWTSNGHTEENYKDTAPVLDSDGSYFI |
| | | YSKLNMKTSKWEKTDSFSCNVRHEGLKNYYLKKTISRSPGLD |
| | | LDDICAEAKDGELDGLWTTITIFISLFLLSVCYSASVTLFKV |
| | | KWIFSSVVELKQKISPDYRNMIGQGA (SEQ ID NO: 123) |
| Mouse | Uniprot | RADAAPTVSIFPPSSEQLTSGGASVVCFLNNFYPKDINVKWK |
| IgK | P01837 | IDGSERQNGVLNSWTDQDSKDSTYSMSSTLTLTKDEYERHNSYT |
| Constant | | CEATHKTSTSPIVKSFNRNEC (SEQ ID NO: 124) |
| (IGKC | | |
| Mouse) | | |

### Example 9. Depletion of KLRB1+ T Cells in Cynomolgus Monkey by 10A3D6Hum2.2-Afuc (Afucosylated 10A3D6Hum2.2) at 3 mg/kg and 10 mg/kg

Antibody 10A3D6hum2.2-afuc was administered intravenously to two cynomolgus monkeys in a single dose of 3 mg/kg in one monkey and 10 mg/kg in another. Blood was analyzed by FACS to detect changes in immune cell populations, using the anti-KLRB1 (anti-CD161) antibody 57E2E4 to detect KLRB1+ cells. These studies showed near complete depletion of the KLRB1+CD8+ T cell population at Day 1, 3, and 7 (FIG. 31).

### Example 10. Depletion of KLRB1+ T Cells in Cynomolgus Monkey by 10A3D6Hum2.2-Afuc (Afucosylated 10A3D6Hum2.2) at 0.1 mg/kg, 0.3 mg/kg, 1 mg/kg, and 3 mg/kg

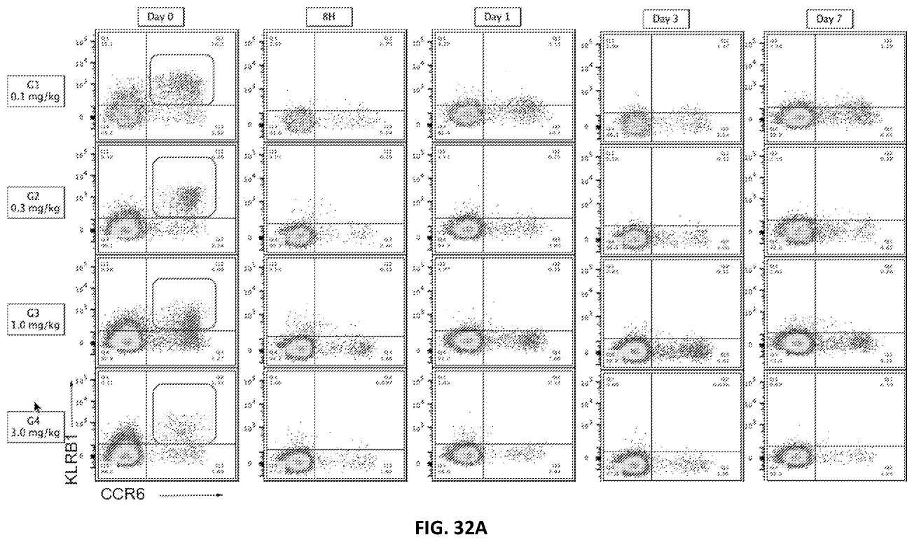

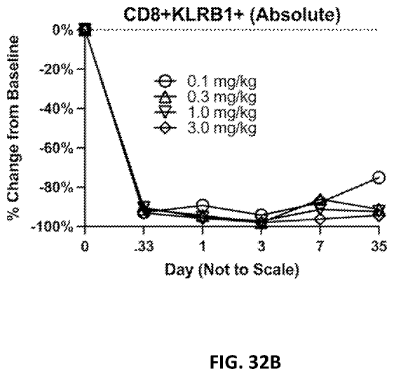

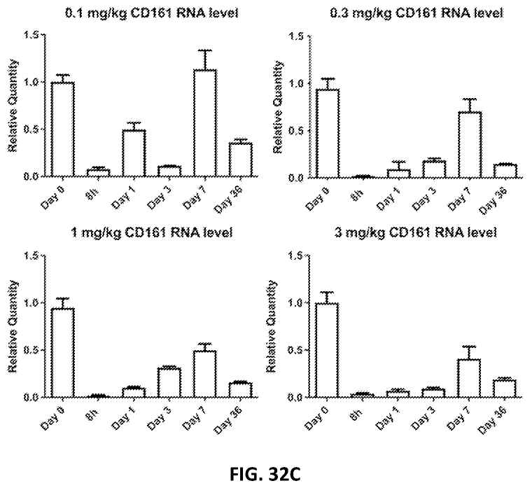

Antibody 10A3D6hum2.2-afuc was administered intravenously to four cynomolgus monkeys in a single dose of 0.1 mg/kg in one monkey, 0.3 mg/kg in one monkey, 1 mg/kg in one monkey, and 3 mg/kg in one monkey. Blood was analyzed by FACS to detect changes in immune cell populations, using the anti-KLRB1 antibody 57E2E4 to detect KLRB1+ cells. These studies showed near complete depletion of the KLRB1+CD8+ T cell population at 8 hours post-dose, Day 1, 3, 7, and 35 (FIG. 32A and FIG. 32B). Gene expression of KLRB1 transcript by quantitative PCR (qPCR) similarly showed depletion of KLRB1 transcript (FIG. 32C).

### Example 11. Pharmacokinetic Parameters of 10A3D6Hum2.2-Afuc in Cynomolgus Monkey

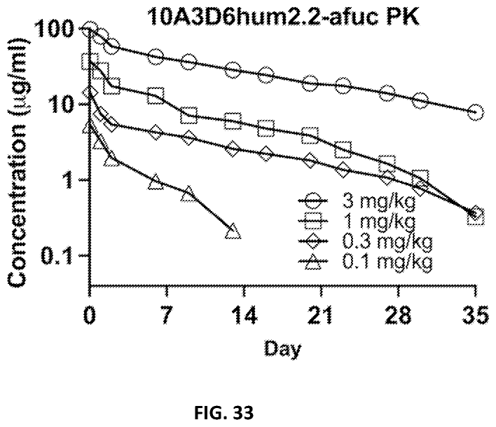

Antibody 10A3D6hum2.2-afuc was administered intravenously to four cynomolgus monkeys in a single dose of 0.1 mg/kg in one monkey, 0.3 mg/kg in one monkey, 1 mg/kg in one monkey, and 3 mg/kg in one monkey. Serum concentration of 10A3D6hum2.2-afuc was measured and demonstrated acceptable pharmacokinetic parameters (FIG. 33 and Table 59).

TABLE 59
Pharmacokinetic parameters

| PK Parameter | Beta T_{1/2} | CL | MRTₗₐₛₜ | AUCₗₐₛₜ | Vss |
|---|---|---|---|---|---|
| Unit | day | mL/day/kg | day | day*ug/mL | mL/kg |
| 0.1 mg/kg IV | 3.51 | 4.8 | 4.67 | 20.8 | 27.3 |
| 0.3 mg/kg IV | 5.08 | 2.97 | 11.42 | 100.9 | 33.9 |
| 1.0 mg/kg IV | 3.37 | 3.92 | 8.71 | 255.2 | 34.2 |
| 3.0 mg/kg IV | 9.63 | 2.61 | 15.21 | 1152.2 | 39.6 |

### Example 12. Suppression of Th17 Pathway by 10A3D6Hum2.2-Afuc (Afucosylated 10A3D6Hum2.2) in Cynomolgus Monkey

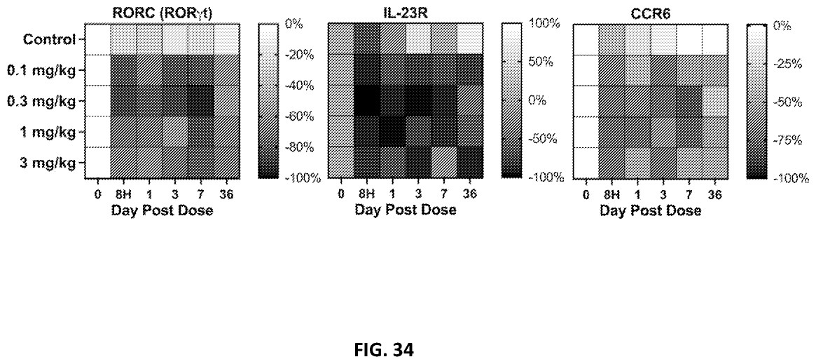

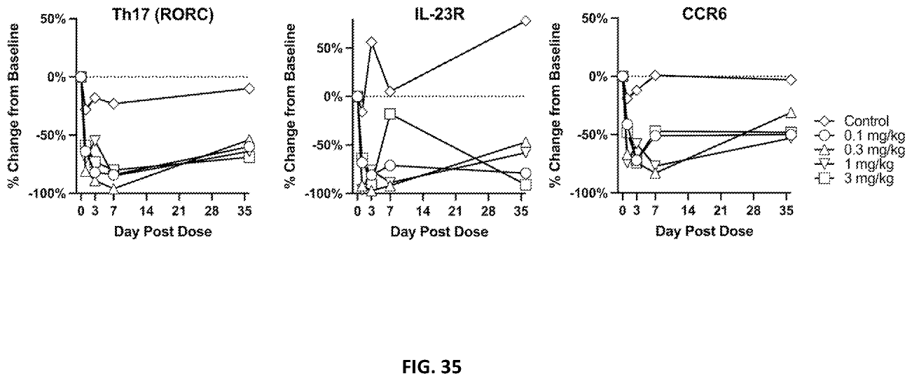

Antibody 10A3D6hum2.2-afuc was administered intravenously to four cynomolgus monkeys in a single dose of 0.1 mg/kg in one monkey, 0.3 mg/kg in one monkey, 1 mg/kg in one monkey, and 3 mg/kg in one monkey. Blood was analyzed by RNAseq to detect changes in Th17 pathway transcripts These studies showed marked suppression of the key Th17 transcription factor RORC and key Th17 cell markers IL-23R and CCR6 receptors (FIGS. 34 and 35).

### Example 13. Suppression of peTh2 Pathway by 10A3D6Hum2.2-Afuc (Afucosylated 10A3D6Hum2.2) in Cynomolgus Monkey

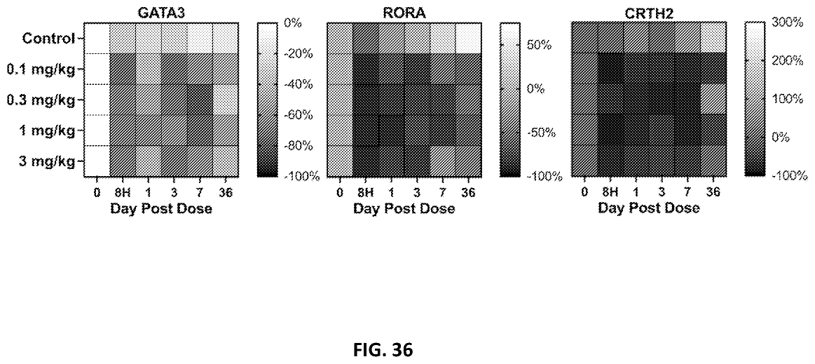

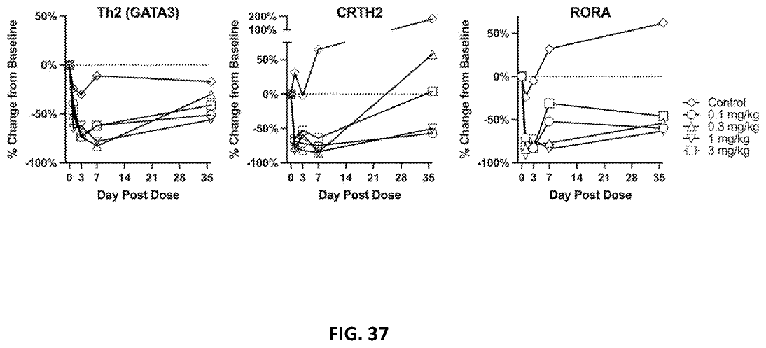

Antibody 10A3D6hum2.2-afuc was administered intravenously to four cynomolgus monkeys in a single dose of 0.1 mg/kg in one monkey, 0.3 mg/kg in one monkey, 1 mg/kg in one monkey, and 3 mg/kg in one monkey. Blood was analyzed by RNAseq to detect changes in pathogenic effector Th2 (peTh2) pathway transcripts. These studies showed marked suppression of the key peTh2 transcription factors GATA3 and RORA, and key peTh2 cell marker CRTH2 (FIGS. 36 and 37).

### Example 14. Suppression of Th1 Pathway by 10A3D6Hum2.2-Afuc (Afucosylated 10A3D6Hum2.2) in Cynomolgus Monkey

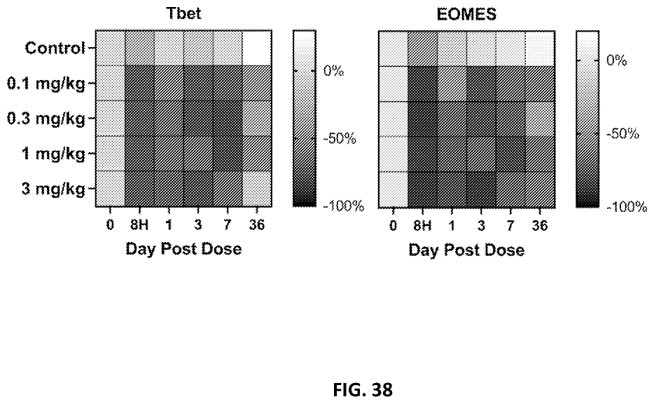

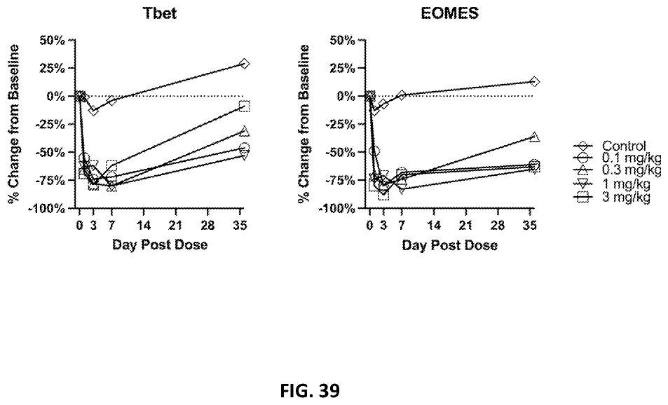

Antibody 10A3D6hum2.2-afuc was administered intravenously to four cynomolgus monkeys in a single dose of 0.1 mg/kg in one monkey, 0.3 mg/kg in one monkey, 1 mg/kg in one monkey, and 3 mg/kg in one monkey. Blood was analyzed by RNAseq to detect changes in Th1 and CD8 effector pathway transcripts These studies showed marked suppression of the key Th1 and CD8 effector transcription factors Tbet (TBX21) and EOMES (FIGS. 38 and 39).

### Example 15. KLRB1 ECD Binding

Antibodies disclosed herein and appropriate controls are assayed for binding against human and cynomolgus KLRB1 protein extracellular domain (ECD) via ELISA. ELISA plates are coated with KLRB1 ECD at 1 μg/ml, at 4° C., overnight prior to adding primary antibody (for human, 3 nM starting concentration, and 3-fold dilutions thereof; for cynomolgus, 10 nM starting concentration and 5-fold dilutions thereof) at 37° C. for 1 hour and then a labeled secondary anti-human IgG antibody 1:5000 at 37° C. for 45 minutes. The amount of antibody bound to KLRB1 is determined by the intensity of the signal of the bound second antibody.

Antibodies disclosed herein and appropriate controls (e.g., antibodies known in the art or described herein, e.g., B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7)) are assayed for binding against human or cynomolgus KLRB1 extracellular domain (ECD) via surface plasma resonance (SPR) or similar methods, such as biolayer interferometry (BLI). SPR is performed using Biacore, in which the binding of a ligand to its receptor is detected by changes in a refractive index on a sensor's surface. BLI is performed using Octet, in which the binding of a ligand to its receptor is detected by changes in optical thickness in biolayers.

The disclosed antibodies may have increased binding potency and lower binding EC₅₀ to cell expressed KLRB1 or to soluble KLRB1 extracellular domain compared to the controls.

### Example 16. Antibody-Dependent Cell-Mediated Cytotoxicity (ADCC) Activation

Antibodies disclosed herein and appropriate controls (e.g., antibodies known in the art or described herein, e.g., B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7)) are assayed for antibody-dependent cell-mediated cytotoxicity (ADCC) using a reporter assay (Genscript ADCC assay with a reporter cell line GS-J2 that expresses CD16A). CHO-K1 cells expressing human KLRB1 (CHO-hum-KLRB1) are incubated with luminescent reporter cells GS-J2/CD16A and various antibodies. In a separate assay, ADCC elicited by antibodies are evaluated using a cell lysis assay involving LDH release and detection of formazan salt (Genscript, SC1544). CHO-hum-KLRB1 are incubated with human peripheral blood mononuclear cells (PBMCs) and antibodies. The antibodies demonstrate increased ADCC mediated depletion potency and lower EC₅₀ for depletion of CHO-KLRB1+ cells or lower EC₅₀ for reporter cell line ADCC assay compared to the controls.

### Example 17. Additional Biochemical and Biophysical Characterization of the Antibodies

Competition assays of antibodies disclosed herein and appropriate controls (e.g., antibodies known in the art or described herein, e.g., B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), or an antibody described in US20210122826 (i.e., KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7)) with biotin labeled CLEC2D are performed by FACS. CHO-hum-KLRB1 or CHO-cyno-KLRB1 cells are incubated with biotin labeled CLEC2D and the fluorescent signal detected by FACS. The ability of antibodies to block CLEC2D across a concentration range (e.g., 0 to 200 nM) and result in loss or gain of fluorescent signal is used to measure the IC₅₀ potency of inhibition, or of augmentation, of the antibodies.

The antibodies have increased binding affinity kinetics (lower K_{D} or lower K_{off}), lower IC₅₀ for inhibition of CLEC2D binding, e.g., binding to cell expressing KLRB1, and therefore have increased blocking potency; and lower EC₅₀ for augmenting (increasing) CLEC2D binding, e.g., binding to cell expressed KLRB1, and therefore have increased augmentation potency, when compared to the controls.

Differential scanning calorimetry (DSC) is conducted to measure antibody Tonset (onset temperature at which the melting/unfolding is first detected) and Tm (melting temperature). The antibodies have improved biophysical parameters, such as higher melting temperature, greater freeze-thaw stability, lower isomerization, reduced or absence of deamidation, and/or less susceptibility to oxidation, when compared to the controls.

### REFERENCES

Almagro, J. C., et al. (2008). “Humanization of antibodies.” Front Biosci 13:1619-1633.
Aldemir, H., et al. (2005). “Cutting edge: lectin-like transcript 1 is a ligand for the CD161 receptor.” J Immunol 175 (12): 7791-7795.
Basdeo, S. A., et al. (2017). “Ex-Th17 (Nonclassical Th1) Cells Are Functionally Distinct from Classical Th1 and Th17 Cells and Are Not Constrained by Regulatory T Cells.” J Immunol 198 (6): 2249-2259.
Fergusson, J. R., et al. (2014). “CD161 defines a transcriptional and functional phenotype across distinct human T cell lineages.” Cell Rep 9 (3): 1075-1088.
Maggi, L., et al. (2010). “CD161 is a marker of all human IL-17-producing T-cell subsets and is induced by RORC.” Eur J Immunol 40 (8): 2174-81.
Mathewson, N. D., et al. (2021). “Inhibitory CD161 receptor identified in glioma-infiltrating T cells by single-cell analysis.” Cell 184 (5): 1281-1298.
Satoh, M., et al. (2008). “Non-fucosylated therapeutic antibodies as next-generation therapeutic antibodies.” Expert Opin Biol Ther 6 (11): 1161-1173.
Yang, J., et al. (2014). “Targeting Th17 cells in autoimmune diseases.” Trends Pharmacol Sci 35 (10): 493-500.

What is claimed is:

1. An antibody or antigen binding portion thereof that specifically binds to killer cell lectin-like receptor subfamily B, member 1, (KLRB1; optionally SEQ ID NO: 1 or SEQ ID NO: 2), wherein the antibody or antigen binding portion thereof comprises at least one of
(a) a heavy chain variable region (VH) comprising a VH complementarity determining region (CDR) 1 comprising a sequence that is at least 95% identical to a VH CDR1 amino acid sequence set forth in one of Table 1 to 53, preferably Table 1, 30, 31, 32, or 45 to 53; a VH CDR2 comprising a sequence that is at least 95% identical to a VH CDR2 amino acid sequence set forth in one of Table 1 to 53, preferably Table 1, 30, 31, or 32, and a VH CDR3 comprising a sequence that is at least 95% identical to a VH CDR3 amino acid sequence set forth in one of Table 1 to 53, preferably Table 1, 30, 31, 32, or 45 to 53; and
(b) a light chain variable region (VL) comprising a VL CDR1 comprising a sequence that is at least 95% identical to a VL CDR1 amino acid sequence set forth in one of Table 1 to 53, preferably Table 1, 30, 31, 32, or 45 to 53, a VL CDR2 comprising a sequence that is at least 95% identical to a VL CDR2 amino acid sequence set forth in one of Table 1 to 53, preferably Table 1, 30, 31, 32, or 45 to 53, and a VL CDR3 comprising a sequence that is at least 95% identical to a VL CDR3 amino acid sequence set forth in one of Table 1 to 53, preferably Table 1, 30, 31, 32, or 45 to 53.

2. The antibody or antigen binding portion thereof of claim 1, wherein the VH comprises or consists of an VH amino acid sequence set forth in Table 1, 30, 31, 32, or 45 to 53.

3. The antibody or antigen binding portion thereof of claim 1, wherein the VL comprises or consists of an VL amino acid sequence set forth in Table 1, 30, 31, 32, or 45 to 53.

4. The antibody or antigen binding portion thereof of claim 1, wherein the VH comprises or consists of the VH amino acid sequence set forth in Table 1, and the VL comprises or consists of the VH amino acid sequence set forth in Table 1.

5. The antibody or antigen binding portion thereof of claim 1, wherein the VH comprises or consists of the VH amino acid sequence set forth in Table 30, and the VL comprises or consists of the VH amino acid sequence set forth in Table 30.

6. The antibody or antigen binding portion thereof of claim 1, wherein the VH comprises or consists of the VH amino acid sequence set forth in Table 31, and the VL comprises or consists of the VH amino acid sequence set forth in Table 31.

7. The antibody or antigen binding portion thereof of claim 1, wherein the VH comprises or consists of the VH amino acid sequence set forth in Table 32, and the VL comprises or consists of the VH amino acid sequence set forth in Table 32.

8. The antibody or antigen binding portion thereof of claim 1, wherein the VH comprises or consists of the VH amino acid sequence set forth in Table 45, and the VL comprises or consists of the VH amino acid sequence set forth in Table 45.

9. The antibody or antigen binding portion thereof of claim 1, wherein the VH comprises or consists of the VH amino acid sequence set forth in Table 46, and the VL comprises or consists of the VH amino acid sequence set forth in Table 46.

10. The antibody or antigen binding portion thereof of claim 1, wherein the VH comprises or consists of the VH amino acid sequence set forth in Table 47, and the VL comprises or consists of the VH amino acid sequence set forth in Table 47.

11. The antibody or antigen binding portion thereof of claim 1, wherein the VH comprises or consists of the VH amino acid sequence set forth in Table 48, and the VL comprises or consists of the VH amino acid sequence set forth in Table 48.

12. The antibody or antigen binding portion thereof of claim 1, wherein the VH comprises or consists of the VH amino acid sequence set forth in Table 49, and the VL comprises or consists of the VH amino acid sequence set forth in Table 49.

13. The antibody or antigen binding portion thereof of claim 1, wherein the VH comprises or consists of the VH amino acid sequence set forth in Table 50, and the VL comprises or consists of the VH amino acid sequence set forth in Table 50.

14. The antibody or antigen binding portion thereof of claim 1, wherein the VH comprises or consists of the VH amino acid sequence set forth in Table 51, and the VL comprises or consists of the VH amino acid sequence set forth in Table 51.

15. The antibody or antigen binding portion thereof of claim 1, wherein the VH comprises or consists of the VH amino acid sequence set forth in Table 52, and the VL comprises or consists of the VH amino acid sequence set forth in Table 52.

16. The antibody or antigen binding portion thereof of claim 1, wherein the VH comprises or consists of the VH amino acid sequence set forth in Table 53, and the VL comprises or consists of the VH amino acid sequence set forth in Table 53.

17. The antibody or antigen binding portion thereof of claim 1, wherein the VH comprises or consists of an amino acid sequence having at least 95% sequence identity to the VH amino acid sequence set forth in Table 1, and the VL comprises or consists of an amino acid sequence having at least 95% sequence identity to the VL amino acid sequence set forth in Table 1.

18. The antibody or antigen binding portion thereof of claim 1, wherein the VH comprises or consists of an amino acid sequence having at least 95% sequence identity to the VH amino acid sequence set forth in one of Tables 30, 31, 32, or 45 to 53, and the VL comprises or consists of an amino acid sequence having at least 95% sequence identity to the amino acid sequence set forth in one of Tables 30, 31, 32, or 45 to 53, preferably wherein the VH and VL are from the same Table.

19. The antibody or antigen binding portion thereof of claims 1-18, which comprises a constant region heavy chain and light chain, wherein the constant region heavy chain and/or light chain comprises or consists of an amino acid sequence set forth in Table 58.

20. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 1 and a variable region light chain consisting of the LC amino acid sequence set forth in Table 1, and optionally a constant region, optionally wherein the antibody or antigen binding portion thereof comprises a sequence that is at least 95% identical to a sequence set forth in Table 57.

21. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 30 and a variable region light chain consisting of the LC amino acid sequence set forth in Table 30, and optionally a constant region, optionally wherein the antibody or antigen binding portion thereof comprises a sequence that is at least 95% identical to a sequence set forth in Table 57.

22. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 31, and a variable region light chain consisting of the LC amino acid sequence set forth in Table 31, and optionally a constant region, optionally wherein the antibody or antigen binding portion thereof comprises a sequence that is at least 95% identical to a sequence set forth in Table 57.

23. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 32, and a variable region light chain consisting of the LC amino acid sequence set forth in Table 32, and optionally a constant region,

24. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 45, and a variable region light chain consisting of the LC amino acid sequence set forth in Table 45, and optionally a constant region, optionally wherein the antibody or antigen binding portion thereof comprises a sequence that is at least 95% identical to a sequence set forth in Table 57.

25. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 46, and a variable region light chain consisting of the LC amino acid sequence set forth in Table 46, and optionally a constant region, optionally wherein the antibody or antigen binding portion thereof comprises a sequence that is at least 95% identical to a sequence set forth in Table 57.

26. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 47, and a variable region light chain consisting of the LC amino acid sequence set forth in Table 47, and optionally a constant region, optionally wherein the antibody or antigen binding portion thereof comprises a sequence that is at least 95% identical to a sequence set forth in Table 57.

27. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 48, and a variable region light chain consisting of the LC amino acid sequence set forth in Table 48, and optionally a constant region,

28. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 49, and a variable region light chain consisting of the LC amino acid sequence set forth in Table 49, and optionally a constant region, optionally wherein the antibody or antigen binding portion thereof comprises a sequence that is at least 95% identical to a sequence set forth in Table 57.

29. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 50, and a variable region light chain consisting of the LC amino acid sequence set forth in Table 50, and optionally a constant region, optionally wherein the antibody or antigen binding portion thereof comprises a sequence that is at least 95% identical to a sequence set forth in Table 57.

30. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 51, and a variable region light chain consisting of the LC amino acid sequence set forth in Table 51, and optionally a constant region, optionally wherein the antibody or antigen binding portion thereof comprises a sequence that is at least 95% identical to a sequence set forth in Table 57.

31. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 52, and a variable region light chain consisting of the LC amino acid sequence set forth in Table 52, and optionally a constant region,

32. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 53, and a variable region light chain consisting of the LC amino acid sequence set forth in Table 53, and optionally a constant region, optionally wherein the antibody or antigen binding portion thereof comprises a sequence that is at least 95% identical to a sequence set forth in Table 57.

33. The antibody or antigen binding portion thereof of claim 1, further comprising a heavy chain constant region hinge region and Fc domain.

34. The antibody or antigen binding portion thereof of claim 1, which is an antibody that comprises a heavy chain constant region comprising an amino acid sequence having at least 80%, 90%, 95%, or 97% sequence identity to a heavy chain constant region amino acid sequence set forth in Table 58.

35. The antibody or antigen binding portion thereof of claim 1, wherein the antibody is a monoclonal antibody.

36. The antibody or antigen binding portion thereof of claim 1, wherein the antibody is a chimeric, humanized, or human antibody, and/or comprises one or more mutations (optionally in a CDR) that remove Asn (N)-glycosylation sites or remove Cys, Asp, Met, Trp or Lys.

37. The antibody or antigen binding portion thereof of claim 1, wherein the antibody is an immunoglobulin G (IgG) subtype IgG1 antibody, an IgG2 antibody, or an IgG4 antibody.

38. The antibody or antigen binding portion thereof of claim 1, which is an antibody that comprises an Fc region, preferably human IgG1, that binds to Fc gamma receptors (FcγRs) and induces antibody dependent cell-mediated cytotoxicity (ADCC) to deplete cells expressing KLRB1, or that binds to C1q and induce complement dependent cytotoxicity (CDC).

39. The antibody or antigen binding portion thereof of claim 1, which is conjugated to a cytotoxic agent.

40. The antibody or antigen binding portion thereof of claim 1, which comprises an Fc region that is afucosylated

41. A polynucleotide comprising a nucleic acid sequence encoding an antibody or antigen binding portion thereof of claims 1 to 40.

42. The polynucleotide of claim 41, wherein the nucleic acid sequence is operably linked to a promoter.

43. A vector comprising the polynucleotide of any one of claims 41-42.

44. A host cell comprising the polynucleotide of any one of claims 41-42, or the vector of claim 43, and optionally expressing the antibody or antigen binding portion thereof.

45. A pharmaceutical composition that comprises the antibody or antigen binding portion thereof of any one of the preceding claims, and a pharmaceutically acceptable carrier or diluent.

46. A method of making the antibody or antigen binding portion thereof of any one of the preceding claims, comprising culturing the host cell of claim 44 under conditions sufficient to express the antibody or antigen binding portion thereof and isolating the antibody or antigen binding portion thereof.

47. The method of claim 46, further comprising formulating the antibody as a pharmaceutical composition.

48. A method of treating one or more of an autoimmune disease, an allergic disease, a transplant rejection, and a hematologic malignancy in a subject in need thereof, the method comprising administering to the subject an antibody that binds KLRB1, preferably the antibody of any one of claims 1-41, the polynucleotide of any one of claims 41-42, the vector of claim 43, the pharmaceutical composition of claim 45, or the host cell of claim 44 that expresses the antibody or antigen binding portion thereof.

49. The method of claim 48, wherein the autoimmune disease is autoimmune hepatitis, rheumatoid arthritis, Sjogren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, or hidradenitis suppurativa.

50. The method of claim 48, wherein the allergic disease is asthma, allergic eosinophilic asthma, allergy, atopic dermatitis, nasal polyposis, eosinophilic gastrointestinal disorder, or hypereosinophilic syndrome.

51. The method of claim 48, wherein the transplant rejection can be a rejection of a kidney, lung, heart, liver, limb, skin, or multi-organ transplant.

52. The method of claim 48, wherein the hematological malignancy is a lymphoma or leukemia.

53. The method of claim 52, wherein the lymphoma is NK/T cell lymphoma, mycosis fungoides, Sezary syndrome, peripheral T cell lymphoma, angioimmunoblastic T cell lymphoma (AITL), or peripheral T cell lymphoma not otherwise specified (PTCL-NOS).

54. The method of claim 52, wherein the leukemia is T cell leukemia, aggressive NK cell leukemia, T cell prolymphocytic leukemia (T-PLL), or large granular lymphocytic leukemia (LGLL).

55. The antibody of any one of claims 1-40, the polynucleotide of any one of claims 41-42, the vector of claim 43, or the method of claims 48-54, wherein the antibody binds to and depletes Th17, Th17.1, ex-Th17, Tc17, mucosal-associated invariant T cells (MAIT), invariant NK-T cells (iNKT), innate lymphoid cells types 2 and 3 (ILC2, and ILC3), pathogenic effector Th2 (peTh2) cells, NK cells, and/or neoplastic T or NK cells expressing KLRB1.

56. The antibody of any one of claims 1-40, which is not clone B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), or JNH25G2G22 (Creative Diagnostics). B199.2 (Invitrogen), 191B8 (Miltenyi), DX12 (BD Biosciences), JNH25G2G22 (Creative Diagnostics), KW1, KW1.2.1, KW1.3.12, KW7, KW7.2.2, KW7.3.7, KW9, KW9.3.3, KW17, KW17.3.4, KM12, KM12.2.3, KM12.3.2, or KM12.4.7.

57. The antibody of any one of claims 1-40, the polynucleotide of any one of claims 41-42, or the vector of claim 43, for use in a method of treating one or more of an autoimmune disease, an allergic disease, a transplant rejection, and a hematologic malignancy in a subject in need thereof.

58. The antibody, polynucleotide, or vector for the use of claim 57, wherein the autoimmune disease is autoimmune hepatitis, rheumatoid arthritis, Sjogren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, or hidradenitis suppurativa.

59. The antibody, polynucleotide, or vector for the use of claim 57, wherein the allergic disease is asthma, allergic eosinophilic asthma, allergy, atopic dermatitis, nasal polyposis, eosinophilic gastrointestinal disorder, or hypereosinophilic syndrome.

60. The antibody, polynucleotide, or vector for the use of claim 57, wherein the transplant rejection can be a rejection of a kidney, lung, heart, liver, limb, skin, or multi-organ transplant.

61. The antibody, polynucleotide, or vector for the use of claim 57, wherein the hematological malignancy is a lymphoma or leukemia.

62. The antibody, polynucleotide, or vector for the use of claim 61, wherein the lymphoma is hepatosplenic T cell lymphoma, NK/T cell lymphoma, mycosis fungoides, Sezary syndrome, peripheral T cell lymphoma, angioimmunoblastic T cell lymphoma (AITL), or peripheral T cell lymphoma not otherwise specified (PTCL-NOS).

63. The antibody, polynucleotide, or vector for the use of claim 61, wherein the leukemia is T cell leukemia, NK cell leukemia, T cell prolymphocytic leukemia (T-PLL), or large granular lymphocytic leukemia (LGLL).
